(19) United States
(12) Patent Application Publication
(10) Pub. No.: US 2018/0193333 A1
(43) Pub. Date: Jul. 12, 2018

(54) TRANSDERMAL DELIVERY SYSTEM

(71) Applicant: PURDUE PHARMA L.P., Stamford, CT (US)

(72) Inventors: Gabriel Wauer, Bad Neuenahr-Ahrweiler (DE); Thomas Hille, Neuwied (DE); Kevin John Smith, Cambridge (GB); Gillian Elizabeth Mundin, Cambridge (GB); Helen Elizabeth Johnson, Cambridge (GB)

(73) Assignee: PURDUE PHARMA L.P., Stamford, CT (US)

(21) Appl. No.: 15/915,369

(22) Filed: Mar. 8, 2018

Related U.S. Application Data
(63) Continuation of application No. 14/650,451, filed on Jun. 8, 2015.
(60) Provisional application No. 61/736,342, filed on Dec. 12, 2012.

(51) Int. Cl. A61K 31/485; A61K 47/32; A61K 47/12; A61K 9/70

(52) U.S. Cl. CPC A61K 31/485; A61K 47/32; A61K 47/12; A61K 9/7092; A61K 9/7069; A61K 9/7084

(57) ABSTRACT

The invention relates to a method of treating pain in a patient by applying a transdermal therapeutic system for the transdermal administration of buprenorphine for 7 days on the skin of a patient, said transdermal therapeutic system comprising a buprenorphine-containing self-adhesive layer structure comprising A) a buprenorphine-impermeable backing layer, and B) a buprenorphine-containing matrix layer on said buprenorphine-impermeable backing layer, the matrix layer comprising a) a polymer base, b) buprenorphine, and c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and C) a skin contact layer on said buprenorphine-containing matrix layer comprising a polymer-based pressure-sensitive adhesive, and optionally wherein the buprenorphine-containing self-adhesive layer structure contains said buprenorphine in an amount of less than 0.8 mg/cm2 buprenorphine base or an equimolar amount of a pharmaceutically acceptable salt thereof.

### TECHNICAL FIELD OF THE INVENTION

The present invention relates to a transdermal therapeutic system (TTS) for the transdermal administration of buprenorphine, a method of treating pain using said TTS, and a process of manufacturing said TTS.

### BACKGROUND OF THE INVENTION

The active ingredient buprenorphine (5R,6R,7R,9R,13S,145)-17-Cyclopropylmethyl-7-[(S)-3,3-dimethyl-2-hydroxybutan-2-yl]-6-methoxy-4,5-epoxy-6,14-ethanomorphinan-3-ol) is a partially synthetic opiate with high potency. Cancer patients may be treated with daily doses of around 1 mg. Despite its rather high molecular weight of 467.64 daltons, it is currently used for transdermal administration. The commercial TTS product Norspan®, also known as BuTrans® delivers buprenorphine to the skin sufficiently to treat patients in pain for a time period of 7 days (about 168 hours) and allows therefore a use of the TTS over a time period of 7 days and allows in a fixed dosing regimen a once-weekly TTS exchange. This is specifically beneficial in terms of convenience and patient compliance. Thus the overall efficacy of the pain medicament is enhanced. However, the long administration periods may cause problems with skin irritation, which in combination with the considerable size (i.e., area of release) of the TTS may be problematic. Also, the large amount of excess drug in the TTS necessary to sustain enough driving force for sustaining the appropriate drug delivery over the long period of time is costly and has the potential to be subject to illicit use.

It is therefore desirable to reduce the overall size (i.e., area of release) of the TTS as well as to reduce the total amount of buprenorphine in the TTS before administration and the amount remaining in the TTS after proper use (the residual amount). Thereby, the amount of drug available for illicit use (before and after proper use), and the amount to be wasted after proper use are both reduced. US Patent Application No. 2010/0119585 describes a certain TTS size and amount of drug reduction in comparison with the commercial TTS product Transtec® approved for an up-to-4 days administration regimen. Thus, the TTS needs to be replaced after 4 days at the latest. It is recommended to change Transtec® twice a week always on the same days at specific times, e.g. Monday mornings and Thursday evenings.

For convenience reasons it is, however, desirable to maintain the once weekly exchange mode (7 day dosing regimen) as, e.g., provided by the commercial product Norspan® instead of the every three to four days exchange mode as provided by, e.g., Transtec®.

All references and publications cited herein are hereby incorporated by reference in their enteritis for all purposes.

### OBJECTS AND SUMMARY OF THE INVENTION

It is an object of certain embodiments of the present invention to provide a method of treating pain in a patient by applying a transdermal therapeutic system for the transdermal administration of buprenorphine (e.g., buprenorphine base), which requires a relatively small amount of buprenorphine (e.g., buprenorphine base) contained therein.

It is an object of certain embodiments of the present invention to provide a method of treating pain in a patient by applying a transdermal therapeutic system for the transdermal administration of buprenorphine (e.g., buprenorphine base), which requires a relatively small area of release.

It is an object of certain embodiments of the present invention to provide a method of treating pain in a patient by applying a transdermal therapeutic system for the transdermal administration of buprenorphine (e.g., buprenorphine base), which requires a relatively small amount of buprenorphine (e.g., buprenorphine base) contained therein and optionally a relatively small area of release, and provides a release suitable for providing pain relief for 7 days (corresponding to about 168 hours or one week).

These objects and others are accomplished by the present invention, which according to one aspect relates to a method of treating pain in a patient by applying a transdermal therapeutic system for the transdermal administration of buprenorphine for 7 days on the skin of a patient, said transdermal therapeutic system comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
   and optionally wherein the buprenorphine-containing self-adhesive layer structure contains said buprenorphine in an amount of less than 0.8 mg/cm² buprenorphine base or an equimolar amount of a pharmaceutically acceptable salt thereof.

According to one aspect, the invention relates to a method of treating pain in a patient by applying a transdermal therapeutic system for the transdermal administration of buprenorphine base for 7 days on the skin of a patient, said transdermal therapeutic system comprising a buprenorphine base-containing self-adhesive layer structure comprising
A) a buprenorphine base-impermeable backing layer, and
B) a buprenorphine base-containing matrix layer on said buprenorphine base-impermeable backing layer, the matrix layer comprising
   a) a polymer-based pressure-sensitive adhesive comprising polysiloxane,
   b) buprenorphine base, and
   c) levulinic acid, in an amount sufficient so that said buprenorphine base is solubilized therein to form a mixture, and the levulinic acid buprenorphine base mixture forms dispersed deposits in the said pressure-sensitive adhesive, and
C) a skin contact layer on said buprenorphine base-containing matrix layer comprising a polymer-based pressure-sensitive adhesive comprising polyacrylate,
   and optionally wherein the buprenorphine base-containing self-adhesive layer structure contains said buprenorphine base in an amount of less than 0.8 mg/cm².

According to one aspect, the invention relates to a transdermal therapeutic system for the transdermal administration of buprenorphine comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
   and optionally wherein the buprenorphine-containing self-adhesive layer structure contains said buprenorphine in an amount of less than 0.8 mg/cm² buprenorphine base or an equimolar amount of a pharmaceutically acceptable salt thereof,
   in particular for use in a method of treating pain in a patient by applying the transdermal therapeutic system for 7 days on the skin of a patient.

According to one aspect, the invention relates to a transdermal therapeutic system for the transdermal administration of buprenorphine base comprising a buprenorphine base-containing self-adhesive layer structure comprising
A) a buprenorphine base-impermeable backing layer, and
B) a buprenorphine base-containing matrix layer on said buprenorphine base-impermeable backing layer, the matrix layer comprising
   a) a polymer-based pressure-sensitive adhesive comprising polysiloxane,
   b) buprenorphine base, and
   c) levulinic acid, in an amount sufficient so that said buprenorphine base is solubilized therein to form a mixture, and the levulinic acid buprenorphine base mixture forms dispersed deposits in the said pressure-sensitive adhesive, and
C) a skin contact layer on said buprenorphine base-containing matrix layer comprising a polymer-based pressure-sensitive adhesive comprising polyacrylate,
   and optionally wherein the buprenorphine base-containing self-adhesive layer structure contains said buprenorphine base in an amount of less than 0.8 mg/cm²,
   in particular for use in a method of treating pain in a patient by applying the transdermal therapeutic system for 7 days on the skin of a patient.

According to one aspect, the invention relates to a transdermal therapeutic system comprising buprenorphine for the transdermal administration of buprenorphine selected from a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a mean AUCt of more than 7,000 pg·hr/ml over about 168 hours of administration after a single-dose administration to a subject population; and a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a mean AUCt of more than 14,000 pg·hr/ml over about 168 hours of administration after a single-dose administration to a subject population; and a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a mean AUCt of more than 28,000 pg·hr/ml over about 168 hours of administration after a single-dose administration to a subject population; and a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a mean AUCt of more than 42,000 pg·hr/ml over about 168 hours of administration after a single-dose administration to a subject population; and a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a mean AUCt of more than 62,000 pg·hr/ml over about 168 hours of administration after a single-dose administration to a subject population, in particular containing buprenorphine in the area of release in an amount of less than 0.8 mg/cm² buprenorphine base or an equimolar amount of a pharmaceutically acceptable salt thereof.

According to one aspect, the invention relates to a transdermal therapeutic system comprising buprenorphine for the transdermal administration of buprenorphine selected from:

a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a nominal mean release rate of about 5 μg/hr over about 168 hours of administration; and
a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a nominal mean release rate of about 10 μg/hr over about 168 hours of administration; and
a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a nominal mean release rate of about 20 μg/hr over about 168 hours of administration; and
a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a nominal mean release rate of about 30 μg/hr over about 168 hours of administration; and
a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a nominal mean release rate of about 40 μg/hr over about 168 hours of administration, in particular containing buprenorphine in the area of release in an amount of less than 0.8 mg/cm² buprenorphine base or an equimolar amount of a pharmaceutically acceptable salt thereof.

According to one aspect, the invention relates to a set of transdermal therapeutic systems including at least two transdermal therapeutic systems selected from the first, second, third, fourth and fifth transdermal therapeutic system as described in the previous paragraphs.

According to one aspect, the invention relates to a method of treating pain in a patient by selecting for said patient the appropriate transdermal therapeutic system from the first, second, third, fourth and fifth transdermal therapeutic system as described in the previous paragraphs and subsequently applying said selected transdermal therapeutic system on the skin of said patient for 7 days.

According to one aspect, the invention relates to a set of two to five different transdermal therapeutic systems for the transdermal administration of buprenorphine selected from five different transdermal therapeutic systems, a first, a second, a third, a forth and a fifth transdermal therapeutic system, each of the five different transdermal therapeutic systems comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
   and optionally wherein the buprenorphine-containing self-adhesive layer structure contains said buprenorphine in an amount of less than 0.8 mg/cm² buprenorphine base or an equimolar amount of a pharmaceutically acceptable salt thereof, and wherein
   the first transdermal therapeutic system provides a size of said buprenorphine-containing matrix layer providing the area of release ranging from more than 4.8 cm² to about 8 cm²; and
   the second transdermal therapeutic system provides a size of said buprenorphine-containing matrix layer providing the area of release ranging from more than 9.5 cm² to about 15 cm²; and
   the third transdermal therapeutic system provides a size of said buprenorphine-containing matrix layer providing the area of release ranging from more than 19 cm² to about 30 cm²; and
   the fourth transdermal therapeutic system provides a size of said buprenorphine-containing matrix layer providing the area of release ranging from more than 28.5 cm² to about 45 cm²; and
   the fifth transdermal therapeutic system provides a size of said buprenorphine-containing matrix layer providing the area of release ranging from more than 38 cm² to about 60 cm², wherein the five different transdermal therapeutic systems have increasing areas of release from the first to the fifth transdermal therapeutic system.

According to one aspect, the invention relates to a method of treating pain in a patient by selecting for said patient the appropriate transdermal therapeutic system from the set of different transdermal therapeutic systems as described in the previous paragraph and subsequently applying said selected transdermal therapeutic system on the skin of said patient for 7 days.

According to one aspect, the invention relates to a transdermal therapeutic system selected from a set as described in the previous paragraphs for use in a method of treating pain in a patient by applying said selected transdermal therapeutic system for 7 days on the skin of the patient.

Within the meaning of this invention, the term “transdermal therapeutic system” (or TTS) refers to the entire individual unit that is applied to the skin of a patient, and which comprises the buprenorphine-containing self-adhesive layer structure and optionally an additional larger active agent-free self-adhesive layer structure on top of the buprenorphine-containing self-adhesive layer structure, which TTS provides the percutaneous delivery of the active buprenorphine to the patient. During storage, such a TTS is normally located on a redetachable protective layer from which it is removed immediately before application to the surface of the patient's skin. A TTS protected this way may be stored in a blister pack or a side sealed bag.

Within the meaning of this invention, the term “buprenorphine-containing self-adhesive layer structure” refers to the active agent-containing structure.

Within the meaning of this invention, the term “additional larger active agent-free self-adhesive layer structure” refers to a self-adhesive layer structure that is free of active agent and larger than the active agent-containing structure and providing additional area adhering to the skin, but no area of release of the active agent, and enhancing thereby the overall adhesive properties of the TTS.

Within the meaning of this invention, the term “buprenorphine-containing matrix layer” refers to the layer containing the active in a matrix-type structure of active in polymer or polymer-based adhesive, and providing the area of release of the active agent. During the storage of the TTS some of the active buprenorphine or some of the carboxylic acid may migrate from the buprenorphine-containing matrix layer into the skin contact layer. Thus the composition of the buprenorphine-containing matrix layer may change during storage. The “initial composition” refers to the composition before storage and thus before migration.

Within the meaning of this invention, the term “polymer base” refers to a composition containing from 75% to 100% of polymer based on the dry weight of the composition. The polymer base may contain 75% to 100% of one or more polymers. According to certain embodiments the polymer base is a polymer-based pressure-sensitive adhesive.

Within the meaning of this invention, “polymer-based pressure-sensitive adhesive” refers to a pressure-sensitive adhesive containing from 75% to 100% of said polymer based on the dry weight of the pressure-sensitive adhesive. According to certain embodiments the pressure-sensitive adhesive contains from 80% to 100% or from 85% to 100%, or from 90% to 100%, or from 95% to 100% of the polymer (e.g., polysiloxane) based on the dry weight of the pressure sensitive adhesive. A pressure-sensitive adhesive is in particular a material that adheres with finger pressure, is permanently tacky, exerts a strong holding force and should be removable from smooth surface without leaving a residue. Such polymer-based pressure-sensitive adhesives may e.g., comprise polysiloxane, polyacrylate or polyisobutylene. Polymer-based pressure-sensitive adhesives comprising polysiloxane or polyacrylate are preferred. Examples of useful pressure-sensitive adhesives comprising polysiloxane which are commercially available include the standard Bio-PSA series (7-4400, 7-4500 and 7-4600 series), the amine compatible (endcapped) Bio-PSA series (7-4100, 7-4200 and 7-4300 series), the Soft Skin Adhesives series (7-9800) and the Bio-PSA Hot Melt Adhesive manufactured by Dow Corning. Preferred pressure-sensitive adhesives comprising polysiloxane are heptane-solvated pressure-sensitive adhesives including BIO-PSA 7-4201, BIO-PSA 7-4301. A useful pressure-sensitive adhesive comprising polyacrylate which is commercially available is Duro Tak® 387 2051 from Henkel.

Within the meaning of this invention, the term “deposit” refers to a distinguishable, e.g., visually distinguishable, area within the polymer base, e.g., the polymer-based pressure-sensitive adhesive. Such deposits are e.g., droplets. Deposits that are visually distinguishable may be identified by use of a microscope.

Within the meaning of this invention, the term “skin contact layer” refers to the part of the TTS which is in direct contact with the skin of the patient during administration and is located in the buprenorphine-containing self-adhesive layer structure on top of the buprenorphine containing matrix layer. The sizes of the skin contact layer, the buprenorphine-containing matrix layer and the buprenorphine-containing self-adhesive layer structure are co-extensive and correspond to the area of release.

Within the meaning of this invention, the parameter “mean cumulative skin permeation rate” is provided in μg/cm²-hr and is calculated from the cumulative release as measured by in vitro experiments carried out with the Franz diffusion cell over the total time period of release, e.g., 168 hours, in μg/cm² divided by the hours corresponding to said total time period of release, e.g., 168 hours.

Within the meaning of this invention, the parameter “mean non-cumulative skin permeation rate” is provided in μg/cm²-hr and is calculated from the non-cumulative release of a certain sample interval as measured in a Franz diffusion cell in μg/cm² divided by the hours of said sample interval.

Within the meaning of this invention, the parameter “cumulative release” is provided in μg/cm² and relates to the total amount released over the total time period of release, e.g., 168 hours, as measured in a Franz diffusion cell. The value is a mean value of at least 3 experiments.

Within the meaning of this invention, the parameter “non-cumulative release” is provided in μg/cm² and relates to the amount released in a sample interval at certain elapsed time within the total time period of release, e.g., hour 16 of release corresponding to a sample interval of 8 hours from hour 8 to hour 16 of release within 168 hours of total time period of release, as measured in a Franz diffusion cell. The value is a mean value of at least 3 experiments.

Within the meaning of this invention, the parameter “mean release rate” refers to the mean release rate in μg/hr over the period of administration (e.g., 7 days) by which the active agent permeates through the human skin into the blood system and is based on the AUC obtained over said period of administration in a clinical study.

Within the meaning of this invention, the parameter “nominal mean release rate” refers to an assigned mean release rate determined by comparison with the commercial reference product BuTrans® which is applied for 7 days to the skin of the subjects and of which mean release rates are publicly available from the package insert. The corresponding known nominal mean release rate of the 25 cm² area of release BuTrans® reference TTS containing 20 mg buprenorphine is 20 μg/hr. The mean release rate is proportional to the size of the area of release of a TTS and may be used to distinguish TTSs by the dosage strength. The BuTrans® TTS with half the size (i.e. 12.5 cm² area of release) and containing 10 mg of buprenorphine provides the known nominal mean release rate of 10 μg/hr. The BuTrans® TTS with a size of 6.25 cm² area of release and containing 5 mg of buprenorphine provides the known nominal mean release rate of 5 μg/hr. Accordingly, it can be assumed that a corresponding TTS with a size of 50 cm² area of release and containing 40 mg of buprenorphine provides a nominal mean release rate of 40 μg/hr, and a corresponding TTS with a size of 37.5 cm² area of release and containing 30 mg of buprenorphine provides a nominal mean release rate of 30 μg/hr. The nominal mean release rates are assigned to the TTSs in accordance with the invention based on bioequivalence considerations by at least comparing the mean AUCt of the reference TTS BuTrans® with the mean AUCt of the TTSs in accordance with the invention obtained in the same clinical study.

Within the meaning of this invention, the meaning of “by applying the TTS for 7 days on the skin of said patient” corresponds to “by applying the TTS for about 168 hours on the skin of said patient” and refers to a once a week exchange mode or dosing regimen. Likewise, 4 days correspond to about 96 hours, 5 days correspond to about 120 hours and 6 days correspond to about 144 hours. The term “applying on the skin of a patient for a certain period of time” has the same meaning as “administration for a certain period of time”.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

If not indicated otherwise “%” refers to weight-%.

Within the meaning of this invention, the term “active”, “active agent”, and the like, as well as the term “buprenorphine” refers to buprenorphine base or a pharmaceutically acceptable salt thereof. Unless otherwise indicated the amounts of buprenorphine in the TTS relate to the amount of buprenorphine before administration of the TTS. The amounts of buprenorphine in the TTS after administration are referred to as residual amounts.

Within the meaning of this invention, values and ranges specifying the area of release and the amount of buprenorphine contained in the transdermal therapeutic system are mean values of at least 3 measurements.

Within the meaning of this invention the term “pharmacokinetic parameters” refers to parameters describing the blood plasma curve, e.g. Cmax, AUCt and AUCINF obtained in a clinical study, e.g. by single-dose administration of the active agent TTS, e.g. the buprenorphine base TTS to healthy human subjects. The pharmacokinetic parameters of the individual subjects are summarized using arithmetic and geometric means, e.g. a mean Cmax, a mean AUCt and a mean AUCINF, and additional statistics such as the respective standard deviations and standard errors, the minimum value, the maximum value, and the middle value when the list of values is ranked (Median). In the context of the present invention, pharmacokinetic parameters, e.g. the mean Cmax, the mean AUCt and the mean AUCINF refer to geometric mean values if not indicated otherwise. It cannot be precluded that the absolute mean values obtained for a certain TTS in a clinical study vary to a certain extend from study to study. To allow a comparison of absolute mean values between studies, a reference formulation, e.g. the commercial reference product BuTrans® or in the future any product based on the invention, may be used as internal standard. A comparison of the AUC per area of release, e.g. the mean AUCt per area of release of the respective reference product in the earlier and later study can be used to obtain a correction factor to take into account differences from study to study.

Clinical studies according to the present invention refer to studies performed in full compliance with the International Conference for Harmonization of Clinical Trials (ICH) and all applicable local Good Clinical Practices (GCP) and regulations.

Within the meaning of this invention, the term “healthy human subject” refers to a male or female subject with a body weight ranging from 55 kg to 100 kg and a body mass index (BMI) ranging from 18 to 29 and normal physiological parameters, such as blood pressure, etc. Healthy human subjects for the purposes of the present invention are selected according to inclusion and exclusion criteria which are based on and in accordance with recommendations of the ICH.

Within the meaning of this invention, the term “subject population” refers to at least ten individual healthy human subjects.

Within the meaning of this invention, the term “geometric mean” refers to the mean of the log transformed data backtransformed to the original scale.

Within the meaning of this invention, the term “arithmetic mean” refers to the sum of all values of observation divided by the total number of observations.

Within the meaning of this invention, the parameter “AUC” corresponds to the area under the plasma concentration-time curve. The AUC value is proportional to the amount of active agent absorbed into the blood circulation in total and is hence a measure for the bioavailability.

Within the meaning of this invention, the parameter “AUCt” is provided in pg·hr/ml and relates to the area under the plasma concentration-time curve from hour 0 to the last measurable plasma concentration and is calculated by the linear trapezoidal method.

Within the meaning of this invention, the parameter “mean AUCt per area of release” is provided in pg·hr/ml-cm² and is calculated from the geometric mean AUCt as determined for a certain TTS in pg·hr/ml divided by the area of release of said TTS.

Within the meaning of this invention, the parameter “AUCINF” is provided in pg·hr/ml and relates to the area under the plasma concentration-time curve extrapolated to infinity and is calculated using the formula:

$$AUCINF = AUCt + \frac{CLast}{LambdaZ}$$

where CLast is the last measurable plasma concentration and LambdaZ is the apparent terminal phase rate constant.

Within the meaning of this invention, the parameter “Cmax” is provided in pg/ml and and relates to the maximum observed blood plasma concentration of the active agent.

Within the meaning of this invention, the parameter “tmax” is provided in hr and relates to the time point at which the Cmax value is reached. In other words, tmax is the time point of the maximum observed plasma concentration.

Within the meaning of this invention, the parameter “LambdaZ” is provided in 1/hr and relates to the apparent terminal phase rate constant, where LambdaZ is the magnitude of the slope of the linear regression of the log concentration versus time profile during the terminal phase.

Within the meaning of this invention, the parameter “t½Z” is provided in hr and relates to the apparent plasma terminal phase half-life and is commonly determined as t½Z=(ln 2)/LambdaZ.

Within the meaning of this invention, the term “mean plasma concentration” is provided in pg/ml and is a mean of the individual plasma concentrations of active agent, e.g. buprenorphine base, at each point in time.

### BRIEF DESCRIPTION OF THE DRAWINGS

FIG. 1 depicts the mean non-cumulative skin permeation rate for Examples 1 to 3 and Norspan®.

FIG. 2 depicts the mean non-cumulative skin permeation rate of the transdermal therapeutic systems. The area of release of the transdermal therapeutic systems according to Examples 1 to 3 being 15 cm² and the area of release for Norspan® being 25 cm². The amount of buprenorphine base for Examples 1 to 3 being 6.75 mg and the amount of buprenorphine base for Norspan® being 20 mg.

FIG. 3 depicts the mean plasma concentration for Example 1 and BuTrans®. The area of release for Example 1 being 15 cm² and the area of release for BuTrans® being 25 cm². The amount of buprenorphine base for Example 1 being 6.75 mg and the amount of buprenorphine base for BuTrans® being 20 mg.

### DETAILED DESCRIPTION

### TTS Structure

According to the invention wherein the structure is concerned, the TTS for the transdermal administration of buprenorphine comprises a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive.

According to an aspect of the invention, the TTS for the transdermal administration of buprenorphine base comprises a buprenorphine base-containing self-adhesive layer structure comprising
A) a buprenorphine base-impermeable backing layer, and
B) a buprenorphine base-containing matrix layer on said buprenorphine base-impermeable backing layer, the matrix layer comprising
   a) a polymer-based pressure-sensitive adhesive comprising polysiloxane, b) buprenorphine base, and
   c) levulinic acid, in an amount sufficient so that said buprenorphine base is solubilized therein to form a mixture, and the levulinic acid buprenorphine base mixture forms dispersed deposits in the said pressure-sensitive adhesive, and
C) a skin contact layer on said buprenorphine base-containing matrix layer comprising a polymer-based pressure-sensitive adhesive comprising polyacrylate.

According to certain embodiments of the invention, the TTS comprises in addition to the buprenorphine-containing self-adhesive layer structure attached thereto a larger active agent-free self-adhesive layer structure, e.g., a peripheral adhesive or overlying adhesive, for enhancing the adhesive properties of the overall transdermal therapeutic system. The area of said second active agent agent-free self-adhesive layer structure adds to the overall size of the TTS but does not add to the area of release. Said active agent-free self-adhesive layer structure comprises also a backing layer, e.g., beige colored, and an active agent free pressure-sensitive adhesive layer of polymer-based pressure-sensitive adhesive, e.g., comprising polyacrylate, polyisobutylene or polysiloxane. Polyacrylate-based pressure-sensitive adhesives are preferred for the active agent free pressure-sensitive adhesive layer, in particular pressure-sensitive adhesives comprising an acrylate-vinylacetate polymer, e.g., such as those available from Henkel under the tradename Duro Tak®, e.g., Duro Tak® 387 2051. Such pressure-sensitive adhesives are provided in an organic solution of ethyl acetate and heptane or only one of these solvents. Such pressure-sensitive adhesives provide a 180° Peel at 20 minutes of at least about 20 N/25 mm, and at 24 minutes of at least about 25 N/25 cm, and at one week of at least about 30 N/25 mm and a Loop tack of at least 15 N/25 mm², or of at least 20 N/25 mm², or of at least 22 N/25 mm².

### Active Agent

The TTS according to the invention comprises buprenorphine base or an equimolar amount of a pharmaceutically acceptable salt thereof. Pharmaceutically acceptable salts may be selected from those known in the art, such as the hydrochloride, sulphate, phosphate, tartrate, maleinate, oxalate, acetate and lactate salts. According to a preferred embodiment of the invention the active agent is buprenorphine base.

The amount of buprenorphine contained in the TTS may vary from about 1 mg to about 32 mg of buprenorphine base or an equimolar amount of a pharmaceutically acceptable salt thereof. According to certain embodiments, the TTS contains according to five different dosage strengths from about 1 mg to about 4 mg, or about 2.5 mg, or from about 3.5 mg to about 8 mg, or about 5 mg, or from about 6.5 mg to about 16 mg, or about 10 mg, or from about 11.5 mg to about 24 mg, or about 15 mg or from about 15 mg to about 32 mg, or about 20 mg of buprenorphine base or a an equimolar amount of a pharmaceutically acceptable salt thereof.

The amount of buprenorphine contained in the buprenorphine-containing self-adhesive layer structure may be less than 0.8 mg/cm², or may vary from about 0.2 mg/cm² to less than 0.8 mg/cm² buprenorphine base or an equimolar amount of a pharmaceutically acceptable salt thereof. According to certain embodiments, the buprenorphine-containing self-adhesive layer structure contains less than 0.7 mg/cm², or less than 0.6 mg/cm², or less than 0.55 mg/cm², or less than 0.5 mg/cm², or contains from about 0.2 mg/cm² to about 0.7 mg/cm², or from about 0.2 mg/cm² to about 0.6 mg/cm², or from about 0.2 mg/cm² to less than 0.55 mg/cm², or from about 0.2 mg/cm² to about 0.5 mg/cm², or from about 0.3 mg/cm² to about 0.5 mg/cm², or from about 0.4 mg/cm² to about 0.5 mg/cm², or about 0.45 mg/cm² buprenorphine base or an equimolar amount of a pharmaceutically acceptable salt thereof. Based on the dry weight of the initial composition of the buprenorphine-containing matrix layer, more than 4%, or more than 5%, or more than 6%, or more than 7%, or from about 5% to about 20%, or from about 6% to about 20%, or from about 7% to about 15%, or about 7.5% buprenorphine base or equimolar amounts of pharmaceutically acceptable salts are contained in the buprenorphine-containing self-adhesive layer structure.

### Polymer Base/Pressure-Sensitive Adhesive

In accordance with the invention, a polymer base is used to form the matrix containing the active buprenorphine. The polymer base contains from 75% to 100% of polymer. The polymer base may contain 75% to 100% of one or more polymers.

According to certain preferred embodiments, the polymer base is a pressure-sensitive adhesive. Such polymer-based pressure-sensitive adhesives may e.g., comprise polysiloxane or polyisobutylene. For the present invention polysiloxane-based pressure-sensitive adhesives are preferred for the buprenorphine-containing matrix layer. Such polysiloxane adhesives need, unlike other organic pressures-sensitive adhesives, no additives like antioxidants, stabilizers, plasticizers, catalysts or other potentially extractable ingredients. These pressure-sensitive adhesives provide for suitable tack for quick bonding to various skin types, including wet skin, suitable adhesive and cohesive qualities, long lasting adhesion to the skin of up to 7 days, a high degree of flexibility, a permeability to moisture, and compatibility to many actives and film-substrates. It is possible to provide them with sufficient amine resistance and therefore enhanced stability in the presence of amines. Such pressure-sensitive adhesives are based on a resin-in-polymer concept wherein, by condensation reaction of silanol end blocked polydimethylsiloxane with a silica resin, a polysiloxane is prepared which for amine stability the residual silanol functionality is additionally capped with trimethylsiloxy groups. The dimethiconol content contributes to the viscous component of the visco-elastic behavior, and impacts the wetting and the spreadability properties of the adhesive. The resin acts as a tackifying and reinforcing agent, and participates in the elastic component. The correct balance between dimethiconol and resin provides for the correct adhesive properties.

The preferred pressure-sensitive adhesives comprising polysiloxane in accordance with the invention are characterized by a solution viscosity at 25° C. and 60% solids content in heptane of more than about 150 mPa s, or from about 200 mPa s to about 700 mPa s, in particular from about 350 mPa s to about 600 mPa s, more preferred from about 480 mPa s to about 550 mPa s, or most preferred of about 500 mPa s or alternatively from about 400 mPa s to about 480 mPa s, or most preferred of about 450 mPa s. Theses may also be characterized by a complex viscosity at 0.01 rad/s at 30° C. of less than about 1×10⁹ Poise or from about 1×10⁵ to about 9×10⁸ Poise, or more preferred from about 1×10⁵ to about 1×10⁷ Poise, or most preferred about 5×10⁶ Poise or alternatively more preferred from about 2×10⁷ to about 9×10⁸ Poise, or most preferred about 1×10⁸ Poise.

The above described adhesives for the buprenorphine-containing matrix layer may also be used for the skin contact layer, and in this case polysiloxane-based pressure-sensitive adhesives are preferred. The adhesive strength of the polysiloxane may be sufficient for the desired skin contact. In certain embodiments of the invention a plasticizer or a tackifying agent is incorporated into the formulation to improve the adhesive characteristics of the pressure-sensitive adhesive in the skin contact layer. It may be advantageous in an individual case to improve the tack by adding small amounts of tackifiers such as polyterpenes, rosin derivatives, or silicone oils. In preferred embodiments, the tackifying agent is a silicone oil (e.g., 360 Medical Fluid, available from Dow Corning Corporation, Midland, Mich.).

According to certain other embodiments the adhesives in the buprenorphine-containing matrix layer and the skin contact layer are different, and the adhesive in the skin contact layer is a pressure-sensitive adhesive based on polyacrylate, in particular a pressure-sensitive adhesives based on an acrylate-vinylacetate polymer prepared from 2-ethylhexyl acrylate, vinylacetate and 2-hydroxyethyl acrylate.

The pressure-sensitive adhesives are supplied and used in solvents like heptane, ethyl acetate or other volatile silicone fluids. For the pressure-sensitive adhesives comprising polysiloxane heptane is preferred and the solids content is usually between 60 and 80%. For the pressure-sensitive adhesives comprising polyacrylate ethyl acetate is preferred and the solids content is usually between 40 and 80%.

Suitable pressure-sensitive adhesives comprising polysiloxane may be obtained from Dow Corning® BIO-PSA Standard Silicone Adhesives. Preferred are the BIO-PSA 7 4301 and BIO-PSA 7 4201 Silicone Adhesives. According to certain embodiments BIO-PSA 7 4301 is preferred and according to certain other embodiments BIO-PSA 7 4201 is preferred. BIO-PSA 4201 has a solution viscosity at 25° C. and about 60% solids content in heptane of 450 mPa s and a complex viscosity at 0.01 rad/s at 30° C. of 1×10⁸ Poise. BIO-PSA 4301 has a solution viscosity at 25° C. and about 60% solids content in heptane of 500 mPa s and a complex viscosity at 0.01 rad/s at 30° C. of 5×10⁶ Poise.

Suitable pressure-sensitive adhesives comprising polyacrylate may be obtained from Henkel under the tradename Duro Tak®, e.g., Duro Tak® 387 2051. Such pressure-sensitive adhesives are provided in an organic solution of ethyl acetate and heptane or only one of these solvents. Such pressure-sensitive adhesives provide a 180° Peel at 20 minutes of at least about 20 N/25 mm, and at 24 minutes of at least about 25 N/25 cm, and at one week of at least about 30 N/25 mm and a Loop tack of at least 15 N/25 mm², or of at least 20 N/25 mm², or of at least 22 N/25 mm².

The adhesive in the active agent-free pressure-sensitive adhesive layer may be a pressure-sensitive adhesive comprising polysiloxane, polyacrylate or polyisobutylene, and polyacrylate based pressure-sensitive adhesives are preferred, in particular pressure-sensitive adhesives based on an acrylate-vinylacetate polymer prepared from 2-ethylhexyl acrylate, vinylacetate and 2-hydroxyethyl acrylate.

The buprenorphine-containing matrix layer of the TTS according to the invention may further comprise in addition to the above mentioned ingredients a), b) and c), namely a polymer-base, the buprenorphine and the carboxylic acid selected from the group of oleic acid, linoleic acid, linolenic acid and levulinic acid as described herein, other various excipients or additives, for example from the group of solubilizers, fillers, tackifiers, substances which influence the barrier properties of the stratum corneum in the sense of increasing the active agent permeability, pH regulators, and preservatives.

Substances which influence the barrier properties of the stratum corneum in the sense of increasing the active agent permeability are known to the skilled worker and the substance appropriate for the respective active agents must—if necessary—be found by means of permeation studies. Some examples are polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin; polar solvents such as dimethyldecylphosphoxide, methyloctylsulfoxide, dimethyllaurylamine, dodecylpyrrolidone, isosorbitol, dimethylacetonide, dimethylsulfoxide, decylmethylsulfoxide, and dimethylformamide; salicylic acid; amino acids; benzyl nicotinate; and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate. The TTS of the invention may additionally comprise according to certain embodiments in which the buprenorphine-containing matrix layer comprises a) the polymer-based pressure-sensitive adhesive, b) the buprenorphine and c) levulinic acid or linolenic acid or mixtures of both as the carboxylic acid as described herein, oleic and linoleic acids as substances influencing the barrier properties of the stratum corneum in the sense of increasing the active agent permeability.

Such substances as described in the previous paragraph may be included in a TTS and may be present in an amount of about 1% to about 10% by weight. In a preferred embodiment of the present invention such additional substances are however not necessary. According to an embodiment of the invention the TTS does not comprise such additional substances as mentioned in the previous paragraph.

In addition to the carboxylic acid selected from oleic acid, linoleic acid, linolenic acid, levulinic acid, the solubility of the drug can be further altered by the optional addition of an agent that increases the solubility of drug or inhibits drug crystallization in the transdermal composition, such as polyvinylpyrrolidone, vinyl acetate/vinylpyrrolidone copolymer and cellulose derivatives.

Viscosity-increasing substances are preferably used in conjunction with an active agent solution. Suitable substances for increasing the viscosity of the active agent solution are, for example, cellulose derivatives such as ethylcellulose, hydroxylpropylcellulose and high molecular mass polyacrylic acids and/or their salts and/or their derivatives such as esters.

Fillers such as silica gels, titanium dioxide and zinc oxide may be used in conjunction with the polymer in order to influence certain physical parameters, such as cohesion and bond strength, in the desired way.

### Buprenorphine-Containing Self-Adhesive Layer Structure

The buprenorphine-containing self-adhesive layer structure according to the invention comprises a buprenorphine-impermeable backing layer, a buprenorphine-containing matrix layer on said backing layer, and a skin contact layer on said buprenorphine-containing matrix layer. In a preferred embodiment, the buprenorphine-containing self-adhesive layer structure consists of these three elements.

The buprenorphine-containing matrix layer may be coated at any dry weight, but is preferably coated at a dry weight of less than 8 mg/cm² (less than 80 g/m²), but is preferably coated at a dry weight of less than 7 mg/cm² (less than 70 g/m²), or of up to 6 mg/cm² (up to 60 g/m²), or of less than 6 mg/cm² (less than 60 g/m²), or ranging from about 3 mg/cm² (about 30 g/m²) to less than 8 mg/cm² (less than 80 g/m²), or from about 4 mg/cm² (about 40 g/m²) to less than 8 mg/cm² (less than 80 g/m²), or from about 5 mg/cm² (about 50 g/m²) to about 7 mg/cm² (about 70 g/m²), or from about 5.5 mg/cm² (about 55 g/m²) to about 6.5 mg/cm² (about 65 g/m²), or is specifically about 6 mg/cm² (about 60 g/m²).

The size of the buprenorphine-containing matrix layer which provides the area of release may range from more than 4.8 cm² to about 60 cm². According to certain embodiments, the area of release ranges according to five different dosages from more than 4.8 cm² to about 8 cm², or is about 5.5 cm², or ranges from more than 9.5 cm² to about 15 cm², or is about 11.25 cm², or ranges from more than 19 cm² to about 30 cm², or is about 22.5 cm², or ranges from more than 28.5 cm² to about 45 cm², or is about 33.75 cm², or ranges from more than 38 cm² to about 60 cm², or is about 45 cm².

The skin contact layer may be coated at any dry weight, but is preferably coated at a dry weight of less than 6 mg/cm² (less than 60 g/m²), or of less than 5 mg/cm² (less than 50 g/m²), or of less than 4 mg/cm² (less than 40 g/m²), or ranging from about 1 mg/cm² (about 10 g/m²) to less than 6 mg/cm² (about 60 g/m²), or from about 1 mg/cm² (about 10 g/m²) to about 5 mg/cm² (about 50 g/m²), or from about 1 mg/cm² (about 10 g/m²) to about 4 mg/cm² (about 40 g/m²), or from about 1 mg/cm² (about 10 g/m²) to about 3 mg/cm² (about 30 g/m²), or from about 1.5 mg/cm² (about 15 g/m²) to about 2.5 mg/cm² (about 25 g/m²), or is specifically about 2 mg/cm² (about 20 g/m²).

The buprenorphine-containing self-adhesive layer structure preferably contains buprenorphine base, but may contain equimolar amounts of pharmaceutically acceptable salts. According to the invention preferably more than 4%, or more than 5%, or more than 6%, or more than 7%, or from about 5% to about 20%, or from about 6% to about 20%, or from about 7% to about 15% buprenorphine base or equimolar amounts of pharmaceutically acceptable salts based on the dry weight of the initial composition of the buprenorphine-containing matrix layer are contained in the buprenorphine-containing self-adhesive layer structure. In a specific embodiment, about 7.5% buprenorphine base is contained in the buprenorphine-containing self-adhesive layer structure.

The buprenorphine-containing self-adhesive layer structure in particular contains less than 0.8 mg/cm², or less than 0.7 mg/cm², or less than 0.6 mg/cm², or less than 0.55 mg/cm², or less than 0.5 mg/cm², or from about 0.2 mg/cm² to less than 0.8 mg/cm², or from about 0.2 mg/cm² to about 0.7 mg/cm², or from about 0.2 mg/cm² to about 0.6 mg/cm², or from about 0.2 mg/cm² to less than 0.55 mg/cm², or from about 0.2 mg/cm² to about 0.5 mg/cm², or from about 0.3 mg/cm² to about 0.5 mg/cm², or from about 0.4 mg/cm² to about 0.5 mg/cm² buprenorphine base or contains about 0.45 mg/cm² buprenorphine base. The TTS may also contain equimolar amounts of pharmaceutically acceptable salts.

In order to provide the desired delivery rate of buprenorphine, a carboxyclic acid is present. The carboxylic acid may be selected from the group consisting of oleic acid, linoleic acid, linolenic acid, levulinic acid and mixtures thereof, wherein levulinic acid is preferred. The buprenorphine is in mixture with, e.g., dissolved in, the carboxylic acid, e.g., the levulinic acid, and this mixture, e.g., solution, is dispersed in the form of small deposits, e.g., droplets, in the matrix layer. Buprenorphine, with its known physicochemical properties, namely its poor solubility, its comparatively high melting point of 216° C., and its high molecular weight, tends readily towards crystallization. For this reason, a solubilizer with at least one acidic group is used in order to prevent the buprenorphine from crystallizing during the storage of the pharmaceutical form. Buprenorphine and levulinic acid have an extremely low solubility in polysiloxanes. As a consequence of this, it is possible to solubilize buprenorphine in levulinic acid and to disperse this mixture in the form of small deposits in a matrix layer prepared on the basis of polysiloxanes as described herein.

Levulinic acid is sparingly soluble in the organic solvents of the adhesives. Consequently, the liquid mixture of buprenorphine and levulinic acid can be dispersed in the solution of the adhesive, with the dispersion being retained following removal of the solvent. In a matrix layer of this kind, the solubility of the buprenorphine is dependent virtually only on the amount of the levulinic acid.

The amount of the dispersed mixture of buprenorphine, e.g., buprenorphine base, and the carboxylic acid, e.g., levulinic acid, can be up to about 40% by weight, it being preferred not to exceed about 25% or about 20% by weight and ranges from about 15% to about 25%, or from about 15% to about 20%, or from about 17% to about 20%. The deposit, e.g., droplet, size (diameter) itself ought preferably not to exceed about 150 μm, or ranges from about 1 to about 150 μm, preferably from about 1 to about 50 μm, or from about 5 to about 50 μm, or from about 1 to about 25 μm or from about 5 to about 25 μm. The preferred size is dependent, furthermore, on the thickness of the matrix layer.

Since the carboxylic acid, e.g., the levulinic acid, can likewise be absorbed through the skin, the amount in the TTS becomes less as the time of application elapses, and leads to a reduction of the solubility of buprenorphine. As a result, the decrease in the thermodynamic activity of buprenorphine due to depletion is compensated by the reduced drug solubility in the buprenorphine/levulinic acid deposits.

According to the invention the buprenorphine-containing self-adhesive layer structure contains more than 4%, or more than 5%, or more than 6%, or more than 7%, or more than 8%, or 9% or more, or more than 9%, or from about 5% to about 20%, or from about 6% to about 20%, or from about 7% to about 15%, or from about 8% to about 15%, or from about 9% to about 15% carboxylic acid, or about 9%, or about 10% carboxylic acid e.g., levulinic acid based on the dry weight of the initial composition of the buprenorphine-containing matrix layer. In a specific embodiment the buprenorphine-containing self-adhesive layer structure contains from about 5% to about 20% levulinic acid, or from about 6% to about 20%, or from about 7% to about 15%, or from about 8% to about 15%, or from about 9% to about 15% levulinic acid, or about 9%, or about 10% levulinic acid based on the dry weight of the initial composition of the buprenorphine-containing matrix layer. According to a specific embodiment the buprenorphine-containing self-adhesive layer structure contains the same %-amount of levulinic acid and buprenorphine base or equimolar amounts of pharmaceutically acceptable salts. According to another specific embodiment, the buprenorphine-containing self-adhesive layer structure contains less %-amount of buprenorphine base or equimolar amounts of pharmaceutically acceptable salts than it contains %-amount of levulinic acid.

According to a specific embodiment, the buprenorphine-containing self-adhesive layer structure contains from about 5% to about 20% buprenorphine base and from about 5% to about 20% levulinic acid based on the dry weight of the initial composition of the buprenorphine-containing matrix layer, or from about 7% to about 15% buprenorphine base and from about 9% to about 15% levulinic acid based on the dry weight of the initial composition of the buprenorphine-containing matrix layer.

According to a certain embodiment, the buprenorphine-containing matrix layer is coated at a dry weight of from about 5 mg/cm² (about 50 g/m²) to about 7 mg/cm² (about 70 g/m²), or from about 5.5 mg/cm² (about 55 g/m²) to about 6.5 mg/cm² (about 65 g/m²), or is about 6 mg/cm² (about 60 g/m²), and the buprenorphine-containing self-adhesive layer structure contains from about 6% to about 20%, or from about 7% to about 15%, or about 7.5% buprenorphine base and from about 7% to about 15%, or from about 8% to about 15%, or about 9% levulinic acid based on the dry weight of the initial composition of the buprenorphine-containing matrix layer. In a specific embodiment the buprenorphine-containing matrix layer is coated at a dry weight of about 6 mg/cm² and the buprenorphine-containing self-adhesive layer structure contains about 7.5% buprenorphine base and about 9% levulinic acid based on the dry weight of the initial composition of the buprenorphine-containing matrix layer.

According to a certain other embodiment, the buprenorphine-containing matrix layer being coated at a dry weight of from about 5 mg/cm² (about 50 g/m²) to about 7 mg/cm² (about 70 g/m²), or from about 5.5 mg/cm² (about 55 g/m²) to about 6.5 mg/cm² (about 65 g/m²), or is about 6 mg/cm² (about 60 g/m²), and the buprenorphine-containing self-adhesive layer structure contains from about 6% to about 20%, or from about 7% to about 15%, or about 7.5% buprenorphine base and from about 8% to about 15%, or from about 9% to about 15%, or about 10% levulinic acid based on the dry weight of the initial composition of the buprenorphine-containing matrix layer. In a specific embodiment the buprenorphine-containing matrix layer is coated at a dry weight of about 6 mg/cm² and the buprenorphine-containing self-adhesive layer structure contains about 7.5% buprenorphine base and about 10% levulinic acid based on the dry weight of the initial composition of the buprenorphine-containing matrix layer.

According to a certain embodiment of the invention, the polymer base in the buprenorphine-containing matrix layer is a polymer-based pressure-sensitive adhesive comprising polysiloxane or polyisobutylene. According to a specific embodiment the adhesive in the buprenorphine-containing matrix layer is an amine-resistant pressure-sensitive adhesive comprising polysiloxane wherein the polysiloxane is a product of the condensation reaction of silanol endblocked polydimethylsiloxane with a silica resin and the residual silanol functionality is capped with trimethylsiloxy groups and characterized by a solution viscosity at 25° C. and about 60% solids content in heptanes of about 500 mPa s or of about 450 mPa s, and the buprenorphine-containing matrix layer is coated at a dry weight of about 6 mg/cm² and the buprenorphine-containing self-adhesive layer structure contains about 7.5% buprenorphine base and about 9% or 10% levulinic acid based on the dry weight of the initial composition of the buprenorphine-containing matrix layer. The buprenorphine-containing matrix layer and the skin contact layer may contain the same or different pressure-sensitive adhesives.

According to a certain embodiment of the invention, the adhesive in the buprenorphine-containing matrix layer and the adhesive in the skin contact layer are different, and the adhesive in the skin contact layer is a pressure-sensitive adhesive comprising polyacrylate. According to a specific embodiment the adhesive in the skin contact layer is a pressure-sensitive adhesive comprising polyacrylate and the buprenorphine-containing matrix layer is a polymer-based pressure-sensitive adhesive comprising polysiloxane and is coated at a dry weight of about 6 mg/cm² and the buprenorphine-containing self-adhesive layer structure contains preferably about 7.5% buprenorphine base and about 9% or 10% levulinic acid based on the dry weight of the initial composition of the buprenorphine-containing matrix layer.

According to certain embodiments, the TTS contains from about 1 mg to about 32 mg of buprenorphine base or an equimolar amount of a pharmaceutically acceptable salt thereof. Considering five different increasing dosage strengths, the TTS in specific cases preferably contains
a) from about 1 mg to about 4 mg, preferably from about 1 mg to about 3.5 mg, more preferably from about 1 mg to about 3 mg, or about 2.5 mg buprenorphine base or an equimolar amount of a pharmaceutically acceptable salt thereof, or
b) from about 3.5 mg to about 8 mg, preferably from about 3.5 mg to about 7 mg, more preferably from about 3.5 mg to about 6 mg, or about 5 mg buprenorphine base or an equimolar amount of a pharmaceutically acceptable salt thereof, or
c) from about 6.5 mg to about 16 mg, preferably from about 6.5 mg to about 14 mg, more preferably from about 6.5 mg to about 12 mg, or about 10 mg buprenorphine base or an equimolar amount of a pharmaceutically acceptable salt thereof, or
d) from about 11.5 mg to about 24 mg, preferably from about 11.5 mg to about 21 mg, more preferably from about 12.5 mg to about 18 mg, or about 15 mg buprenorphine base or an equimolar amount of a pharmaceutically acceptable salt thereof, or
e) from about 15 mg to about 32 mg, preferably from about 15 mg to about 28 mg, more preferably from about 18.5 mg to about 24 mg, or about 20 mg of buprenorphine base or an equimolar amount of a pharmaceutically acceptable salt thereof.

Correspondingly the area of release ranges from more than 4.8 cm² to about 60 cm² and with respect to the five specific preferred dosage strengths a) to e)
a) ranges from more than 4.8 cm² to about 8 cm², preferably from about 5 cm² to about 7 cm², more preferably from about 5 cm² to about 6 cm², or is about 5.5 cm², or
b) ranges from more than 9.5 cm² to about 15 cm², preferably from about 10 cm² to about 13 cm², more preferably from about 10 cm² to about 12 cm², or is about 11.25 cm², or
c) ranges from more than 19 cm² to about 30 cm², preferably from about 20 cm² to about 26 cm², more preferably from about 20 cm² to about 24 cm², or is about 22.5 cm², or
d) ranges from more than 28.5 cm² to about 45 cm², preferably from about 30 cm² to about 39 cm², more preferably from about 30 cm² to about 36 cm², or is about 33.75 cm², or
e) ranges from more than 38 cm² to about 60 cm², preferably or from about 40 cm² to about 52 cm², more preferably from about 40 cm² to about 48 cm², or is about 45 cm².

In such embodiments the buprenorphine-containing matrix layer preferably comprises a pressure-sensitive adhesive comprising polysiloxane and is coated preferably at a dry weight of about 6 mg/cm², the skin contact layer preferably comprises a pressure-sensitive adhesive comprising polyacrylate, and the buprenorphine-containing self-adhesive layer structure preferably contains about 7.5% buprenorphine base based on the dry weight of the initial composition of the buprenorphine-containing matrix layer.

According to certain preferred embodiments, the TTS contains with respect to five dosage strengths a) to e) the following amounts of buprenorphine base or an equimolar amount of a pharmaceutically acceptable salt thereof and provides the following corresponding area of release ranges:

a)

| | more than 4.8 cm² | about 5 cm² to | about 5 cm² to |
|---|---|---|---|
| a) | to about 8 cm² | about 7 cm² | about 6 cm² |
| about 1 mg to | X | X | X |
| about 4 mg | | | |
| about 1 mg to | X | X | X |
| about 3.5 mg | | | |
| about 1 mg to | X | X | X |
| about 3 mg | | | |

b)

| | more than 9.5 cm² | about 10 cm² to | about 10 cm² to |
|---|---|---|---|
| b) | to about 15 cm² | about 13 cm² | about 12 cm² |
| about 3.5 mg | X | X | X |
| to about 8 mg | | | |
| about 3.5 mg | X | X | X |
| to about 7 mg | | | |
| about 3.5 mg | X | X | X |
| to about 6 mg | | | |

c)

| | more than 19 cm² | about 20 cm² to | about 20 cm² to |
|---|---|---|---|
| c) | to about 30 cm² | about 26 cm² | about 24 cm² |
| about 6.5 mg | X | X | X |
| to about 16 mg | | | |
| about 6.5 mg | X | X | X |
| to about 14 mg | | | |
| about 6.5 mg | X | X | X |
| to about 12 mg | | | |

d)

| | more than 28.5 cm² | about 30 cm² to | about 30 cm² to |
|---|---|---|---|
| d) | to about 45 cm² | about 39 cm² | about 36 cm² |
| about 11.5 mg | X | X | X |
| to about 24 mg | | | |
| about 11.5 mg | X | X | X |
| to about 21 mg | | | |
| about 12.5 mg | X | X | X |
| to about 18 mg | | | |

e)

| | more than 38 cm² | about 40 cm² to | about 40 cm² to |
|---|---|---|---|
| e) | to about 60 cm² | about 52 cm² | about 48 cm² |
| about 15 mg to | X | X | X |
| about 32 mg | | | |
| about 15 mg to | X | X | X |
| about 28 mg | | | |
| about 18.5 mg | X | X | X |
| to about 24 mg | | | |

### Set of Transdermal Therapeutic Systems

For the treatment of pain a patient needs to be titrated to the individual dose of buprenorphine to adequately control the pain. In order to meet the individual requirements, five different dosage strengths are provided in accordance with the invention.

According to one aspect, the invention relates to a set of two (first and second, or second and third, or third and fourth, or fourth and fifth TTS, or any other combination of two of the five different dosage strengths), three (first to third, or second to fourth or third to fifth TTS, or any other combination of three of the five different dosage strengths), four (first to fourth or second to fifth TTS, or any other combination of four of the five different dosage strengths) or five (first to fifth TTS) different transdermal therapeutic systems in accordance with the invention for the transdermal administration of buprenorphine for 7 days selected from:

a first transdermal therapeutic system providing a size of said buprenorphine-containing matrix layer providing the area of release ranging from more than 4.8 cm² to about 8 cm²;
a second transdermal therapeutic system providing the area of release ranging from more than 9.5 cm² to about 15 cm²; and
a third transdermal therapeutic system providing a size of said buprenorphine-containing matrix layer providing the area of release ranging from more than 19 cm² to about 30 cm²; and
a fourth transdermal therapeutic system providing the area of release ranging from more than 28.5 cm² to about 45 cm²; and
a fifth transdermal therapeutic system providing a size of said buprenorphine-containing matrix layer providing the area of release ranging from more than 38 cm² to about 60 cm², wherein the five different transdermal therapeutic systems have increasing areas of release from the first to the fifth transdermal therapeutic system.

According to a certain embodiment of the invention, the set of different transdermal therapeutic systems described in the previous paragraph comprises:

the first transdermal therapeutic system containing an amount of said buprenorphine ranging from about 1 mg to about 4 mg buprenorphine base or an equimolar amount of a pharmaceutically acceptable salt thereof and providing a size of said buprenorphine-containing matrix layer providing the area of release ranging from more than 4.8 cm² to about 8 cm²;
the second transdermal therapeutic system containing an amount of said buprenorphine ranging from about 3.5 mg to about 8 mg buprenorphine base or an equimolar amount of a pharmaceutically acceptable salt thereof and providing a size of said buprenorphine-containing matrix layer providing the area of release ranging from more than 9.5 cm² to about 15 cm²; and
the third transdermal therapeutic system containing an amount of said buprenorphine ranging from about 6.5 mg to about 16 mg buprenorphine base or an equimolar amount of a pharmaceutically acceptable salt thereof and providing a size of said buprenorphine-containing matrix layer providing the area of release ranging from more than 19 cm² to about 30 cm²; and
the fourth transdermal therapeutic system containing an amount of said buprenorphine ranging from about 11.5 mg to about 24 mg buprenorphine base or an equimolar amount of a pharmaceutically acceptable salt thereof and providing a size of said buprenorphine-containing matrix layer providing the area of release ranging from more than 28.5 cm² to about 45 cm²; and
the fifth transdermal therapeutic system containing an amount of said buprenorphine ranging from about 15 mg to about 32 mg buprenorphine base or an equimolar amount of a pharmaceutically acceptable salt thereof and providing a size of said buprenorphine-containing matrix layer providing the area of release ranging from more than 38 cm² to about 60 cm², wherein the five different transdermal therapeutic systems have increasing areas of release and increasing amounts of buprenorphine from the first to the fifth transdermal therapeutic system.

According to a certain embodiment of the invention, the set of different transdermal therapeutic systems described in the previous paragraph comprises:

the first transdermal therapeutic system providing a size of said buprenorphine-containing matrix layer providing the area of release ranging from about 5 cm² to about 7 cm²;
the second transdermal therapeutic system providing a size of said buprenorphine-containing matrix layer providing the area of release ranging from about 10 cm² to about 13 cm²; and
the third transdermal therapeutic system providing a size of said buprenorphine-containing matrix layer providing the area of release ranging from about 20 cm² to about 26 cm²; and
the fourth transdermal therapeutic system providing a size of said buprenorphine-containing matrix layer providing the area of release ranging from about 30 cm² to about 39 cm²; and
the fifth transdermal therapeutic system providing a size of said buprenorphine-containing matrix layer providing the area of release ranging from about 40 cm² to about 52 cm², wherein the five different transdermal therapeutic systems have increasing areas of release from the first to the fifth transdermal therapeutic system.

According to a certain embodiment of the invention, the set of different transdermal therapeutic systems described in the previous paragraph comprises:

the first transdermal therapeutic system containing an amount of said buprenorphine ranging from about 1 mg to about 3.5 mg buprenorphine base or an equimolar amount of a pharmaceutically acceptable salt thereof and providing a size of said buprenorphine-containing matrix layer providing the area of release ranging from about 5 cm² to about 7 cm²;
the second transdermal therapeutic system containing an amount of said buprenorphine ranging from about 3.5 mg to about 7 mg buprenorphine base or an equimolar amount of a pharmaceutically acceptable salt thereof and providing a size of said buprenorphine-containing matrix layer providing the area of release ranging from about 10 cm² to about 13 cm²; and
the third transdermal therapeutic system containing an amount of said buprenorphine ranging from about 6.5 mg to about 14 mg buprenorphine base or an equimolar amount of a pharmaceutically acceptable salt thereof and providing a size of said buprenorphine-containing matrix layer providing the area of release ranging from about 20 cm² to about 26 cm²; and
the fourth transdermal therapeutic system containing an amount of said buprenorphine ranging from about 11.5 mg to about 21 mg buprenorphine base or an equimolar amount of a pharmaceutically acceptable salt thereof and providing a size of said buprenorphine-containing matrix layer providing the area of release ranging from about 30 cm² to about 39 cm²; and
the fifth transdermal therapeutic system containing an amount of said buprenorphine ranging from about 15 mg to about 28 mg buprenorphine base or an equimolar amount of a pharmaceutically acceptable salt thereof and providing a size of said buprenorphine-containing matrix layer providing the area of release ranging from about 40 cm² to about 52 cm², wherein the five different transdermal therapeutic systems have increasing areas of release and increasing amounts of buprenorphine from the first to the fifth transdermal therapeutic system.

According to a certain embodiment of the invention, the set of different transdermal therapeutic systems comprises:

the first transdermal therapeutic system providing a size of said buprenorphine-containing matrix layer providing the area of release ranging from about 5 cm² to about 6 cm²;
the second transdermal therapeutic system providing a size of said buprenorphine-containing matrix layer providing the area of release ranging from about 10 cm² to about 12; and
the third transdermal therapeutic system providing a size of said buprenorphine-containing matrix layer providing the area of release ranging from about 20 cm² to about 24 cm²; and
the fourth transdermal therapeutic system providing a size of said buprenorphine-containing matrix layer providing the area of release ranging from about 30 cm² to about 36 cm²; and
the fifth transdermal therapeutic system providing a size of said buprenorphine-containing matrix layer providing the area of release ranging from about 40 cm² to about 48 cm², wherein the five different transdermal therapeutic systems have increasing areas of release from the first to the fifth transdermal therapeutic system.

According to a certain embodiment of the invention, the set of different transdermal therapeutic systems comprises:

the first transdermal therapeutic system containing an amount of said buprenorphine ranging from about 1 mg to about 3 mg buprenorphine base or an equimolar amount of a pharmaceutically acceptable salt thereof and providing a size of said buprenorphine-containing matrix layer providing the area of release ranging from about 5 cm² to about 6 cm²;
the second transdermal therapeutic system containing an amount of said buprenorphine ranging from about 3.5 mg to about 6 mg buprenorphine base or an equimolar amount of a pharmaceutically acceptable salt thereof and providing a size of said buprenorphine-containing matrix layer providing the area of release ranging from about 10 cm² to about 12 cm²; and
the third transdermal therapeutic system containing an amount of said buprenorphine ranging from about 6.5 mg to about 12 mg buprenorphine base or an equimolar amount of a pharmaceutically acceptable salt thereof and providing a size of said buprenorphine-containing matrix layer providing the area of release ranging from about 20 cm² to about 24 cm²; and
the fourth transdermal therapeutic system containing an amount of said buprenorphine ranging from about 12.5 mg to about 18 mg buprenorphine base or an equimolar amount of a pharmaceutically acceptable salt thereof and providing a size of said buprenorphine-containing matrix layer providing the area of release ranging from about 30 cm² to about 36 cm²; and
the fifth transdermal therapeutic system containing an amount of said buprenorphine ranging from about 18.5 mg to about 24 mg buprenorphine base or an equimolar amount of a pharmaceutically acceptable salt thereof and providing a size of said buprenorphine-containing matrix layer providing the area of release ranging from about 40 cm² to about 48 cm², wherein the five different transdermal therapeutic systems have increasing areas of release and increasing amounts of buprenorphine from the first to the fifth transdermal therapeutic system.

According to the invention, the set as described in the previous paragraphs provides from the first to the fifth transdermal therapeutic system increasing amounts of buprenorphine base or an equimolar amount of a pharmaceutically acceptable salt thereof and increasing sizes of said buprenorphine-containing matrix layer providing the area of release.

According to one aspect, the invention relates to a set as described in the previous paragraphs for use in a method of treating pain.

### Method of Treatment

According to the invention, the method of treating pain by applying the transdermal therapeutic system for the transdermal administration of buprenorphine as described above in detail comprises in particular the application of the TTS for about 7 days (corresponding to about 168 hours) on the skin of a patient referring to a once a week exchange mode or dosing regimen. According to other methods in accordance with the invention the TTS can be applied for more than 4 days corresponding to more than 96 hours, or about 5 days corresponding to about 120 hours and about 6 days corresponding to about 144 hours. The application for about 168 hours is preferred.

According to one aspect, the invention relates to a method of treating pain in a patient wherein said patient is treated with one appropriately selected TTS from a set of two (first and second, or second and third, or third and fourth, or fourth and fifth TTS, or any other combination of two of the five different dosage strengths), three (first to third, or second to fourth or third to fifth TTS, or any other combination of three of the five different dosage strengths), four (first to fourth or second to fifth TTS, or any other combination of four of the five different dosage strengths) or five (first to fifth TTS) different transdermal therapeutic systems corresponding to different dosage strengths and corresponding different nominal mean release rates and/or mean release rates over about 168 hours of administration, wherein:

the first transdermal therapeutic system contains an amount of said buprenorphine ranging from about 1 mg to about 4 mg buprenorphine base or an equimolar amount of a pharmaceutically acceptable salt thereof and provides a size of said buprenorphine-containing matrix layer providing the area of release ranging from more than 4.8 cm² to about 8 cm² and provides a mean release rate of buprenorphine of at least about 2 μg/hr, or of from about 2.5 to about 7.5 μg/hr or from about 4 to about 6 μg/hr, and/or provides a nominal mean release rate of buprenorphine of about 5 μg/hr over about 168 hours of administration; and
the second transdermal therapeutic system contains an amount of said buprenorphine ranging from about 3.5 mg to about 8 mg buprenorphine base or an equimolar amount of a pharmaceutically acceptable salt thereof and provides a size of said buprenorphine-containing matrix layer providing the area of release ranging from more than 9.5 cm² to about 15 cm² and provides a mean release rate of buprenorphine of at least about 6 μg/hr, or of from about 8 to about 12 μg/hr or from about 9 to about 11 μg/hr, and/or provides a nominal mean release rate of buprenorphine of about 10 μg/hr over about 168 hours of administration; and
the third transdermal therapeutic system contains an amount of said buprenorphine ranging from about 6.5 mg to about 16 mg buprenorphine base or an equimolar amount of a pharmaceutically acceptable salt thereof and provides a size of said buprenorphine-containing matrix layer providing the area of release ranging from more than 19 cm² to about 30 cm² and provides a mean release rate of buprenorphine of at least about 11 μg/hr, or of from about 15 to about 25 μg/hr or from about 17 to about 22 μg/hr, and/or provides a nominal mean release rate of buprenorphine of about 20 μg/hr over about 168 hours of administration; and
the fourth transdermal therapeutic system contains an amount of said buprenorphine ranging from about 11.5 mg to about 24 mg buprenorphine base or an equimolar amount of a pharmaceutically acceptable salt thereof and provides a size of said buprenorphine-containing matrix layer providing the area of release ranging from more than 28.5 cm² to about 45 cm² and provides a mean release rate of buprenorphine of at least about 21 μg/hr, or of from about 26 to about 35 μg/hr or from about 27 to about 32 μg/hr, and/or provides a nominal mean release rate of buprenorphine of about 30 μg/hr over about 168 hours of administration; and
the fifth transdermal therapeutic system contains an amount of said buprenorphine ranging from about 15 mg to about 32 mg buprenorphine base or an equimolar amount of a pharmaceutically acceptable salt thereof and provides a size of said buprenorphine-containing matrix layer providing the area of release ranging from more than 38 cm² to about 60 cm² and provides a mean release rate of buprenorphine of at least about 31 μg/hr, or of from about 36 to about 45 μg/hr or from about 38 to about 42 μg/hr, and/or provides a nominal mean release rate of buprenorphine of about 40 μg/hr over about 168 hours of administration.

The invention relates also to a method of treating pain in accordance with the previous paragraph wherein the set of five different transdermal therapeutic systems comprises

the first transdermal therapeutic system containing an amount of said buprenorphine ranging from about 1 mg to about 3.5 mg buprenorphine base or an equimolar amount of a pharmaceutically acceptable salt thereof and providing a size of said buprenorphine-containing matrix layer providing the area of release ranging from about 5 cm² to about 7 cm² and providing a mean release rate of buprenorphine of at least about 2 μg/hr, or of from about 2.5 to about 7.5 μg/hr or from about 4 to about 6 μg/hr, and/or providing a nominal mean release rate of buprenorphine of about 5 μg/hr over about 168 hours of administration; and
the second transdermal therapeutic system containing an amount of said buprenorphine ranging from about 3.5 mg to about 7 mg buprenorphine base or an equimolar amount of a pharmaceutically acceptable salt thereof and providing a size of said buprenorphine-containing matrix layer providing the area of release ranging from about 10 cm² to about 13 cm² and providing a mean release rate of buprenorphine of at least about 6 μg/hr, or of from about 8 to about 12 μg/hr or from about 9 to about 11 μg/hr, and/or providing a nominal mean release rate of buprenorphine of about 10 μg/hr over about 168 hours of administration; and
the third transdermal therapeutic system containing an amount of said buprenorphine ranging from about 6.5 mg to about 14 mg buprenorphine base or an equimolar amount of a pharmaceutically acceptable salt thereof and providing a size of said buprenorphine-containing matrix layer providing the area of release ranging from about 20 cm² to about 26 cm² and providing a mean release rate of buprenorphine of at least about 11 μg/hr, or of from about 15 to about 25 μg/hr or from about 17 to about 22 μg/hr, and/or providing a nominal mean release rate of buprenorphine of about 20 μg/hr over about 168 hours of administration; and
the fourth transdermal therapeutic system containing an amount of said buprenorphine ranging from about 11.5 mg to about 21 mg buprenorphine base or an equimolar amount of a pharmaceutically acceptable salt thereof and providing a size of said buprenorphine-containing matrix layer providing the area of release ranging from about 30 cm² to about 39 cm² and providing a mean release rate of buprenorphine of at least about 21 μg/hr, or of from about 26 to about 35 μg/hr or from about 27 to about 32 μg/hr, and/or providing a nominal mean release rate of buprenorphine of about 30 μg/hr over about 168 hours of administration; and
the fifth transdermal therapeutic system containing an amount of said buprenorphine ranging from about 15 mg to about 28 mg buprenorphine base or an equimolar amount of a pharmaceutically acceptable salt thereof and providing a size of said buprenorphine-containing matrix layer providing the area of release ranging from about 40 cm² to about 52 cm² and providing a mean release rate of buprenorphine of at least about 31 μg/hr, or of from about 36 to about 45 μg/hr or from about 38 to about 42 μg/hr, and/or providing a nominal mean release rate of buprenorphine of about 40 μg/hr over about 168 hours of administration.

The invention relates also to a method of treatment in accordance with the previous paragraphs wherein the set of five different transdermal therapeutic systems comprises

the first transdermal therapeutic system containing an amount of said buprenorphine ranging from about about 1 mg to about 3 mg buprenorphine base or an equimolar amount of a pharmaceutically acceptable salt thereof and providing a size of said buprenorphine-containing matrix layer providing the area of release ranging from about 5 cm² to about 6 cm² and providing a mean release rate of buprenorphine of at least about 2 μg/hr, or of from about 2.5 to about 7.5 μg/hr or from about 4 to about 6 μg/hr, and/or providing a nominal mean release rate of buprenorphine of about 5 μg/hr over about 168 hours of administration;
the second transdermal therapeutic system containing an amount of said buprenorphine ranging from about 3.5 mg to about 6 mg buprenorphine base or an equimolar amount of a pharmaceutically acceptable salt thereof and providing a size of said buprenorphine-containing matrix layer providing the area of release ranging from about 10 cm² to about 12 cm² and providing a mean release rate of buprenorphine of at least about 6 μg/hr, or of from about 8 to about 12 μg/hr or from about 9 to about 11 μg/hr, and/or providing a nominal mean release rate of buprenorphine of about 10 μg/hr over about 168 hours of administration; and
the third transdermal therapeutic system containing an amount of said buprenorphine ranging from about 6.5 mg to about 12 mg buprenorphine base or an equimolar amount of a pharmaceutically acceptable salt thereof and providing a size of said buprenorphine-containing matrix layer providing the area of release ranging from about 20 cm² to about 24 cm² and providing a mean release rate of buprenorphine of at least about 11 μg/hr, or of from about 15 to about 25 μg/hr or from about 17 to about 22 μg/hr, and/or providing a nominal mean release rate of buprenorphine of about 20 μg/hr over about 168 hours of administration; and
the fourth transdermal therapeutic system containing an amount of said buprenorphine ranging from about 12.5 mg to about 18 mg buprenorphine base or an equimolar amount of a pharmaceutically acceptable salt thereof and providing a size of said buprenorphine-containing matrix layer providing the area of release ranging from about 30 cm² to about 36 cm² and providing a mean release rate of buprenorphine of at least about 21 μg/hr, or of from about 26 to about 35 μg/hr or from about 27 to about 32 μg/hr, and/or providing a nominal mean release rate of buprenorphine of about 30 μg/hr over about 168 hours of administration; and
the fifth transdermal therapeutic system containing an amount of said buprenorphine ranging from about 18.5 mg to about 24 mg buprenorphine base or an equimolar amount of a pharmaceutically acceptable salt thereof and providing a size of said buprenorphine-containing matrix layer providing the area of release ranging from about 40 cm² to about 48 cm² and providing a mean release rate of buprenorphine of at least about 31 μg/hr, or of from about 36 to about 45 μg/hr or from about 38 to about 42 μg/hr, and/or providing a nominal mean release rate of buprenorphine of about 40 μg/hr over about 168 hours of administration.

The invention relates also to a method of treating pain in a patient by applying a transdermal therapeutic system comprising buprenorphine for the transdermal administration of buprenorphine for 7 days on the skin of a patient, wherein the transdermal therapeutic system is selected from:

a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a nominal mean release rate of about 5 μg/hr and/or providing a mean AUCt of more than 7,000 pg·hr/ml, preferably more than 8,000 pg·hr/ml, or of from more than 7,000 pg·hr/ml to about 16,000 pg·hr/ml, or of from more than 8,000 pg·hr/ml to about 16,000 pg·hr/ml over about 168 hours of administration after a single-dose administration to a subject population; and
a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a nominal mean release rate of about 10 μg/hr and/or providing a mean AUCt of more than 14,000 pg·hr/ml, preferably of more than 16,000 pg·hr/ml, or of from more than 14,000 pg·hr/ml to about 32,000 pg·hr/ml, or of from more than 16,000 pg·hr/ml to about 32,000 pg·hr/ml over about 168 hours of administration after a single-dose administration to a subject population; and
a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a nominal mean release rate of about 20 μg/hr and/or providing a mean AUCt of more than 28,000 pg·hr/ml, preferably of more than 32,000 pg·hr/ml, or of from more than 28,000 pg·hr/ml to about 64,000 pg·hr/ml, or of from more than 32,000 pg·hr/ml to about 64,000 pg·hr/ml over about 168 hours of administration after a single-dose administration to a subject population; and
a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a nominal mean release rate of about 30 μg/hr and/or providing a mean AUCt of more than 42,000 pg·hr/ml, preferably of more than 48,000 pg·hr/ml, or of from more than 42,000 pg·hr/ml to about 96,000 pg·hr/ml, or of from more than 48,000 pg·hr/ml to about 96,000 pg·hr/ml over about 168 hours of administration after a single-dose administration to a subject population; and
a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a nominal mean release rate of about 40 μg/hr and/or providing a mean AUCt of more than 62,000 pg·hr/ml, preferably of more than 64,000 pg·hr/ml, or of from more than 62,000 pg·hr/ml to about 128,000 pg·hr/ml, or of from more than 64,000 pg·hr/ml to about 128,000 pg·hr/ml over about 168 hours of administration after a single-dose administration to a subject population.

The invention relates also to a method of treating pain in a patient by applying a transdermal therapeutic system comprising buprenorphine for the transdermal administration of buprenorphine for 7 days on the skin of a patient, wherein said transdermal therapeutic system is selected from:

a first transdermal therapeutic system containing an amount of said buprenorphine ranging from about 1 mg to about 3.5 mg buprenorphine base or an equimolar amount of a pharmaceutically acceptable salt thereof and providing a size of the area of release ranging from about 5 cm² to about 7 cm² and providing a nominal mean release rate of about 5 μg/hr and/or providing a mean AUCt of more than 7,000 pg·hr/ml, preferably more than 8,000 pg·hr/ml, or of from more than 7,000 pg·hr/ml to about 16,000 pg·hr/ml, or of from more than 8,000 pg·hr/ml to about 16,000 pg·hr/ml over about 168 hours of administration after a single-dose administration to a subject population; and
a second transdermal therapeutic system containing an amount of said buprenorphine ranging from about 3.5 mg to about 7 mg buprenorphine base or an equimolar amount of a pharmaceutically acceptable salt thereof and providing a size of the area of release ranging from about 10 cm² to about 13 cm² and providing a nominal mean release rate of about 10 μg/hr and/or providing a mean AUCt of more than 14,000 pg·hr/ml, preferably of more than 16,000 pg·hr/ml, or of from more than 14,000 pg·hr/ml to about 32,000 pg·hr/ml, or of from more than 16,000 pg·hr/ml to about 32,000 pg·hr/ml over about 168 hours of administration after a single-dose administration to a subject population; and
a third transdermal therapeutic system containing an amount of said buprenorphine ranging from about 6.5 mg to about 14 mg buprenorphine base or an equimolar amount of a pharmaceutically acceptable salt thereof and providing a size of the area of release ranging from about 20 cm² to about 26 cm² and providing a nominal mean release rate of about 20 μg/hr and/or providing a mean AUCt of more than 28,000 pg·hr/ml, preferably of more than 32,000 pg·hr/ml, or of from more than 28,000 pg·hr/ml to about 64,000 pg·hr/ml, or of from more than 32,000 pg·hr/ml to about 64,000 pg·hr/ml over about 168 hours of administration after a single-dose administration to a subject population; and
a fourth transdermal therapeutic system containing an amount of said buprenorphine ranging from about 11.5 mg to about 21 mg buprenorphine base or an equimolar amount of a pharmaceutically acceptable salt thereof and providing a size of the area of release ranging from about 30 cm² to about 39 cm² and providing a nominal mean release rate of about 30 μg/hr and/or providing a mean AUCt of more than 42,000 pg·hr/ml, preferably of more than 48,000 pg·hr/ml, or of from more than 42,000 pg·hr/ml to about 96,000 pg·hr/ml, or of from more than 48,000 pg·hr/ml to about 96,000 pg·hr/ml over about 168 hours of administration after a single-dose administration to a subject population; and
a fifth transdermal therapeutic system containing an amount of said buprenorphine ranging from about 15 mg to about 28 mg buprenorphine base or an equimolar amount of a pharmaceutically acceptable salt thereof and providing a size of the area of release ranging from about 40 cm² to about 52 cm² and providing a nominal mean release rate of about 40 μg/hr and/or providing a mean AUCt of more than 62,000 pg·hr/ml, preferably of more than 64,000 pg·hr/ml, or of from more than 62,000 pg·hr/ml to about 128,000 pg·hr/ml, or of from more than 64,000 pg·hr/ml to about 128,000 pg·hr/ml over about 168 hours of administration after a single-dose administration to a subject population.

The invention relates also to a method of treating pain in a patient by applying a transdermal therapeutic system comprising buprenorphine for the transdermal administration of buprenorphine for 7 days on the skin of a patient, wherein the said transdermal therapeutic system is selected from:

a first transdermal therapeutic system containing an amount of said buprenorphine ranging from about 1 mg to about 3 mg buprenorphine base or an equimolar amount of a pharmaceutically acceptable salt thereof and providing a size of the area of release ranging from about 5 cm² to about 6 cm² and providing a nominal mean release rate of about 5 μg/hr and/or providing a mean AUCt of more than 7,000 pg·hr/ml, preferably more than 8,000 pg·hr/ml, or of from more than 7,000 pg·hr/ml to about 16,000 pg·hr/ml, or of from more than 8,000 pg·hr/ml to about 16,000 pg·hr/ml over about 168 hours of administration after a single-dose administration to a subject population; and
a second transdermal therapeutic system containing an amount of said buprenorphine ranging from about 3.5 mg to about 6 mg buprenorphine base or an equimolar amount of a pharmaceutically acceptable salt thereof and providing a size of the area of release ranging from about 10 cm² to about 12 cm² and providing a nominal mean release rate of about 10 μg/hr and/or providing a mean AUCt of more than 14,000 pg·hr/ml, preferably of more than 16,000 pg·hr/ml, or of from more than 14,000 pg·hr/ml to about 32,000 pg·hr/ml, or of from more than 16,000 pg·hr/ml to about 32,000 pg·hr/ml over about 168 hours of administration after a single-dose administration to a subject population; and
a third transdermal therapeutic system containing an amount of said buprenorphine ranging from about 6.5 mg to about 12 mg buprenorphine base or an equimolar amount of a pharmaceutically acceptable salt thereof and providing a size of the area of release ranging from about 20 cm² to about 24 cm² and providing a nominal mean release rate of about 20 μg/hr and/or providing a mean AUCt of more than 28,000 pg·hr/ml, preferably of more than 32,000 pg·hr/ml, or of from more than 28,000 pg·hr/ml to about 64,000 pg·hr/ml, or of from more than 32,000 pg·hr/ml to about 64,000 pg·hr/ml over about 168 hours of administration after a single-dose administration to a subject population; and
a fourth transdermal therapeutic system containing an amount of said buprenorphine ranging from about 12.5 mg to about 18 mg buprenorphine base or an equimolar amount of a pharmaceutically acceptable salt thereof and providing a size of the area of release ranging from about 30 cm² to about 36 cm² and providing a nominal mean release rate of about 30 μg/hr and/or providing a mean AUCt of more than 42,000 pg·hr/ml, preferably of more than 48,000 pg·hr/ml, or of from more than 42,000 pg·hr/ml to about 96,000 pg·hr/ml, or of from more than 48,000 pg·hr/ml to about 96,000 pg·hr/ml over about 168 hours of administration after a single-dose administration to a subject population; and
a fifth transdermal therapeutic system containing an amount of said buprenorphine ranging from about 18.5 mg to about 24 mg buprenorphine base or an equimolar amount of a pharmaceutically acceptable salt thereof and providing a size of the area of release ranging from about 40 cm² to about 48 cm² and providing a nominal mean release rate of about 40 μg/hr and/or providing a mean AUCt of more than 62,000 pg·hr/ml, preferably of more than 64,000 pg·hr/ml, or of from more than 62,000 pg·hr/ml to about 128,000 pg·hr/ml, or of from more than 64,000 pg·hr/ml to about 128,000 pg·hr/ml over about 168 hours of administration after a single-dose administration to a subject population.

According to one aspect, the invention relates to a method of treatment as described in the previous paragraphs, wherein the transdermal therapeutic system provides an arithmetic mean tmax from about 72 hr to about 132 hr, preferably from about 78 hr to about 126 hr, or from about 84 hr to about 120 hr after a single dose administration to a subject population.

### Medical Use

According to the invention, the transdermal therapeutic system as described above in detail is for use in a method of treating pain comprising in particular the application of the TTS for about 7 days (corresponding to about 168 hours) on the skin of a patient which refers to a once a week exchange mode or dosing regimen. According to other methods in accordance with the invention the TTS can be applied for more than 4 days corresponding to more than 96 hours, or about 5 days corresponding to about 120 hours and about 6 days corresponding to about 144 hours. The application for about 168 hours is preferred.

According to one aspect, the invention relates to a transdermal therapeutic system for use in a method of treating pain in a patient wherein said patient is treated with one appropriately selected TTS from a set of two (first and second, or second and third, or third and fourth, or fourth and fifth TTS, or any other combination of two of the five different dosage strengths), three (first to third, or second to fourth or third to fifth TTS), four (first to fourth or second to fifth TTS) or five (first to fifth TTS) different transdermal therapeutic systems corresponding to different dosage strengths and corresponding different nominal mean relase rates and/or mean release rates over about 168 hours of administration, wherein: the first transdermal therapeutic system contains an amount of said buprenorphine ranging from about 1 mg to about 4 mg buprenorphine base or an equimolar amount of a pharmaceutically acceptable salt thereof and provides a size of said buprenorphine-containing matrix layer providing the area of release ranging from more than 4.8 cm² to about 8 cm² and provides a mean release rate of buprenorphine of at least about 2 μg/hr, or of from about 2.5 to about 7.5 μg/hr or from about 4 to about 6 μg/hr, and/or provides a nominal mean release rate of buprenorphine of about 5 μg/hr over about 168 hours of administration; and

the second transdermal therapeutic system contains an amount of said buprenorphine ranging from about 3.5 mg to about 8 mg buprenorphine base or an equimolar amount of a pharmaceutically acceptable salt thereof and provides a size of said buprenorphine-containing matrix layer providing the area of release ranging from more than 9.5 cm² to about 15 cm² and provides a mean release rate of buprenorphine of at least about 6 μg/hr, or of from about 8 to about 12 μg/hr or from about 9 to about 11 μg/hr, and/or provides a nominal mean release rate of buprenorphine of about 10 μg/hr over about 168 hours of administration; and
the third transdermal therapeutic system contains an amount of said buprenorphine ranging from about 6.5 mg to about 16 mg buprenorphine base or an equimolar amount of a pharmaceutically acceptable salt thereof and provides a size of said buprenorphine-containing matrix layer providing the area of release ranging from more than 19 cm² to about 30 cm² and provides a mean release rate of buprenorphine of buprenorphine of at least about 11 μg/hr, or of from about 15 to about 25 μg/hr or from about 17 to about 22 μg/hr, and/or provides a nominal mean release rate of about 20 μg/hr over about 168 hours of administration; and
the fourth transdermal therapeutic system contains an amount of said buprenorphine ranging from about 11.5 mg to about 24 mg buprenorphine base or an equimolar amount of a pharmaceutically acceptable salt thereof and provides a size of said buprenorphine-containing matrix layer providing the area of release ranging from more than 28.5 cm² to about 45 cm² and provides a mean release rate of buprenorphine of at least about 21 μg/hr, or of from about 26 to about 35 μg/hr or from about 27 to about 32 μg/hr, and/or provides a nominal mean release rate of buprenorphine of about 30 μg/hr over about 168 hours of administration; and
the fifth transdermal therapeutic system contains an amount of said buprenorphine ranging from about 15 mg to about 32 mg buprenorphine base or an equimolar amount of a pharmaceutically acceptable salt thereof and provides a size of said buprenorphine-containing matrix layer providing the area of release ranging from more than 38 cm² to about 60 cm² and provides a mean release rate of buprenorphine of at least about 31 μg/hr, or of from about 36 to about 45 μg/hr or from about 38 to about 42 μg/hr, and/or provides a nominal mean release rate of buprenorphine of about 40 μg/hr over about 168 hours of administration.

The invention relates also to a transdermal therapeutic system for use in a method of treating pain in accordance with the previous paragraph, wherein: the first transdermal therapeutic system contains an amount of said buprenorphine ranging from about 1 mg to about 3.5 mg buprenorphine base or an equimolar amount of a pharmaceutically acceptable salt thereof and provides a size of said buprenorphine-containing matrix layer providing the area of release ranging from about 5 cm² to about 7 cm² and provides a mean release rate of buprenorphine of at least about 2 μg/hr, or of from about 2.5 to about 7.5 μg/hr or from about 4 to about 6 μg/hr, and/or provides a nominal mean release rate of buprenorphine of about 5 μg/hr over about 168 hours of administration; and

the second transdermal therapeutic system contains an amount of said buprenorphine ranging from about 3.5 mg to about 7 mg buprenorphine base or an equimolar amount of a pharmaceutically acceptable salt thereof and provides a size of said buprenorphine-containing matrix layer providing the area of release ranging from about 10 cm² to about 13 cm² and provides a mean release rate of buprenorphine of at least about 6 μg/hr, or of from about 8 to about 12 μg/hr or from about 9 to about 11 μg/hr, and/or provides a nominal mean release rate of buprenorphine of about 10 μg/hr over about 168 hours of administration; and
the third transdermal therapeutic system contains an amount of said buprenorphine ranging from about 6.5 mg to about 14 mg buprenorphine base or an equimolar amount of a pharmaceutically acceptable salt thereof and provides a size of said buprenorphine-containing matrix layer providing the area of release ranging from about 20 cm² to about 26 cm² and provides a mean release rate of buprenorphine of buprenorphine of at least about 11 μg/hr, or of from about 15 to about 25 μg/hr or from about 17 to about 22 μg/hr, and/or provides a nominal mean release rate of buprenorphine of about 20 μg/hr over about 168 hours of administration; and
the fourth transdermal therapeutic system contains an amount of said buprenorphine ranging from about 11.5 mg to about 21 mg buprenorphine base or an equimolar amount of a pharmaceutically acceptable salt thereof and provides a size of said buprenorphine-containing matrix layer providing the area of release ranging from about 30 cm² to about 39 cm² and provides a mean release rate of buprenorphine of at least about 21 μg/hr, or of from about 26 to about 35 μg/hr or from about 27 to about 32 μg/hr, and/or provides a nominal mean release rate of buprenorphine of about 30 μg/hr over about 168 hours of administration; and
the fifth transdermal therapeutic system contains an amount of said buprenorphine ranging from about 15 mg to about 28 mg buprenorphine base or an equimolar amount of a pharmaceutically acceptable salt thereof and provides a size of said buprenorphine-containing matrix layer providing the area of release ranging from about 40 cm² to about 52 cm² and provides a mean release rate of buprenorphine of at least about 31 μg/hr, or of from about 36 to about 45 μg/hr or from about 38 to about 42 μg/hr, and/or provides a nominal mean release rate of buprenorphine of about 40 μg/hr over about 168 hours of administration.

The invention relates also to a transdermal therapeutic system for use in a method of treating pain in accordance with the previous paragraphs, wherein: the first transdermal therapeutic system contains an amount of said buprenorphine ranging from about about 1 mg to about 3 mg buprenorphine base or an equimolar amount of a pharmaceutically acceptable salt thereof and provides a size of said buprenorphine-containing matrix layer providing the area of release ranging from about 5 cm² to about 6 cm² and provides a mean release rate of buprenorphine of at least about 2 μg/hr, or of from about 2.5 to about 7.5 μg/hr or from about 4 to about 6 μg/hr, and/or provides a nominal mean release rate of buprenorphine of about 5 μg/hr over about 168 hours of administration;
the second transdermal therapeutic system contains an amount of said buprenorphine ranging from about 3.5 mg to about 6 mg buprenorphine base or an equimolar amount of a pharmaceutically acceptable salt thereof and provides a size of said buprenorphine-containing matrix layer providing the area of release ranging from about 10 cm² to about 12 cm² and provides a mean release rate of buprenorphine of at least about 6 μg/hr, or of from about 8 to about 12 μg/hr or from about 9 to about 11 μg/hr, and/or provides a nominal mean release rate of buprenorphine of about 10 μg/hr over about 168 hours of administration; and
   the third transdermal therapeutic system contains an amount of said buprenorphine ranging from about 6.5 mg to about 12 mg buprenorphine base or an equimolar amount of a pharmaceutically acceptable salt thereof and provides a size of said buprenorphine-containing matrix layer providing the area of release ranging from about 20 cm² to about 24 cm² and provides a mean release rate of buprenorphine of buprenorphine of at least about 11 μg/hr, or of from about 15 to about 25 μg/hr or from about 17 to about 22 μg/hr, and/or provides a nominal mean release rate of buprenorphine of about 20 μg/hr over about 168 hours of administration; and
   the fourth transdermal therapeutic system contains an amount of said buprenorphine ranging from about 12.5 mg to about 18 mg buprenorphine base or an equimolar amount of a pharmaceutically acceptable salt thereof and provides a size of said buprenorphine-containing matrix layer providing the area of release ranging from about 30 cm² to about 36 cm² and provides a mean release rate of buprenorphine of at least about 21 μg/hr, or of from about 26 to about 35 μg/hr or from about 27 to about 32 μg/hr, and/or provides a nominal mean release rate of buprenorphine of about 30 μg/hr over about 168 hours of administration; and
   the fifth transdermal therapeutic system contains an amount of said buprenorphine ranging from about 18.5 mg to about 24 mg buprenorphine base or an equimolar amount of a pharmaceutically acceptable salt thereof and provides a size of said buprenorphine-containing matrix layer providing the area of release ranging from about 40 cm² to about 48 cm² and provides a mean release rate of buprenorphine of at least about 31 μg/hr, or of from about 36 to about 45 μg/hr or from about 38 to about 42 μg/hr, and/or provides a nominal mean release rate of buprenorphine of about 40 μg/hr over about 168 hours of administration.

The invention relates also to a transdermal therapeutic system for use in a method of treating pain in a patient by applying one appropriately selected transdermal therapeutic system comprising buprenorphine on the skin of said patient for 7 days, wherein said TTS is selected from:

a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a nominal mean release rate of about 5 μg/hr and/or providing a mean AUCt of more than 7,000 pg·hr/ml, preferably more than 8,000 pg·hr/ml, or of from more than 7,000 pg·hr/ml to about 16,000 pg·hr/ml, or of from more than 8,000 pg·hr/ml to about 16,000 pg·hr/ml over about 168 hours of administration after a single-dose administration to a subject population; and
a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a nominal mean release rate of about 10 μg/hr and/or providing a mean AUCt of more than 14,000 pg·hr/ml, preferably of more than 16,000 pg·hr/ml, or of from more than 14,000 pg·hr/ml to about 32,000 pg·hr/ml, or of from more than 16,000 pg·hr/ml to about 32,000 pg·hr/ml over about 168 hours of administration after a single-dose administration to a subject population; and
a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a nominal mean release rate of about 20 μg/hr and/or providing a mean AUCt of more than 28,000 pg·hr/ml, preferably of more than 32,000 pg·hr/ml, or of from more than 28,000 pg·hr/ml to about 64,000 pg·hr/ml, or of from more than 32,000 pg·hr/ml to about 64,000 pg·hr/ml over about 168 hours of administration after a single-dose administration to a subject population; and
a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a nominal mean release rate of about 30 μg/hr and/or providing a mean AUCt of more than 42,000 pg·hr/ml, preferably of more than 48,000 pg·hr/ml, or of from more than 42,000 pg·hr/ml to about 96,000 pg·hr/ml, or of from more than 48,000 pg·hr/ml to about 96,000 pg·hr/ml over about 168 hours of administration after a single-dose administration to a subject population; and
a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a nominal mean release rate of about 40 μg/hr and/or providing a mean AUCt of more than 62,000 pg·hr/ml, preferably of more than 64,000 pg·hr/ml, or of from more than 62,000 pg·hr/ml to about 128,000 pg·hr/ml, or of from more than 64,000 pg·hr/ml to about 128,000 pg·hr/ml over about 168 hours of administration after a single-dose administration to a subject population.

The invention relates also to a transdermal therapeutic system for use in a method of treating pain in a patient by applying one appropriately selected transdermal therapeutic system comprising buprenorphine on the skin of said patient for 7 days, wherein said TTS is selected from:

a first transdermal therapeutic system containing an amount of said buprenorphine ranging from about 1 mg to about 3.5 mg buprenorphine base or an equimolar amount of a pharmaceutically acceptable salt thereof and providing a size of the area of release ranging from about 5 cm² to about 7 cm² and providing a nominal mean release rate of about 5 μg/hr and/or providing a mean AUCt of more than 7,000 pg·hr/ml, preferably more than 8,000 pg·hr/ml, or of from more than 7,000 pg·hr/ml to about 16,000 pg·hr/ml, or of from more than 8,000 pg·hr/ml to about 16,000 pg·hr/ml over about 168 hours of administration after a single-dose administration to a subject population; and
a second transdermal therapeutic system containing an amount of said buprenorphine ranging from about 3.5 mg to about 7 mg buprenorphine base or an equimolar amount of a pharmaceutically acceptable salt thereof and providing a size of the area of release ranging from about 10 cm² to about 13 cm² and providing a nominal mean release rate of about 10 μg/hr and/or providing a mean AUCt of more than 14,000 pg·hr/ml, preferably of more than 16,000 pg·hr/ml, or of from more than 14,000 pg·hr/ml to about 32,000 pg·hr/ml, or of from more than 16,000 pg·hr/ml to about 32,000 pg·hr/ml over about 168 hours of administration after a single-dose administration to a subject population; and
a third transdermal therapeutic system containing an amount of said buprenorphine ranging from about 6.5 mg to about 14 mg buprenorphine base or an equimolar amount of a pharmaceutically acceptable salt thereof and providing a size of the area of release ranging from about 20 cm² to about 26 cm² and providing a nominal mean release rate of about 20 μg/hr and/or providing a mean AUCt of more than 28,000 pg·hr/ml, preferably of more than 32,000 pg·hr/ml, or of from more than 28,000 pg·hr/ml to about 64,000 pg·hr/ml, or of from more than 32,000 pg·hr/ml to about 64,000 pg·hr/ml over about 168 hours of administration after a single-dose administration to a subject population; and
a fourth transdermal therapeutic system containing an amount of said buprenorphine ranging from about 11.5 mg to about 21 mg buprenorphine base or an equimolar amount of a pharmaceutically acceptable salt thereof and providing a size of the area of release ranging from about 30 cm² to about 39 cm² and providing a nominal mean release rate of about 30 μg/hr and/or providing a mean AUCt of more than 42,000 pg·hr/ml, preferably of more than 48,000 pg·hr/ml, or of from more than 42,000 pg·hr/ml to about 96,000 pg·hr/ml, or of from more than 48,000 pg·hr/ml to about 96,000 pg·hr/ml over about 168 hours of administration after a single-dose administration to a subject population; and
a fifth transdermal therapeutic system containing an amount of said buprenorphine ranging from about 15 mg to about 28 mg buprenorphine base or an equimolar amount of a pharmaceutically acceptable salt thereof and providing a size of the area of release ranging from about 40 cm² to about 52 cm² and providing a nominal mean release rate of about 40 μg/hr and/or providing a mean AUCt of more than 62,000 pg·hr/ml, preferably of more than 64,000 pg·hr/ml, or of from more than 62,000 pg·hr/ml to about 128,000 pg·hr/ml, or of from more than 64,000 pg·hr/ml to about 128,000 pg·hr/ml over about 168 hours of administration after a single-dose administration to a subject population for use in a method of treating pain in a patient by applying one of said transdermal therapeutic systems for 7 days on the skin of a patient.

The invention relates also to a transdermal therapeutic system for use in a method of treating pain in a patient by applying one appropriately selected transdermal therapeutic system comprising buprenorphine on the skin of said patient for 7 days, wherein said TTS is selected from:

a first transdermal therapeutic system containing an amount of said buprenorphine ranging from about 1 mg to about 3 mg buprenorphine base or an equimolar amount of a pharmaceutically acceptable salt thereof and providing a size of the area of release ranging from about 5 cm² to about 6 cm² and providing a nominal mean release rate of about 5 μg/hr and/or providing a mean AUCt of more than 7,000 pg·hr/ml, preferably more than 8,000 pg·hr/ml, or of from more than 7,000 pg·hr/ml to about 16,000 pg·hr/ml, or of from more than 8,000 pg·hr/ml to about 16,000 pg·hr/ml over about 168 hours of administration after a single-dose administration to a subject population; and
a second transdermal therapeutic system containing an amount of said buprenorphine ranging from about 3.5 mg to about 6 mg buprenorphine base or an equimolar amount of a pharmaceutically acceptable salt thereof and providing a size of the area of release ranging from about 10 cm² to about 12 cm² and providing a nominal mean release rate of about 10 μg/hr and/or providing a mean AUCt of more than 14,000 pg·hr/ml, preferably of more than 16,000 pg·hr/ml, or of from more than 14,000 pg·hr/ml to about 32,000 pg·hr/ml, or of from more than 16,000 pg·hr/ml to about 32,000 pg·hr/ml over about 168 hours of administration after a single-dose administration to a subject population; and
a third transdermal therapeutic system containing an amount of said buprenorphine ranging from about 6.5 mg to about 12 mg buprenorphine base or an equimolar amount of a pharmaceutically acceptable salt thereof and providing a size of the area of release ranging from about 20 cm² to about 24 cm² and providing a nominal mean release rate of about 20 μg/hr and/or providing a mean AUCt of more than 28,000 pg·hr/ml, preferably of more than 32,000 pg·hr/ml, or of from more than 28,000 pg·hr/ml to about 64,000 pg·hr/ml, or of from more than 32,000 pg·hr/ml to about 64,000 pg·hr/ml over about 168 hours of administration after a single-dose administration to a subject population; and
a fourth transdermal therapeutic system containing an amount of said buprenorphine ranging from about 12.5 mg to about 18 mg buprenorphine base or an equimolar amount of a pharmaceutically acceptable salt thereof and providing a size of the area of release ranging from about 30 cm² to about 36 cm² and providing a nominal mean release rate of about 30 μg/hr and/or providing a mean AUCt of more than 42,000 pg·hr/ml, preferably of more than 48,000 pg·hr/ml, or of from more than 42,000 pg·hr/ml to about 96,000 pg·hr/ml, or of from more than 48,000 pg·hr/ml to about 96,000 pg·hr/ml over about 168 hours of administration after a single-dose administration to a subject population; and
a fifth transdermal therapeutic system containing an amount of said buprenorphine ranging from about 18.5 mg to about 24 mg buprenorphine base or an equimolar amount of a pharmaceutically acceptable salt thereof and providing a size of the area of release ranging from about 40 cm² to about 48 cm² and providing a nominal mean release rate of about 40 μg/hr and/or providing a mean AUCt of more than 62,000 pg·hr/ml, preferably of more than 64,000 pg·hr/ml, or of from more than 62,000 pg·hr/ml to about 128,000 pg·hr/ml, or of from more than 64,000 pg·hr/ml to about 128,000 pg·hr/ml over about 168 hours of administration after a single-dose administration to a subject population for use in a method of treating pain in a patient by applying one of said transdermal therapeutic systems for 7 days on the skin of a patient.

The invention relates also to a transdermal therapeutic system for use in a method of treating pain in accordance with the previous paragraphs wherein the transdermal therapeutic system provides an arithmethic mean tmax of from about 72 hr to about 132 hr, preferably of about 48 hr to about 132 hr, or more preferably of about 60 hr to about 120 hr after a single dose administration to a subject population.

### Release Characteristic

In accordance with the invention, the TTS is further characterized by the skin permeation rate determined by in vitro experiments carried out with the Franz diffusion cell (e.g., a 9 ml Franz diffusion cell), using human split thickness skin. Skin from cosmetic surgeries (female breast, date of birth 1989) can be used. A dermatome is used to prepare skin to a thickness of 800 μm, with an intact epidermis, in accordance with the OECD Guideline (adopted Apr. 13, 2004). Due to the prolonged test (168 hours) 800 μm skin is used instead of the recommended 200 to 400 μm skin. The receptor medium used is a phosphate buffer solution pH 5.5 with 0.1% saline azide as antibacteriological agent is used at a temperature of 32±1° C. Example formulations with an area of 1.163 cm² are punched from laminates, and in the present examples are each tested against 1.163 cm² samples of the commercial product Norspan®. The concentrations of buprenorphine in the acceptor medium of the Franz cell are measured.

The TTS according to the invention provides a mean cumulative skin permeation rate of more than 1.1 μg/cm²-hr, or more than 1.2 μg/cm²-hr, or more than 1.3 μg/cm²-hr over a 168 hours test, or of more than 1.4 μg/cm²-hr over a 168 hours test, or of 1.5 μg/cm²-hr or more over a 168 hours test, or from about 1.2 μg/cm²-hr to about 4 μg/cm²-hr, or from about 1.3 μg/cm²-hr to about 4 μg/cm²-hr, or from about 1.4 μg/cm²-hr to about 4 μg/cm²-hr, or from about 1.5 μg/cm²-hr to about 2 μg/cm²-hr over a 168 hours test. The commercial product Norspan® provides a mean cumulative skin permeation rate of about 1 μg/cm²-hr over a 168 hours test in said test.

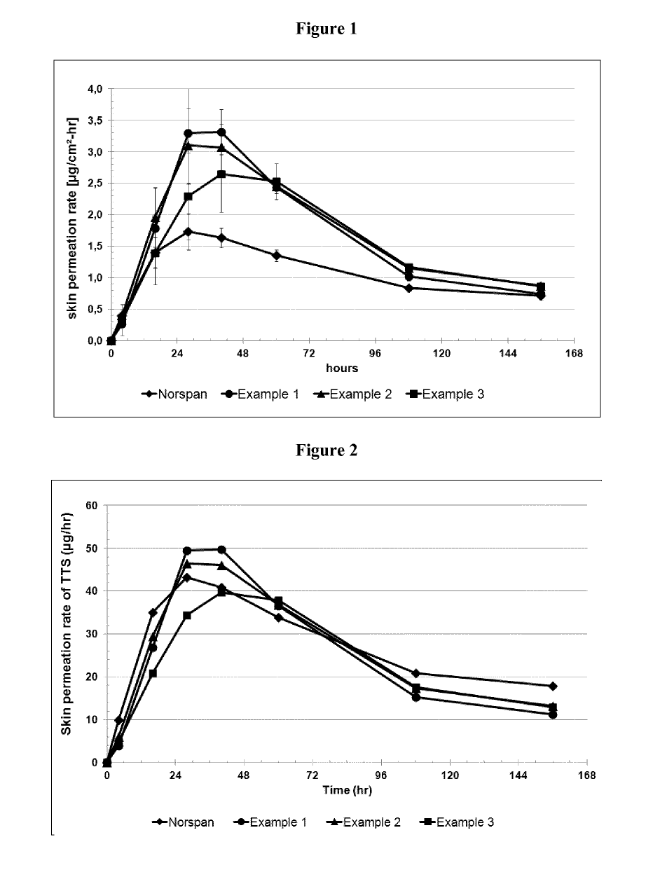

According to certain embodiments, the TTS provides a cumulative release as measured in a Franz diffusion cell as mentioned above of more than 185 μg/cm², or more than 200 μg/cm², or more than 220 μg/cm² over a time period of 168 hours, or of more than 235 μg/cm², or more than 250 μg/cm² over a time period of 168 hours, or from about 200 μg/cm² to about 400 μg/cm² over a time period of 168 hours, or from about 220 μg/cm² to about 350 μg/cm², or from about 235 μg/cm² to about 300 μg/cm², or from about 250 μg/cm² to about 300 μg/cm² over a time period of 168 hours. The commercial product Norspan® provides a cumulative release of about 175 μg/cm² in said test. As can be seen from FIG. 2, comparable skin permeation rates are measured using the 25 cm² Norspan® TTS including 20 mg buprenorphine base and TTS examples 1 to 3 in accordance with the invention with an area of release of 15 cm² and including 6.75 mg buprenorphine base. This corresponds to about a 40% size reduction and a reduction of about 66% in the amount of used buprenorphine base.

According to certain embodiments, the TTS provides a non-cumulative skin permeation rate of buprenorphine base as measured in a Franz diffusion cell of

1 μg/cm² to 10 μg/cm² in the first 8 hours,
10 μg/cm² to 60 μg/cm² from hour 8 to hour 24,
10 μg/cm² to 60 μg/cm² from hour 24 to hour 32,
30 μg/cm² to 100 μg/cm² from hour 32 to hour 48,
40 μg/cm² to 120 μg/cm² from hour 48 to hour 72,
50 μg/cm² to 150 μg/cm² from hour 72 to hour 144, and
10 μg/cm² to 50 μg/cm² from hour 144 to hour 168.

According to certain embodiments, the TTS provides a non-cumulative skin permeation rate of buprenorphine base as measured in a Franz diffusion cell of

1 μg/cm² to 6 μg/cm in the first 8 hours,
15 μg/cm² to 50 μg/cm² from hour 8 to hour 24,
15 μg/cm² to 50 μg/cm² from hour 24 to hour 32,
40 μg/cm² to 80 μg/cm² from hour 32 to hour 48,
50 μg/cm² to 100 μg/cm² from hour 48 to hour 72,
60 μg/cm² to 120 μg/cm² from hour 72 to hour 144, and
15 μg/cm² to 40 μg/cm² from hour 144 to hour 168.

According to certain embodiments, the TTS provides a non-cumulative skin permeation rate of buprenorphine base as measured in a Franz diffusion cell of

1 μg/cm² to 4 μg/cm in the first 8 hours,
20 μg/cm² to 40 μg/cm² from hour 8 to hour 24,
20 μg/cm² to 40 μg/cm² from hour 24 to hour 32,
40 μg/cm² to 60 μg/cm² from hour 32 to hour 48,
50 μg/cm² to 80 μg/cm² from hour 48 to hour 72,
60 μg/cm² to 100 μg/cm² from hour 72 to hour 144, and
15 μg/cm² to 30 μg/cm² from hour 144 to hour 168.

The commercial product Norspan® provides a non-cumulative skin permeation rate of buprenorphine base as measured in a Franz diffusion cell in the same setting of

3.19 μg/cm² in the first 8 hours,
22.40 μg/cm² from hour 8 to hour 24,
13.83 μg/cm² from hour 24 to hour 32,
26.17 μg/cm² from hour 32 to hour 48,
32.43 μg/cm² from hour 48 to hour 72,
60.10 μg/cm² from hour 72 to hour 144, and
17.17 μg/cm² from hour 144 to hour 168.

### Method of Manufacture

According to one further aspect, the invention relates to a method of manufacture of a transdermal therapeutic system for the transdermal administration of buprenorphine, comprising the steps of
1. providing a buprenorphine-containing composition comprising
   a) a polymer (e.g., polysiloxane)
   b) buprenorphine base or a pharmaceutically acceptable salt thereof
   c) a carboxylic acid (e.g., levulinic acid), and
   d) solvent (e.g., heptane and ethanol);
2. coating said buprenorphine-containing composition on a film (e.g., polyethylene terephthalate film) in an amount to provide the desired coating dry weight,
3. drying said coated buprenorphine-containing composition to provide a buprenorphine-containing matrix layer with the desired coating dry weight,
4. laminating said buprenorphine-containing matrix layer to a backing layer (e.g., Scotchpak 1220 from 3M),
5. providing an adhesive composition comprising a polymer-based pressure-sensitive adhesive,
6. coating said adhesive composition on a film in an amount to provide the desired coating dry weight,
7. drying said coated adhesive composition to provide a skin contact layer with the desired coating dry weight,
8. removing said film from the buprenorphine-containing matrix layer of step 4 and laminating said buprenorphine-containing matrix layer to said skin contact layer of step 7 to provide the buprenorphine-containing self-adhesive layer structure,
9. punching the individual systems from the buprenorphine-containing self-adhesive layer structure with the desired area of release, and
10. optionally adhering to the individual systems an active agent-free self-adhesive layer structure comprising also a backing layer and an active agent-free pressure-sensitive adhesive layer larger than the individual systems of the buprenorphine-containing self-adhesive layer structure.

In step 1 of said method of manufacture, preferably buprenorphine base and levulinic acid are used and are suspended in ethanol and subsequently combined with the polymer, preferably with polysiloxane in heptane to provide the buprenorphine-containing composition.

### EXAMPLES

The present invention will now be more fully described with reference to the accompanying examples. It should be understood, however, that the following description is illustrative only and should not be taken in any way as a restriction of the invention.

### Example 1

The composition of the buprenorphine base-containing adhesive solution is summarized in Table 1a below and the composition of the active-agent-free skin contact layer is summarized in Table 1b below.

TABLE 1a

| | Amt/unit |
|---|---|
| Ingredient (Trade Name) | (kg) |
| Buprenorphine base | 0.42 |
| Levulinic acid | 0.56 |
| Ethanol | 0.28 |
| Polysiloxane adhesive in n-heptane | 6.25 |
| Solids content of 74% by weight | |
| (BIO-PSA 7-4201 from Dow Corning | |
| Healthcare) | |
| n-heptane | 0.49 |
| Total | 8.00 |

TABLE 1b

| | Amt/unit |
|---|---|
| Ingredient (Trade Name) | (kg) |
| Polyacrylate adhesive prepared from 2- | 3.69 |
| ethylhexyl acrylate, vinyl acetate and 2- | |
| hydroxyethyl acrylate in Ethyl acetate | |
| Solids content 50.5% | |
| Ethyl acetate | 1.64 |
| Total | 5.33 |

In a stainless steel vessel, 0.42 kg of buprenorphine were suspended in 0.56 kg of levulinic acid and 0.28 kg of ethanol. With stirring, 6.25 kg of a polysiloxane adhesive in the form of a solution in n-heptane having a solids content of 74% by weight and 0.49 kg of heptane were added. The mixture was stirred until the buprenorphine base was fully dissolved, to give 8.00 kg of a buprenorphine-containing adhesive solution with 5.25% of buprenorphine, with a solids content of 70% (buprenorphine base-containing adhesive solution).

For the skin contact layer, a polyacrylate adhesive prepared from 2-ethylhexyl acrylate, vinyl acetate and 2-hydroxyethyl acrylate were used. 3.69 kg of a solution of this adhesive, with a solids content of 50.5% by weight, was admixed with 1.64 kg of ethyl acetate, following homogenization resulting in 5.33 kg of active-agent-free polyacrylate solution with a solids content of 35% (buprenorphine base-free adhesive solution)

The buprenorphine base-containing adhesive solution was coated on an adhesive polyethylene terephthalate film (e.g., Scotchpak from 3M) using an Erichsen coater and the solvent was removed by drying at approximately 50° C. for about 10 minutes to provide the buprenorphine base-containing matrix layer. The coating thickness was chosen such that removal of the solvents results in a coating weight of the buprenorphine base-containing matrix layer of 60 g/m². This results in the 7.5% by weight of buprenorphine base and 10% by weight of levulinic acid in this buprenorphine base-containing matrix layer. The dried film was laminated with the backing layer (e.g Scotchpak from 3M).

The active-agent-free polyacrylate adhesive solution was likewise coated onto an adhesively treated film (the later protective film to be removed before the systems are used) and the organic solvents were removed to produce the skin contact layer. The coating thickness of the resulting skin contact layer ought to amount, following removal of the solvents, to approximately 20 g/m². The adhesively treated film was then removed from the buprenorphine base-containing matrix layer produced first, and the buprenorphine base-containing matrix layer was laminated onto the skin contact layer.

The individual systems (TTS) were then punched from the buprenorphine-containing self-adhesive layer structure. In specific embodiments a TTS as described above can be provided with a further self-adhesive layer of larger surface area, preferably with rounded corners, comprising a pressure-sensitive adhesive matrix layer which is free of active ingredient and has a preferably skin-colored backing layer. This is of advantage when the TTS, on the basis of its physical properties alone, does not adhere sufficiently to the skin and/or when the buprenorphine-containing matrix layer, for the purpose of avoiding waste, has pronounced corners (square or rectangular shapes). The plasters are then punched out and sealed into pouches of the primary packaging material.

### Example 2

The composition of the buprenorphine base-containing adhesive solution is summarized in Table 2a below and the composition of the active-agent-free skin contact layer is summarized in Table 2b below.

TABLE 2a

| | Amt/unit |
|---|---|
| Ingredient (Trade Name) | (g) |
| Buprenorphine base | 1.88 |
| Levulinic acid | 2.50 |
| Ethanol | 2.00 |
| Polysiloxane adhesive in n-heptane | 27.87 |
| Solids content of 73% by weight | |
| (BIO-PSA 7-4301 from Dow Corning | |
| Healthcare) | |
| n-heptane | 1.00 |
| Total | 35.25 |

TABLE 2b

| | Amt/unit |
|---|---|
| Ingredient (Trade Name) | (g) |
| Polyacrylate adhesive prepared from 2- | 69.3 |
| ethylhexyl acrylate, vinyl acetate and 2- | |
| hydroxyethyl acrylate in Ethyl acetate | |
| Solids content 50.5% | |
| Ethyl acetate | 30.7 |
| Total | 100.0 |

The process of manufacture was as described for Example 1. The coating thickness was also chosen such that removal of the solvents results in a coating weight of the matrix layer of 60 g/m² and thus resulted in 7.5% by weight buprenorphine base and 10% by weight levulinic acid in this buprenorphine base-containing matrix layer.

### Example 3

The composition of the buprenorphine base-containing adhesive solution is summarized in Table 3a below and the composition of the active-agent-free skin contact layer is summarized in Table 3b below.

TABLE 3a

| | Amt/unit |
|---|---|
| Ingredient (Trade Name) | (g) |
| Buprenorphine base | 3.00 |
| Levulinic acid | 3.60 |
| Ethanol | 2.00 |
| Polysiloxane adhesive in n-heptane | 45.14 |
| Solids content of 73% by weight | |
| (BIO-PSA 7-4301 from Dow Corning | |
| Healthcare) | |
| n-heptane | 4.50 |
| Total | 58.24 |

TABLE 3b

| | Amt/unit |
|---|---|
| Ingredient (Trade Name) | (g) |
| Polyacrylate adhesive prepared from 2- | 69.3 |
| ethylhexyl acrylate, vinyl acetate and 2- | |
| hydroxyethyl acrylate in Ethyl acetate | |
| Solids content 50.5% | |
| Ethyl acetate | 30.7 |
| Total | 100.0 |

The process of manufacture was as described for Example 1. The coating thickness was also chosen such that removal of the solvents results in a coating weight of the matrix layer of 60 g/m² and thus resulted in 7.5% by weight buprenorphine base and 9% by weight levulinic acid in this buprenorphine base-containing matrix layer.

### Example 4

In Example 4 the in-vitro releases and the corresponding skin permeation rates of Examples 1 to 3 and Norspan® were determined by in vitro experiments in accordance with the OECD Guideline (adopted Apr. 13, 2004) carried out with a 9 ml Franz diffusion cell. Split thickness human skin from cosmetic surgeries (female breast, date of birth 1989) was used. A dermatome was used to prepare skin to a thickness of 800 μm, with an intact epidermis for all examples 1 to 3 and the commercial product Norspan®. Diecuts with an area of 1.163 cm² were punched from examples 1 to 3, and were each tested against diecuts of the commercial product Norspan®. The concentrations of buprenorphine in the receptor medium of the Franz cell (phosphate buffer solution pH 5.5 with 0.1% saline azide as antibacteriological agent) at a temperature of 32±1° C. were measured. The results are shown in Tables 4.1 to 4.5 and FIGS. 1 and 2.

TABLE 4.1
Non-cumulative release [μg/cm²] n = 3 (SD)

| Elapsed | | | | |
|---|---|---|---|---|
| time (hr) | Example 1 | Example 2 | Example 3 | Norspan ® |
| 0 | 0 | 0 | 0 | 0 |
| 8 | 2.12 | 3.23 | 2.60 | 3.19 |
| | (1.44) | (0.75) | (1.98) | (0.77) |
| 24 | 28.60 | 31.33 | 22.23 | 22.40 |
| | (10.19) | (7.71) | (7.95) | (3.76) |
| 32 | 26.37 | 24.80 | 18.33 | 13.83 |
| | (6.47) | (4.76) | (5.54) | (2.32) |
| 48 | 53.03 | 49.17 | 42.40 | 26.17 |
| | (5.80) | (5.89) | (9.69) | (2.46) |
| 72 | 58.47 | 58.87 | 60.70 | 32.43 |
| | (2.42) | (1.36) | (6.84) | (2.23) |
| 144 | 73.27 | 83.23 | 84.50 | 60.10 |
| | (4.63) | (3.09) | (1.76) | (2.02) |
| 168 | 17.87 | 21.00 | 20.67 | 17.17 |
| | (1.35) | (0.96) | (0.74) | (1.72) |

TABLE 4.2
Mean non-cumulative skin permeation rate [μg/cm²-hr] n = 3 (SD)

| | Sample | | | | |
|---|---|---|---|---|---|
| Elapsed | interval | | | | |
| time (hr) | (hr) | Example 1 | Example 2 | Example 3 | Norspan ® |
| 0 | 0 | 0 | 0 | 0 | 0 |
| 8 | 8 | 0.27 | 0.40 | 0.33 | 0.40 |
| | | (0.18) | (0.09) | (0.25) | (0.10) |
| 24 | 16 | 1.79 | 1.96 | 1.39 | 1.40 |
| | | (0.64) | (0.48) | (0.50) | (0.24) |
| 32 | 8 | 3.30 | 3.10 | 2.29 | 1.73 |
| | | (0.81) | (0.60) | (0.69) | (0.29) |
| 48 | 16 | 3.31 | 3.07 | 2.65 | 1.64 |
| | | (0.36) | (0.37) | (0.61) | (0.15) |
| 72 | 24 | 2.44 | 2.45 | 2.53 | 1.35 |
| | | (0.10) | (0.06) | (0.29) | (0.09) |
| 144 | 72 | 1.02 | 1.16 | 1.17 | 0.83 |
| | | (0.06) | (0.04) | (0.02) | (0.03) |
| 168 | 24 | 0.74 | 0.88 | 0.86 | 0.72 |
| | | (0.06) | (0.04) | (0.03) | (0.07) |

TABLE 4.3
Mean non-cumulative skin permeation rate
[μg/cm²-hr] n = 3 (SD) and per area of release [μg/hr]

| | | Area | | | | Norspan ® |
|---|---|---|---|---|---|---|
| Elapsed | Sample | of | | | | Area of |
| time | interval | release | Exam- | | Exam- | release |
| (hr) | (hr) | (cm²) | ple 1 | Example 2 | ple 3 | (25 cm²) |
| 0 | 0 | | 0 | 0 | 0 | 0 |
| 8 | 8 | | 0.27 | 0.40 | 0.33 | 0.40 |
| | | | (0.18) | (0.09) | (0.25) | (0.10) |
| | | 10 | 2.65 | 4.04 | 3.25 | 9.97 |
| | | 15 | 3.98 | 6.06 | 4.88 | 9.97 |
| | | 18.75 | 4.98 | 7.58 | 6.09 | 9.97 |
| 24 | 16 | | 1.79 | 1.96 | 1.39 | 1.40 |
| | | | (0.64) | (0.48) | (0.50) | (0.24) |
| | | 10 | 17.88 | 19.58 | 13.90 | 35.00 |
| | | 15 | 26.81 | 29.38 | 20.84 | 35.00 |
| | | 18.75 | 33.52 | 36.72 | 26.05 | 35.00 |
| 32 | 8 | | 3.30 | 3.10 | 2.29 | 1.73 |
| | | | (0.81) | (0.60) | (0.69) | (0.29) |
| | | 10 | 32.96 | 31.00 | 22.92 | 43.23 |
| | | 15 | 49.44 | 46.50 | 34.38 | 43.23 |
| | | 18.75 | 61.80 | 58.13 | 42.97 | 43.23 |
| 48 | 16 | | 3.31 | 3.07 | 2.65 | 1.64 |
| | | | (0.36) | (0.37) | (0.61) | (0.15) |
| | | 10 | 33.15 | 30.73 | 26.50 | 40.89 |
| | | 15 | 49.72 | 46.09 | 39.75 | 40.89 |
| | | 18.75 | 62.15 | 57.62 | 49.69 | 40.89 |
| 72 | 24 | | 2.44 | 2.45 | 2.53 | 1.35 |
| | | | (0.10) | (0.06) | (0.29) | (0.09) |
| | | 10 | 24.36 | 24.53 | 25.29 | 33.78 |
| | | 15 | 36.54 | 36.79 | 37.94 | 33.78 |
| | | 18.75 | 45.68 | 45.99 | 47.42 | 33.78 |
| 144 | 72 | | 1.02 | 1.16 | 1.17 | 0.83 |
| | | | (0.06) | (0.04) | (0.02) | (0.03) |
| | | 10 | 10.18 | 11.56 | 11.74 | 20.87 |
| | | 15 | 15.26 | 17.34 | 17.60 | 20.87 |
| | | 18.75 | 19.08 | 21.68 | 22.01 | 20.87 |
| 168 | 24 | | 0.74 | 0.88 | 0.86 | 0.72 |
| | | | (0.06) | (0.04) | (0.03) | (0.07) |
| | | 10 | 7.44 | 8.75 | 8.61 | 17.88 |
| | | 15 | 11.17 | 13.13 | 12.92 | 17.88 |
| | | 18.75 | 13.96 | 16.41 | 16.15 | 17.88 |

TABLE 4.4
Cumulative release after 168 hours of release [μg/cm²] n = 3

| Example 1 | Example 2 | Example 3 | Norspan ® |
|---|---|---|---|
| 259.72 | 271.63 | 251.43 | 175.29 |

TABLE 4.5
Mean cumulative skin permeation rate over 168 hours [μg/cm²-hr]

| Example 1 | Example 2 | Example 3 | Norspan ® |
|---|---|---|---|
| 1.55 | 1.62 | 1.50 | 1.04 |

### Example 5

In Example 5, a pharmacokinetic study in healthy adult male and female subjects was conducted as part of a 2 stage, randomised, open-label, single-dose, 4-part crossover design pharmacokinetic study to assess the pharmacokinetics and potential of Example 1 TTS formulations for equivalence to the existing commercial formulation BuTrans®, also known as Norspan®.

The study treatments were as follows:

Test treatment: Example 1 TTS (the amount of buprenorphine base being 6.75 mg; the area of release being 15 cm²)—applied for 7 consecutive days.
Reference treatment: BuTrans® 20 μg/hr (the amount of buprenorphine base being 20 mg; the area of release being 25 cm²)—applied for 7 consecutive days.

Further study treatments were administered in the 2 stage study but are not described herein.

The treatments were each worn over a 7-day period. Each subject was randomised to both the order, and TTS site of the treatments to be delivered over the study periods.

As this study was conducted in healthy human subjects, the opioid antagonist naltrexone was co-administered to reduce opioid-related adverse events. 50 mg naltrexone were administered with 100 ml of water every 12 hours beginning −13 hours prior to TTS application and continuing until 215 hours post-TTS application.

### Subject Selection

### Number of Subjects

It was anticipated that approximately 32 subjects would be randomized into stage 1 of the study, with 26 subjects targeted to complete stage 1 of the study. An adequate number of subjects were screened in the pre-treatment phase, i.e. within 21 days prior to the treatment phase to achieve this sample size.

### Screening Procedure

Screening procedures were performed for all potential subjects at a screening visit conducted within 21 days prior to the treatment phase, i.e. prior to Day −1 of study period 1. The following evaluations were performed after the subject has signed the study specific consent form:
Inclusion/Exclusion criteria
Demography (sex, date of birth, race) and body mass index (BMI)
Medical history (including confirmation of eligibility from the subject's primary care physician)
Physical examination including height, weight, and body mass index
Haematology (haemoglobin, red blood cell count, haematocrit, platelets, white blood cell count and differential (neutrophils, lymphocytes, monocytes, eosinophils and basophils))
Blood Chemistry (sodium, calcium, potassium, bicarbonate, chloride, urea, creatinine, uric acid, albumin, total protein, alkaline phosphatase, globulin, aspartate aminotransferase, alanine aminotransferase, gamma glutamyl-transferase, total bilirubin, direct bilirubin, glucose, inorganic phosphate, lactate dehydrogenase, triglyceride and cholesterol)
Urinalysis (specific gravity, pH, protein, ketone, occult blood, glucose; and additional microscopy analysis will be undertaken if any abnormalities are detected to analyse for red blood cells, white blood cells, epithelial cells, bacteria, casts, and crystals)
Urine drugs of abuse (opiates, cocaine metabolites, barbiturates, amphetamines, methadone, benzodiazepines, phencyclidine, methamphetamine, tricyclic antidepressants and cannabinoids) and alcohol test (urine or breath)
Serology testing (Human immunodeficiency virus (HIV), Hepatitis B surface antigen (HBsAg), Hepatitis C antibody)
12-lead Electrocardiogram (ECG)
Serum pregnancy test for females of child-bearing potential
Serum FSH for post-menopausal females
Vital signs (Pulse oximetry/oxygen saturation (SpO₂), supine respiration rate, supine blood pressure, supine pulse rate and oral temperature)
Medication history and concomitant medications will also be recorded.

### Inclusion Criteria

Subjects who met the following criteria were included in the study.
1. Provide written informed consent.
2. Healthy male or female subjects aged 18 to 55 inclusive.
3. Female subjects who are sexually active or become sexually active must be willing to use highly effective methods of contraception throughout the study. A highly effective method of birth control is defined as one which results in a low failure rate (i.e. less than 1% per year) when used consistently and correctly such as sterilisation, implants, injectables, combined oral contraceptives, some IUDs (Intrauterine Device), or vasectomised partner.
4. Female subjects including those up to 1 year post-menopausal must have a negative serum pregnancy test.
5. Female subjects who have been post-menopausal for >1 year and have elevated serum follicle-stimulating hormone (FSH) or are treated with hormone replacement therapy (HRT).
6. Male subjects who are willing to use contraception with their partners throughout the study and for 10 days after completion of the study and agree to inform the Investigator if their partner becomes pregnant during this time.
7. Body weight ranging from 55 to 100 kg and a BMI≥18 and ≤29.
8. Healthy and free of significant abnormal findings as determined by medical history, physical examination, vital signs, laboratory tests and ECG.
9. Willing to eat all the food supplied throughout the study.
10. The subject's primary care physician has confirmed within the last 12 months that there is nothing in the subject's medical history that would preclude their enrolment into a clinical study.
11. Will refrain from strenuous exercise during the entire study. They will not begin a new exercise program nor participate in any unusually strenuous physical exertion.

### Exclusion Criteria

The following criteria excluded potential subjects from the study.
1. Female subjects who are pregnant or lactating.
2. Any history of drug or alcohol abuse.
3. Any history of conditions that might interfere with drug absorption, distribution, metabolism or excretion.
4. Use of opioid or opioid antagonist-containing medication in the past 30 days.
5. Any history of frequent nausea or vomiting regardless of aetiology.
6. Any history of seizures or symptomatic head trauma.
7. Participation in a clinical drug study during the 90 days preceding the initial dose in this study or participation in any other study during this study.
8. Any significant illness during the 4 weeks preceding entry into this study.
9. A history of additional risk factors for Torsades de Pointes (e.g. heart failure, hypokalaemia, personal or family history of long QT syndrome, syncope, or family history of sudden death).
10. Abnormal cardiac conditions including any of the following:
   QTc interval greater than 450 msec at screening or at check-in before first dosing.
   Increase in QTc of more than 60 msec above pre-dose values of each study period.
11. Use of medication within 5 times the half-life or minimum 14 days for prescription medication or 7 days for over-the-counter preparations (including vitamins, herbal and/or mineral supplements), whichever is longer, before the first dose of study treatment and during the study (with the exception of the continued use of HRT and contraceptives). Note: subjects taking oral contraceptives containing CYP3A4 inhibitors such as gestodene should be excluded as this may lead to elevated plasma concentrations.
12. Refusal to abstain from caffeine or xanthine containing beverages entirely until the last study PK sample has been taken.
13. Weekly alcohol intake exceeding the equivalent of 14 units/week for females and 21 units/week for males.
14. Consumption of alcoholic beverages within 48 hours before study drug administration, and refusal to abstain from alcohol for the duration of the study confinement and for at least 72 hours after the last naltrexone dose.
15. History of smoking within 45 days of study drug administration and refusal to abstain from smoking during the study.
16. Blood or blood products donated within 90 days prior to study drug administration or any time during the study, except as required by this protocol.
17. Positive results of urine drug screen, alcohol test, pregnancy test, HBsAg, Hepatitis C antibody, or HIV tests.
18. Known hypersensitivity or sensitivity to buprenorphine, naltrexone or related compounds or any of the excipients or any contraindications as detailed in the Summary of Product Characteristics.
19. Clinically significant history of allergic reaction to wound dressings or elastoplast.
20. Subjects with tattoos or any dermatological disorder at the proposed sites of TTS application, or with a history of eczema/cutaneous atrophy.
21. Subjects who will not allow hair to be removed at the proposed TTS application sites which may prevent proper placement of the TTS.
22. Refusal to allow their primary care physician to be informed.

Subjects meeting all the inclusion criteria and none of the exclusion criteria were randomized into the study.

### Treatment Phase Procedures

### Randomisation

Randomisation was completed once all inclusion and exclusion criteria are verified. Randomisation order was determined on a central randomisation list held at site (one list per site).

Subjects were randomised to the order of the treatments and the skin TTS application sites.

There are 4 possible TTS application sites:
Deltoid region of the non-dominant arm
Deltoid region of the dominant arm
Right upper back
Left upper back.

### Check-in Procedures

On each day prior to treatment (e.g. Day −1 or Day 17), subjects were checked in to the study unit. The following procedures were undertaken:
Review of consent and eligibility
Urine pregnancy test (Female subjects of child bearing potential only)
Alcohol screen (by breath test) and
Urine drug screen as per screening visit
Naltrexone HCl dosing
Adverse events
Concomitant medications will be recorded.

### Randomisation Occurred Once in the Study on Day −1.

### Study Procedures

The treatment phase included study periods with a single dose application. The following procedures were undertaken in each period:
Pre-dosing biochemistry (fasting) as per screening
TTS application
Vital signs (supine respiration rate, supine blood pressure, supine pulse rate)
SpO₂
Blood samples for drug concentration measurements obtained pre-dose and at pre-specified times throughout the duration of the study for each subject; TTS was removed at 168 hours after TTS application; blood draw must be performed immediately prior to TTS removal
12-lead ECG (taken before each TTS application, at 72, 120, and 168 hours after each TTS application in each study period and at the Post-Study Medical)
Oral temperature was recorded at specified times throughout the study
Adverse events; recorded throughout the study on an ongoing basis whilst confined to the study unit and through open questioning. Any recorded skin reactions will also be recorded as adverse events.
Concomitant medications; recorded at Screening and throughout the study
TTS site skin assessment and duration and observation assessments; duration of TTS wear assessments were rated just after application and then at the same time each day of TTS wear. TTS observation assessments were performed just before TTS removal. Skin site reaction will be assessed 30 min after TTS removal.

Where more than one procedure was scheduled at the same time-point, the following order of procedures was ideally followed:
BTDS blood sample collection within ±5 minutes of scheduled sampling time post dose. Pre-dose sample must be taken within the hour before study drug dosing
Vital signs and ECG (within ±15 minutes of scheduled time)
Pulse oximetry (within ±15 minutes of scheduled time)
Skin reaction assessment at application site (within ±5 minutes of scheduled time)
Duration of TTS wear observations (within ±30 minutes of scheduled time)
Observation of TTS at removal (within −30 minutes of scheduled time)
Food and fluids (start time within ±30 minutes of scheduled time).

Throughout the Study Period when subjects had the TTS applied, they were allowed to have a shower (not bath) but they had to refrain from washing, or rubbing the site of TTS application. The subjects should also refrain from showering until the day after TTS application. The TTS was removed on the eighth day of the Study Period following the blood draw at 168 hours after TTS application.

### Washout Period

There was a minimum 10 day washout period between removal of one TTS and application of another.

### Confinement to the Study Unit

Subjects were confined to the study unit from Check-In on the day before study drug administration until the time that the 192 hour post-TTS application procedures were completed. Subjects returned to the unit for the 216, 240, 264 and 288 hours post-study procedures and the Post-Study Medical. During confinement in the unit, subjects will receive standardised meals.

### Pharmacokinetic Measurements

Blood samples for pharmacokinetic assessments were obtained for each subject at predose and at 2, 4, 8, 12, 16, 24, 36, 48, 60, 72, 84, 96, 108, 120, 144, 168, 169, 172, 176, 180, 192, 216, 240, 264 and 288 hours post-TTS application.

For each sample, 4 ml of blood were drawn into 4 ml tubes containing K₂EDTA solution, an anticoagulant. Samples were centrifuged within 30 minutes of collection. Following centrifugation (1500 G, 4° C., 15 minutes), the plasma was transferred, via pipette, into 2 labelled 3 ml polypropylene tubes, and stored at −20° C. within 1 hour of collection.

Plasma concentrations of analytes were quantified by liquid chromatography—tandem mass spectrometric methodology (LC-MS/MS) using a previously validated assay.

For each subject, the following pharmacokinetic parameters were calculated based on the plasma concentrations of buprenorphine:
AUCt (pg·hr/ml)—the area under the plasma concentration-time curve from hour 0 to the last measurable plasma concentration, calculated by the linear trapezoidal method;
AUCINF (pg·hr/ml)—the area under the plasma concentration-time curve extrapolated to infinity, calculated using the formula

$$AUCINF = AUCt + \frac{CLast}{LambdaZ} ,$$

where CLast is the last measurable plasma concentration and LambdaZ is the apparent terminal phase rate constant;
Cmax (pg/ml)—the maximum observed plasma concentration;
tmax (hr)—the time to maximum plasma concentration;
LambdaZ (1/hr)—the apparent terminal phase rate constant, where LambdaZ is the magnitude of the slope of the linear regression of the log concentration versus time profile during the terminal phase;
t½Z (hr)—the apparent plasma terminal phase half-life (whenever possible), where t½Z=(ln 2)/LambdaZ.

Plasma concentration values below the level of quantitation were set to equal zero for the analysis.

AUC values were calculated using the linear trapezoidal method. After removal of the BTDS, where possible, LambdaZ values were estimated using those points determined to be in the terminal log-linear phase. t½Z was determined from the ratio of ln 2 to LambdaZ.

### Individual Subject Stopping Criteria

Subjects who met one or more of the following stopping criteria were discontinued from the study:
Markedly Abnormal Liver Function Tests or Creatinine test
O₂ saturation 85% or less
Increase in QTc of more than 60 msec above pre-dose values of each study period or QTc greater than 500 msec
Serious adverse drug reaction
Severe nausea and vomiting
Severe reaction at TTS site or a local reaction which necessitates removal of the TTS or discontinuation of the infusion
Systolic blood pressure (BP)≥180 mmHg
Heart rate (HR)≥140 bpm
Other BP and HR values and changes from baseline if associated with cardiovascular compromise.

### Study Restrictions

As per the inclusion/exclusion criteria, subjects had to be willing to eat all the food supplied throughout the study. Menus were standardised while subjects are in the study unit. The menus were the same for each study period. However, the menus for each day needed not be identical. Subjects had to consume only the food given to them while in the unit. Food and water will be restricted as follows:
Subjects were given an evening meal and snack following check-in to the study unit on the day before dosing to be consumed >8 hours before dosing.
Subjects received a light breakfast 1 hour before commencement of treatment. There was free access to drinking water throughout the day, except within 30 minutes before vital sign measurements or commencement of treatment. A low fat lunch (<30% fat), dinner, and an evening snack were provided at 4, 10, and 14 hours after TTS application. Drinks of decaffeinated tea or decaffeinated coffee were supplied with meals.
Meals were provided at the same time each day (as on Day 1). There was free access to drinking water and de-caffeinated drinks throughout the day, except within 30 minutes before vital sign measurements.
Breakfast will be optional after all study procedures have been completed.

Subjects had to abstain from smoking within 45 days of study drug administration and during the entire study. Subjects had to abstain from alcohol from 48 hours before the first study drug administration until 72 hours after the last naltrexone dose of the last study period. Caffeine or xanthine containing food or beverages were not permitted during the study from check-in before treatment, until after the last study pharmacokinetic sample has been taken.

### Follow-Up Period

Subjects that completed the treatment phase or who discontinued treatment early were followed up within 7 to 10 days after the Subject's last visit/dose of study medication.

Study Completion Procedures

Subjects that completed the Treatment Phase carried out the following Completion/Discontinuation Visit procedures:
Subjects attended a Post-Study Medical Visit 7 to10 days after removal of their last TTS if this was the last treatment received in the case of completion/discontinuation from the study.
Safety was monitored and Post-Study Medical procedures were carried out including the following:
   Physical examination including weight measurement
   Haematology (as for screening visit)
   Blood chemistry (as for screening visit)
   Urinalysis (as for screening visit)
   Serum pregnancy test for females of child bearing potential
   12-lead ECG
   Vital signs (supine respiration rate, supine blood pressure, supine pulse rate)
   Pulse oximetry
   Oral temperature
   Review of adverse events
   Review of concomitant therapy.

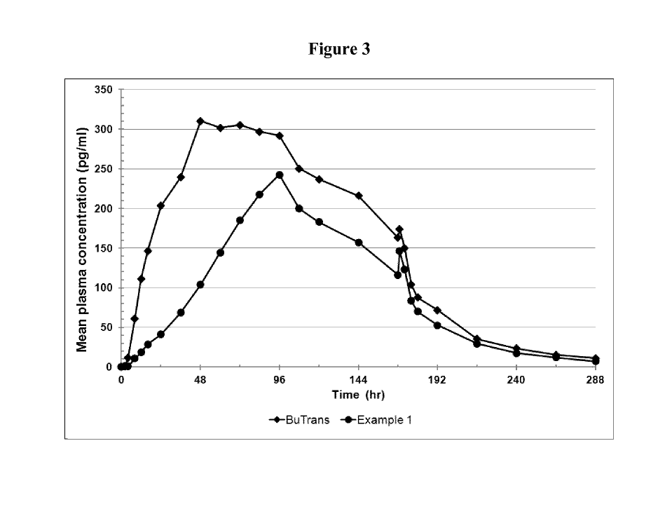

The results of this study are shown in FIG. 3 and Tables 5.1 to 5.5 below.

TABLE 5.1
Statistical results for pharmacokinetic parameters (full analysis
population): Example 1 TTS (6.75 mg) relative to BuTrans ® (20 mg)

| | Example 1 | | Example 1 | |
|---|---|---|---|---|
| | TTS | BuTrans ® | TTS | BuTrans ® |
| | Cmax (pg/ml) | | AUCt (pg · hr/ml) | |
| n^{a} | 28 | 28 | 28 | 28 |
| Mean^{b} | 288.29 | 383.63 | 27709.30 | 44323.44 |
| SD^{c} | 137.67 | 176.63 | 13213.42 | 19273.58 |
| SE^{d} | 26.02 | 33.38 | 2497.10 | 3642.36 |
| GeoMean^{e} | 258.05 | 346.47 | 25025.91 | 40613.23 |
| log SD^{f} | 0.484 | 0.467 | 0.456 | 0.428 |
| log SE^{g} | 0.091 | 0.088 | 0.086 | 0.081 |
| Min^{h} | 111.98 | 120.03 | 11539.6 | 14312.1 |
| Medianⁱ | 254.25 | 376.74 | 24401.87 | 40866.71 |
| Max^{k} | 595.80 | 872.38 | 57931.7 | 100315.6 |
| | AUCINF (pg · hr/ml) | | tmax (hr) | |
| n^{a} | 26 | 25 | 28 | 28 |
| Mean^{b} | 28850.38 | 45108.89 | 108.21 | 81.93 |
| SD^{c} | 13805.37 | 19782.01 | 38.02 | 37.56 |
| SE^{d} | 2707.46 | 3956.40 | 7.19 | 7.10 |
| GeoMean^{e} | 26019.04 | 41273.54 | NA^{l} | NA^{l} |
| log SD^{f} | 0.461 | 0.434 | NA^{l} | NA^{l} |
| log SE^{g} | 0.090 | 0.087 | NA^{l} | NA^{l} |
| Min^{h} | 11702.00 | 14619.5 | 48.00 | 24.00 |
| Medianⁱ | 25186.06 | 43282.61 | 96.00 | 72.00 |
| Max^{k} | 60731.70 | 101394.2 | 169.00 | 169.00 |
| | LambdaZ (1/hr) | | t1/2Z (hr) | |
| n^{a} | 26 | 25 | 26 | 25 |
| Mean^{b} | 0.0172 | 0.0175 | 50.38 | 44.73 |
| SD^{c} | 0.0090 | 0.0068 | 27.38 | 16.82 |
| SE^{d} | 0.0018 | 0.0014 | 5.37 | 3.36 |
| Min^{h} | 0.004 | 0.0070 | 13.80 | 16.75 |
| Medianⁱ | 0.0157 | 0.0164 | 44.14 | 42.22 |
| Max^{k} | 0.050 | 0.041 | 154.54 | 98.27 |

^{a}n = number of subjects with data available (non-zero values).
^{b}Mean = arithmetic mean; the sum of all the values of observations divided by the total number of observations.
^{c}SD = standard deviation.
^{d}SE = standard error.
^{e}GeoMean = geometric mean; the mean of the log transformed data backtransformed to the original scale.
^{f}log SD = standard deviation of the log transformed data.
^{g}log SE = standard error of the log transformed data.
^{h}Min = minimum value.
ⁱMedian = middle value when the list of values is ranked.
^{k}Max = maximum value.
^{l}NA = not applicable.

TABLE 5.2
Summary of mixed model^{a} for pharmacokinetic parameters
Cmax, AUCt, and AUCINF(full analysis population): Example 1
TTS (6.75 mg) relative to BuTrans ® (20 mg)

| | | | | LS Mean^{c} | |
|---|---|---|---|---|---|
| | | | | Ratio | |
| | | LS Mean^{b} | | Exampel 1 | 90% |
| | | Example 1 | | TTS/ | Confidence |
| | n^{d} | TTS | BuTrans ® | BuTrans ® (%) | Interval (%) |
| Cmax | 26 | 274.03 | 348.94 | 78.53^{e} | [65.43, 94.26] |
| AUCt | 26 | 26037.56 | 41121.81 | 63.32^{f} | [52.64, 76.16] |
| AUCINF | 21 | 26782.27 | 41460.21 | 64.60^{f} | [51.62, 80.84] |

^{a}Data analysed using a mixed effects linear model with treatment, actual sequence and period as fixed effects and subject within sequence as random effect. The analyses only consider subjects who completed both periods of the respective treatment comparison.
^{b}Least square mean; back-transformed from log scale to linear scale.
^{c}Least square mean; back-transformed from difference on log scale to ratio on linear scale.
^{d}Number of subjects with data for both Example 1 TTS and BuTrans ® available.
^{e}equivalent to relative Cmax ratio.
^{f}equivalent to relative bioavailability.

TABLE 5.3
Summary of mixed model^{a} for pharmacokinetic parameter
t1/2Z (full analysis population): Example 1 TTS (6.75 mg)
relative to BuTrans ® (20 mg)

| | | LS Mean^{b} | | | |
|---|---|---|---|---|---|
| | | | | Exampel 1 | 90% |
| | | Example 1 | | TTS- | Confidence |
| | n^{c} | TTS | BuTrans ® | BuTrans ® | Interval |
| t1/2Z | 21 | 52.64 | 42.59 | 10.05 | [0.32, 19.78] |

^{a}Data analysed using a mixed effects linear model with treatment, actual sequence and period as fixed effects and subject within sequence as random effect. The analyses only consider subjects who completed both periods of the respective treatment comparison.
^{b}Least square mean.
^{c}Number of subjects with data for both Example 1 TTS and BuTrans ® available.

TABLE 5.4
Mean AUCt per area of
release (pg · hr/ml-cm²)

| Example 1 | |
|---|---|
| TTS | BuTrans ® |
| 1668.39 | 1624.53 |

TABLE 5.5
Bioequivalence assessment relative to BuTrans ® for a
50% increase^{a} in plasma concentrations for Example 1 TTS

| | Ratio Exampel 1 TTS/BuTrans ® | 90% Confidence |
|---|---|---|
| | (%) | Interval (%) |
| ln(Cmax) | 119.77 | [102.53; 139.91] |
| ln(AUCt) | 97.43 | [83.70; 113.41] |
| ln(AUCINF) | 101.14 | [85.04; 120.29] |

^{a}Calculated based on the individual subject data of Example 1 TTS (6.75 mg).

The invention relates in particular to the following further items:

1. Transdermal therapeutic system for the transdermal administration of buprenorphine, comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
   and optionally wherein the buprenorphine-containing self-adhesive layer structure contains said buprenorphine in an amount of less than 0.8 mg/cm² buprenorphine base or an equimolar amount of a pharmaceutically acceptable salt thereof.
   2. Transdermal therapeutic system in accordance with item 1, said buprenorphine-containing self-adhesive layer structure containing less than 0.7 mg/cm² of buprenorphine base or an equimolar amount of a pharmaceutically acceptable salt thereof.
   3. Transdermal therapeutic system in accordance with item 2, said buprenorphine-containing self-adhesive layer structure containing less than 0.6 mg/cm² of buprenorphine base or an equimolar amount of a pharmaceutically acceptable salt thereof.
   4. Transdermal therapeutic system in accordance with item 2, said buprenorphine-containing self-adhesive layer structure containing less than 0.55 mg/cm² of buprenorphine base or an equimolar amount of a pharmaceutically acceptable salt thereof.
   5. Transdermal therapeutic system in accordance with item 2, said buprenorphine-containing self-adhesive layer structure containing less than 0.5 mg/cm² of buprenorphine base or an equimolar amount of a pharmaceutically acceptable salt thereof.
   6. Transdermal therapeutic system in accordance with item 1, said buprenorphine-containing self-adhesive layer structure containing from about 0.2 mg/cm² to less than 0.8 mg/cm² of buprenorphine base or an equimolar amount of a pharmaceutically acceptable salt thereof.
   7. Transdermal therapeutic system in accordance with item 6, said buprenorphine-containing self-adhesive layer structure containing from about 0.2 mg/cm² to about 0.7 mg/cm² of buprenorphine base or an equimolar amount of a pharmaceutically acceptable salt thereof.
   8. Transdermal therapeutic system in accordance with item 6, said buprenorphine-containing self-adhesive layer structure containing from about 0.2 mg/cm² to about 0.6 mg/cm² of buprenorphine base or an equimolar amount of a pharmaceutically acceptable salt thereof.
   9. Transdermal therapeutic system in accordance with item 6, said buprenorphine-containing self-adhesive layer structure containing from about 0.2 mg/cm² to less than 0.55 mg/cm² of buprenorphine base or an equimolar amount of a pharmaceutically acceptable salt thereof.
   10. Transdermal therapeutic system in accordance with item 6, said buprenorphine-containing self-adhesive layer structure containing from about 0.2 mg/cm² to about 0.5 mg/cm² of buprenorphine base or an equimolar amount of a pharmaceutically acceptable salt thereof.
   11. Transdermal therapeutic system in accordance with item 6, said buprenorphine-containing self-adhesive layer structure containing from about 0.3 mg/cm² to about 0.5 mg/cm² of buprenorphine base or an equimolar amount of a pharmaceutically acceptable salt thereof.
   12. Transdermal therapeutic system in accordance with item 6, said buprenorphine-containing self-adhesive layer structure containing from about 0.4 mg/cm² to about 0.5 mg/cm² of buprenorphine base or an equimolar amount of a pharmaceutically acceptable salt thereof.
   13. Transdermal therapeutic system in accordance with any one of items 1 to 12, the amount of said buprenorphine contained in the transdermal therapeutic system ranging from
   about 1 mg to about 4 mg buprenorphine base or an equimolar amount of a pharmaceutically acceptable salt thereof, or
   about 3.5 mg to about 8 mg buprenorphine base or an equimolar amount of a pharmaceutically acceptable salt thereof, or
   about 6.5 mg to about 16 mg buprenorphine base or an equimolar amount of a pharmaceutically acceptable salt thereof, or
   about 11.5 mg to about 24 mg buprenorphine base or an equimolar amount of a pharmaceutically acceptable salt thereof, or
   about 15 mg to about 32 mg buprenorphine base or an equimolar amount of a pharmaceutically acceptable salt thereof.
   14. Transdermal therapeutic system in accordance with item 13, the amount of said buprenorphine contained in the transdermal therapeutic system ranging from
   about 1 mg to about 3.5 mg buprenorphine base or an equimolar amount of a pharmaceutically acceptable salt thereof, or
   about 3.5 mg to about 7 mg buprenorphine base or an equimolar amount of a pharmaceutically acceptable salt thereof, or
   about 6.5 mg to about 14 mg buprenorphine base or an equimolar amount of a pharmaceutically acceptable salt thereof, or
   about 11.5 mg to about 21 mg buprenorphine base or an equimolar amount of a pharmaceutically acceptable salt thereof, or
   about 15 mg to about 28 mg buprenorphine base or an equimolar amount of a pharmaceutically acceptable salt thereof.
   15. Transdermal therapeutic system in accordance with item 13, the amount of said buprenorphine contained in the transdermal therapeutic system ranging from
   about about 1 mg to about 3 mg buprenorphine base or an equimolar amount of a pharmaceutically acceptable salt thereof, or
   about 3.5 mg to about 6 mg buprenorphine base or an equimolar amount of a pharmaceutically acceptable salt thereof, or
   about 6.5 mg to about 12 mg buprenorphine base or an equimolar amount of a pharmaceutically acceptable salt thereof, or
   about 12.5 mg to about 18 mg buprenorphine base or an equimolar amount of a pharmaceutically acceptable salt thereof, or
   about 18.5 mg to about 24 mg buprenorphine base or an equimolar amount of a pharmaceutically acceptable salt thereof.
   16. Transdermal therapeutic system in accordance with any one of items 1 to 15, the size of said buprenorphine-containing matrix layer providing the area of release ranging from
   more than 4.8 cm² to about 8 cm², or
   more than 9.5 cm² to about 15 cm², or
   more than 19 cm² to about 30 cm², or
   more than 28.5 cm² to about 45 cm², or
   more than 38 cm² to about 60 cm².
   17. Transdermal therapeutic system in accordance with item 16, the size of said buprenorphine-containing matrix layer providing the area of release ranging from
   about 5 cm² to about 7 cm², or
   about 10 cm² to about 13 cm², or
   about 20 cm² to about 26 cm², or
   about 30 cm² to about 39 cm², or
   about 40 cm² to about 52 cm².
   18. Transdermal therapeutic system in accordance with item 16, the size of said buprenorphine-containing matrix layer providing the area of release ranging from
   about 5 cm² to about 6 cm², or
   about 10 cm² to about 12 cm², or
   about 20 cm² to about 24 cm², or
   about 30 cm² to about 36 cm², or
   about 40 cm² to about 48 cm².
   19. Transdermal therapeutic system in accordance with any one of items 1 to 12, the amount of said buprenorphine contained in the transdermal therapeutic system ranging from about 1 mg to about 4 mg buprenorphine base or an equimolar amount of a pharmaceutically acceptable salt thereof and the size of said buprenorphine-containing matrix layer providing the area of release ranging from more than 4.8 cm² to about 8 cm².
   20. Transdermal therapeutic system in accordance with any one of items 1 to 12, the amount of said buprenorphine contained in the transdermal therapeutic system ranging from about 3.5 mg to about 8 mg buprenorphine base or an equimolar amount of a pharmaceutically acceptable salt thereof and the size of said buprenorphine-containing matrix layer providing the area of release ranging from more than 9.5 cm² to about 15 cm².
   21. Transdermal therapeutic system in accordance with any one of items 1 to 12, the amount of said buprenorphine contained in the transdermal therapeutic system ranging from about 6.5 mg to about 16 mg buprenorphine base or an equimolar amount of a pharmaceutically acceptable salt thereof and the size of said buprenorphine-containing matrix layer providing the area of release ranging from more than 19 cm² to about 30 cm².
   22. Transdermal therapeutic system in accordance with any one of items 1 to 12, the amount of said buprenorphine contained in the transdermal therapeutic system ranging from about 11.5 mg to about 24 mg buprenorphine base or an equimolar amount of a pharmaceutically acceptable salt thereof and the size of said buprenorphine-containing matrix layer providing the area of release ranging from more than 28.5 cm² to about 45 cm².
   23. Transdermal therapeutic system in accordance with any one of items 1 to 12, the amount of said buprenorphine contained in the transdermal therapeutic system ranging from about 15 mg to about 32 mg buprenorphine base or an equimolar amount of a pharmaceutically acceptable salt thereof and the size of said buprenorphine-containing matrix layer providing the area of release ranging from more than 38 cm² to about 60 cm².
   24. Transdermal therapeutic system in accordance with any one of items 1 to 12, the amount of said buprenorphine contained in the transdermal therapeutic system ranging from about 1 mg to about 3.5 mg buprenorphine base or an equimolar amount of a pharmaceutically acceptable salt thereof and the size of said buprenorphine-containing matrix layer providing the area of release ranging from about 5 cm² to about 7 cm².
   25. Transdermal therapeutic system in accordance with any one of items 1 to 12, the amount of said buprenorphine contained in the transdermal therapeutic system ranging from about 3.5 mg to about 7 mg buprenorphine base or an equimolar amount of a pharmaceutically acceptable salt thereof and the size of said buprenorphine-containing matrix layer providing the area of release ranging from about 10 cm² to about 13 cm².
   26. Transdermal therapeutic system in accordance with any one of items 1 to 12, the amount of said buprenorphine contained in the transdermal therapeutic system ranging from about 6.5 mg to about 14 mg buprenorphine base or an equimolar amount of a pharmaceutically acceptable salt thereof and the size of said buprenorphine-containing matrix layer providing the area of release ranging from about 20 cm² to about 26 cm².
   27. Transdermal therapeutic system in accordance with any one of items 1 to 12, the amount of said buprenorphine contained in the transdermal therapeutic system ranging from about 11.5 mg to about 21 mg buprenorphine base or an equimolar amount of a pharmaceutically acceptable salt thereof and the size of said buprenorphine-containing matrix layer providing the area of release ranging from about 30 cm² to about 39 cm².
   28. Transdermal therapeutic system in accordance with any one of items 1 to 12, the amount of said buprenorphine contained in the transdermal therapeutic system ranging from about 15 mg to about 28 mg buprenorphine base or an equimolar amount of a pharmaceutically acceptable salt thereof and the size of said buprenorphine-containing matrix layer providing the area of release ranging from about 40 cm² to about 52 cm².
   29. Transdermal therapeutic system in accordance with any one of items 1 to 12, the amount of said buprenorphine contained in the transdermal therapeutic system ranging from about about 1 mg to about 3 mg buprenorphine base or an equimolar amount of a pharmaceutically acceptable salt thereof and the size of said buprenorphine-containing matrix layer providing the area of release ranging from about 5 cm² to about 6 cm².
   30. Transdermal therapeutic system in accordance with any one of items 1 to 12, the amount of said buprenorphine contained in the transdermal therapeutic system ranging from about 3.5 mg to about 6 mg buprenorphine base or an equimolar amount of a pharmaceutically acceptable salt thereof and the size of said buprenorphine-containing matrix layer providing the area of release ranging from about 10 cm² to about 12 cm².
   31. Transdermal therapeutic system in accordance with any one of items 1 to 12, the amount of said buprenorphine contained in the transdermal therapeutic system ranging from about 6.5 mg to about 12 mg buprenorphine base or an equimolar amount of a pharmaceutically acceptable salt thereof and the size of said buprenorphine-containing matrix layer providing the area of release ranging from about 20 cm² to about 24 cm².
   32. Transdermal therapeutic system in accordance with any one of items 1 to 12, the amount of said buprenorphine contained in the transdermal therapeutic system ranging from about 12.5 mg to about 18 mg buprenorphine base or an equimolar amount of a pharmaceutically acceptable salt thereof and the size of said buprenorphine-containing matrix layer providing the area of release ranging from about 30 cm² to about 36 cm².
   33. Transdermal therapeutic system in accordance with any one of items 1 to 12, the amount of said buprenorphine contained in the transdermal therapeutic system ranging from about 18.5 mg to about 24 mg buprenorphine base or an equimolar amount of a pharmaceutically acceptable salt thereof and the size of said buprenorphine-containing matrix layer providing the area of release ranging from about 40 cm² to about 48 cm².
   34. Transdermal therapeutic system in accordance with any one of items 1 to 12, the amount of said buprenorphine contained in the transdermal therapeutic system ranging from about 1 mg to about 4 mg buprenorphine base or an equimolar amount of a pharmaceutically acceptable salt thereof.
   35. Transdermal therapeutic system in accordance with item 34, the amount of said buprenorphine contained in the transdermal therapeutic system ranging from about 1 mg to about 3.5 mg buprenorphine base or an equimolar amount of a pharmaceutically acceptable salt thereof.
   36. Transdermal therapeutic system in accordance with item 34, the amount of said buprenorphine contained in the transdermal therapeutic system ranging from about 1 mg to about 3 mg buprenorphine base or an equimolar amount of a pharmaceutically acceptable salt thereof.
   37. Transdermal therapeutic system in accordance with any one of items 34 to 36, the size of said buprenorphine-containing matrix layer providing the area of release ranging from more than 4.8 cm² to about 8 cm².
   38. Transdermal therapeutic system in accordance with item 37, the size of said buprenorphine-containing matrix layer providing the area of release ranging from about 5 cm² to about 7 cm².
   39. Transdermal therapeutic system in accordance with item 37, the size of said buprenorphine-containing matrix layer providing the area of release ranging from about 5 cm² to about 6 cm².
   40. Transdermal therapeutic system in accordance with any one of items 19, 24, 29, or 34 to 39, said transdermal therapeutic system providing a mean AUCt of more than 7,000 pg·hr/ml over about 168 hours of administration after a single-dose administration to a subject population.
   41. Transdermal therapeutic system in accordance with item 40, said transdermal therapeutic system providing a mean AUCt of more than 8,000 pg·hr/ml over about 168 hours of administration after a single-dose administration to a subject population.
   42. Transdermal therapeutic system in accordance with item 40, said transdermal therapeutic system providing a mean AUCt of from more than 8,000 pg·hr/ml to about 16,000 pg·hr/ml over about 168 hours of administration after a single-dose administration to a subject population.
   43. Transdermal therapeutic system in accordance with any one of items 19, 24, 29, or 34 to 42, said transdermal therapeutic system providing a mean release rate ranging from about 2.5 to about 7.5 μg/hr, and/or a nominal mean release rate of about 5 μg/hr over about 168 hours of administration.
   44. Transdermal therapeutic system in accordance with any one of items 1 to 12, the amount of said buprenorphine contained in the transdermal therapeutic system ranging from about 3.5 mg to about 8 mg buprenorphine base or an equimolar amount of a pharmaceutically acceptable salt thereof.
   45. Transdermal therapeutic system in accordance with item 44, the amount of said buprenorphine contained in the transdermal therapeutic system ranging from about 3.5 mg to about 7 mg buprenorphine base or an equimolar amount of a pharmaceutically acceptable salt thereof.
   46. Transdermal therapeutic system in accordance with item 44, the amount of said buprenorphine contained in the transdermal therapeutic system ranging from about 3.5 mg to about 6 mg buprenorphine base or an equimolar amount of a pharmaceutically acceptable salt thereof.
   47. Transdermal therapeutic system in accordance with any one of items 44 to 46, the size of said buprenorphine-containing matrix layer providing the area of release ranging from more than 9.5 cm² to about 15 cm².
   48. Transdermal therapeutic system in accordance with item 47, the size of said buprenorphine-containing matrix layer providing the area of release ranging from about 10 cm² to about 13 cm².
   49. Transdermal therapeutic system in accordance with item 47, the size of said buprenorphine-containing matrix layer providing the area of release ranging from about 10 cm² to about 12 cm².
   50. Transdermal therapeutic system in accordance with any one of items 20, 25, 30, or 44 to 49, said transdermal therapeutic system providing a mean AUCt of more than 14,000 pg·hr/ml over about 168 hours of administration after a single-dose administration to a subject population.
   51. Transdermal therapeutic system in accordance with item 50, said transdermal therapeutic system providing a mean AUCt of more than 16,000 pg·hr/ml over about 168 hours of administration after a single-dose administration to a subject population.
   52. Transdermal therapeutic system in accordance with item 50, said transdermal therapeutic system providing a mean AUCt of from more than 16,000 pg·hr/ml to about 32,000 pg·hr/ml over about 168 hours of administration after a single-dose administration to a subject population.
   53. Transdermal therapeutic system in accordance with any one of items 20, 25, 30, or 44 to 52, said transdermal therapeutic system providing a mean release rate ranging from about 8 to about 12 μg/hr, and/or a nominal mean release rate of about 10 μg/hr over about 168 hours of administration.
   54. Transdermal therapeutic system in accordance with any one of items 1 to 12, the amount of said buprenorphine contained in the transdermal therapeutic system ranging from about 6.5 mg to about 16 mg buprenorphine base or an equimolar amount of a pharmaceutically acceptable salt thereof.
   55. Transdermal therapeutic system in accordance with item 54, the amount of said buprenorphine contained in the transdermal therapeutic system ranging from about 6.5 mg to about 14 mg buprenorphine base or an equimolar amount of a pharmaceutically acceptable salt thereof.
   56. Transdermal therapeutic system in accordance with item 54, the amount of said buprenorphine contained in the transdermal therapeutic system ranging from about 6.5 mg to about 12 mg buprenorphine base or an equimolar amount of a pharmaceutically acceptable salt thereof.
   57. Transdermal therapeutic system in accordance with any one of items 54 to 56, the size of said buprenorphine-containing matrix layer providing the area of release ranging from more than 19 cm² to about 30 cm².
   58. Transdermal therapeutic system in accordance with item 57, the size of said buprenorphine-containing matrix layer providing the area of release ranging from about 20 cm² to about 26 cm².
   59. Transdermal therapeutic system in accordance with item 57, the size of said buprenorphine-containing matrix layer providing the area of release ranging from about 20 cm² to about 24 cm².
   60. Transdermal therapeutic system in accordance with any one of items 21, 26, 31, or 54 to 59, said transdermal therapeutic system providing a mean AUCt of more than 28,000 pg·hr/ml over about 168 hours of administration after a single-dose administration to a subject population.
   61. Transdermal therapeutic system in accordance with item 60, said transdermal therapeutic system providing a mean AUCt of more than 32,000 pg·hr/ml over about 168 hours of administration after a single-dose administration to a subject population.
   62. Transdermal therapeutic system in accordance with item 60, said transdermal therapeutic system providing a mean AUCt of from more than 32,000 pg·hr/ml to about 64,000 pg·hr/ml over about 168 hours of administration after a single-dose administration to a subject population.
   63. Transdermal therapeutic system in accordance with any one of items 21, 26, 31, or 54 to 62, said transdermal therapeutic system providing a mean release rate ranging from about 15 to about 25 μg/hr, and/or a nominal mean release rate of about 20 μg/hr over about 168 hours of administration.
   64. Transdermal therapeutic system in accordance with any one of items 1 to 12, the amount of said buprenorphine contained in the transdermal therapeutic system ranging from about 11.5 mg to about 24 mg buprenorphine base or an equimolar amount of a pharmaceutically acceptable salt thereof.
   65. Transdermal therapeutic system in accordance with item 64, the amount of said buprenorphine contained in the transdermal therapeutic system ranging from about 11.5 mg to about 21 mg buprenorphine base or an equimolar amount of a pharmaceutically acceptable salt thereof.
   66. Transdermal therapeutic system in accordance with item 64, the amount of said buprenorphine contained in the transdermal therapeutic system ranging from about 12.5 mg to about 18 mg buprenorphine base or an equimolar amount of a pharmaceutically acceptable salt thereof.
   67. Transdermal therapeutic system in accordance with any one of items 64 to 66, the size of said buprenorphine-containing matrix layer providing the area of release ranging from more than 28.5 cm² to about 45 cm².
   68. Transdermal therapeutic system in accordance with item 67, the size of said buprenorphine-containing matrix layer providing the area of release ranging from about 30 cm² to about 39 cm².
   69. Transdermal therapeutic system in accordance with item 67, the size of said buprenorphine-containing matrix layer providing the area of release ranging from about 30 cm² to about 36 cm².
   70. Transdermal therapeutic system in accordance with any one of items 22, 27, 32, or 64 to 69, said transdermal therapeutic system providing a mean AUCt of more than 42,000 pg·hr/ml over about 168 hours of administration after a single-dose administration to a subject population.
   71. Transdermal therapeutic system in accordance with item 70, said transdermal therapeutic system providing a mean AUCt of more than 48,000 pg·hr/ml over about 168 hours of administration after a single-dose administration to a subject population.
   72. Transdermal therapeutic system in accordance with item 70, said transdermal therapeutic system providing a mean AUCt of from more than 48,000 pg·hr/ml to about 96,000 pg·hr/ml over about 168 hours of administration after a single-dose administration to a subject population.
   73. Transdermal therapeutic system in accordance with any one of items 22, 27, 32, or 64 to 72, said transdermal therapeutic system providing a mean release rate ranging from about 26 to about 35 μg/hr, and/or a nominal mean release rate of about 30 μg/hr over about 168 hours of administration.
   74. Transdermal therapeutic system in accordance with any one of items 1 to 12, the amount of said buprenorphine contained in the transdermal therapeutic system ranging from about 15 mg to about 32 mg buprenorphine base or an equimolar amount of a pharmaceutically acceptable salt thereof.
   75. Transdermal therapeutic system in accordance with item 74, the amount of said buprenorphine contained in the transdermal therapeutic system ranging from about 15 mg to about 28 mg buprenorphine base or an equimolar amount of a pharmaceutically acceptable salt thereof.
   76. Transdermal therapeutic system in accordance with item 74, the amount of said buprenorphine contained in the transdermal therapeutic system ranging from about 18.5 mg to about 24 mg buprenorphine base or an equimolar amount of a pharmaceutically acceptable salt thereof.
   77. Transdermal therapeutic system in accordance with any one of items 74 to 76, the size of said buprenorphine-containing matrix layer providing the area of release ranging from more than 38 cm² to about 60 cm².
   78. Transdermal therapeutic system in accordance with item 77, the size of said buprenorphine-containing matrix layer providing the area of release ranging from about 40 cm² to about 52 cm².
   79. Transdermal therapeutic system in accordance with item 77, the size of said buprenorphine-containing matrix layer providing the area of release ranging from about 40 cm² to about 48 cm².
   80. Transdermal therapeutic system in accordance with any one of items 23, 28, 33, or 74 to 79, said transdermal therapeutic system providing a mean AUCt of more than 62,000 pg·hr/ml over about 168 hours of administration after a single-dose administration to a subject population.
   81. Transdermal therapeutic system in accordance with item 80, said transdermal therapeutic system providing a mean AUCt of more than 64,000 pg·hr/ml over about 168 hours of administration after a single-dose administration to a subject population.
   82. Transdermal therapeutic system in accordance with item 80, said transdermal therapeutic system providing a mean AUCt of from more than 64,000 pg·hr/ml to about 128,000 pg·hr/ml over about 168 hours of administration after a single-dose administration to a subject population.
   83. Transdermal therapeutic system in accordance with any one of items 23, 28, 33, or 74 to 82, said transdermal therapeutic system providing a mean release rate ranging from about 36 to about 45 μg/hr, and/or a nominal mean release rate of about 40 μg/hr over about 168 hours of administration.
   84. Transdermal therapeutic system in accordance with any one of items 1 to 83, said transdermal therapeutic system providing an arithmetic mean tmax from about 72 hr to about 132 hr after a single dose administration to a subject population.
   85. Transdermal therapeutic system in accordance with item 85, said transdermal therapeutic system providing an arithmetic mean tmax from about 78 hr to about 126 hr after a single dose administration to a subject population.
   86. Transdermal therapeutic system in accordance with item 85, said transdermal therapeutic system providing an arithmetic mean tmax from about 84 hr to about 120 hr after a single dose administration to a subject population.
   87. Transdermal therapeutic system in accordance with any one of items 1 to 87, said buprenorphine-containing self-adhesive layer structure containing more than 4% buprenorphine base or an equimolar amount of a pharmaceutically acceptable salt thereof based on the dry weight of the initial composition of the buprenorphine-containing matrix layer.
   88. Transdermal therapeutic system in accordance with item 87, said buprenorphine-containing self-adhesive layer structure containing more than 5% buprenorphine base or an equimolar amount of a pharmaceutically acceptable salt thereof based on the dry weight of the initial composition of the buprenorphine-containing matrix layer.
   89. Transdermal therapeutic system in accordance with item 87, said buprenorphine-containing self-adhesive layer structure containing more than 6% buprenorphine base or an equimolar amount of a pharmaceutically acceptable salt thereof based on the dry weight of the initial composition of the buprenorphine-containing matrix layer.
   90. Transdermal therapeutic system in accordance with item 87, said buprenorphine-containing self-adhesive layer structure containing more than 7% buprenorphine base or an equimolar amount of a pharmaceutically acceptable salt thereof based on the dry weight of the initial composition of the buprenorphine-containing matrix layer.
   91. Transdermal therapeutic system in accordance with any one of items 1 to 90, said buprenorphine-containing self-adhesive layer structure containing from about 5% to about 20% buprenorphine base or an equimolar amount of a pharmaceutically acceptable salt thereof based on the dry weight of the initial composition of the buprenorphine-containing matrix layer.
   92. Transdermal therapeutic system in accordance with item 91, said buprenorphine-containing self-adhesive layer structure containing from about 6% to about 20% buprenorphine base or an equimolar amount of a pharmaceutically acceptable salt thereof based on the dry weight of the initial composition of the buprenorphine-containing matrix layer.
   93. Transdermal therapeutic system in accordance with item 91, said buprenorphine-containing self-adhesive layer structure containing from about 7% to about 15% buprenorphine base or an equimolar amount of a pharmaceutically acceptable salt thereof based on the dry weight of the initial composition of the buprenorphine-containing matrix layer.
   94. Transdermal therapeutic system in accordance with any one of items 1 to 93, wherein said buprenorphine is present in the form of buprenorphine base.
   95. Transdermal therapeutic system in accordance with any one of items 1 to 94, wherein said carboxylic acid is levulinic acid.
   96. Transdermal therapeutic system in accordance with item 95, said buprenorphine-containing self-adhesive layer structure containing more than 4% levulinic acid based on the dry weight of the initial composition of the buprenorphine-containing matrix layer.
   97. Transdermal therapeutic system in accordance with item 96, said buprenorphine-containing self-adhesive layer structure containing more than 5% levulinic acid based on the dry weight of the initial composition of the buprenorphine-containing matrix layer.
   98. Transdermal therapeutic system in accordance with item 96, said buprenorphine-containing self-adhesive layer structure containing more than 6% levulinic acid based on the dry weight of the initial composition of the buprenorphine-containing matrix layer.
   99. Transdermal therapeutic system in accordance with item 96, said buprenorphine-containing self-adhesive layer structure containing more than 7% levulinic acid based on the dry weight of the initial composition of the buprenorphine-containing matrix layer.
   100. Transdermal therapeutic system in accordance with item 96, said buprenorphine-containing self-adhesive layer structure containing more than 8% levulinic acid based on the dry weight of the initial composition of the buprenorphine-containing matrix layer.
   101. Transdermal therapeutic system in accordance with item 96, said buprenorphine-containing self-adhesive layer structure containing 9% or more levulinic acid based on the dry weight of the initial composition of the buprenorphine-containing matrix layer.
   102. Transdermal therapeutic system in accordance with item 96, said buprenorphine-containing self-adhesive layer structure containing more than 9% levulinic acid based on the dry weight of the initial composition of the buprenorphine-containing matrix layer.
   103. Transdermal therapeutic system in accordance with item 95, said buprenorphine-containing self-adhesive layer structure containing from about 5% to about 20% levulinic acid based on the dry weight of the initial composition of the buprenorphine-containing matrix layer.
   104. Transdermal therapeutic system in accordance with item 103, said buprenorphine-containing self-adhesive layer structure containing from about 6% to about 20% levulinic acid based on the dry weight of the initial composition of the buprenorphine-containing matrix layer.
   105. Transdermal therapeutic system in accordance with item 103, said buprenorphine-containing self-adhesive layer structure containing from about 7% to about 15% levulinic acid based on the dry weight of the initial composition of the buprenorphine-containing matrix layer.
   106. Transdermal therapeutic system in accordance with item 103, said buprenorphine-containing self-adhesive layer structure containing from about 8% to about 15% levulinic acid based on the dry weight of the initial composition of the buprenorphine-containing matrix layer.
   107. Transdermal therapeutic system in accordance with item 103, said buprenorphine-containing self-adhesive layer structure containing from about 9% to about 15% levulinic acid based on the dry weight of the initial composition of the buprenorphine-containing matrix layer.
   108. Transdermal therapeutic system in accordance with any one of items 1 to 107, wherein said buprenorphine is present in the form of buprenorphine base and said carboxylic acid is levulinic acid.
   109. Transdermal therapeutic system in accordance with item 108, said buprenorphine-containing self-adhesive layer structure containing the same amounts of buprenorphine base and levulinic acid, based on the % amount of buprenorphine base.
   110. Transdermal therapeutic system in accordance with item 108, said buprenorphine-containing self-adhesive layer structure containing less % amounts of buprenorphine base than % amounts of levulinic acid, based on the % amount of buprenorphine base.
   111. Transdermal therapeutic system in accordance with item 108, said buprenorphine-containing self-adhesive layer structure containing from about 5% to about 20% buprenorphine base and from about 5% to about 20% levulinic acid based on the dry weight of the initial composition of the buprenorphine-containing matrix layer.
   112. Transdermal therapeutic system in accordance with item 108, said buprenorphine-containing self-adhesive layer structure containing from about 7% to about 15% buprenorphine base and from about 9% to about 15% levulinic acid based on the dry weight of the initial composition of the buprenorphine-containing matrix layer.
   113. Transdermal therapeutic system in accordance with any one of items 1 to 112, said buprenorphine-containing matrix layer being coated at a dry weight of less than 8 mg/cm².
   114. Transdermal therapeutic system in accordance with item 113, said buprenorphine-containing matrix layer being coated at a dry weight of less than 7 mg/cm².
   115. Transdermal therapeutic system in accordance with item 113, said buprenorphine-containing matrix layer being coated at a dry weight of up to 6 mg/cm².
   116. Transdermal therapeutic system in accordance with item 113, said buprenorphine-containing matrix layer being coated at a dry weight of less than 6 mg/cm².
   117. Transdermal therapeutic system in accordance with any one of items 1 to 113, said buprenorphine-containing matrix layer being coated at a dry weight ranging from about 3 mg/cm² to less than 8 mg/cm².
   118. Transdermal therapeutic system in accordance with item 117, said buprenorphine-containing matrix layer being coated at a dry weight ranging from about 4 mg/cm² to less than 8 mg/cm².
   119. Transdermal therapeutic system in accordance with item 117, said buprenorphine-containing matrix layer being coated at a dry weight ranging from about 5 mg/cm² to about 7 mg/cm².
   120. Transdermal therapeutic system in accordance with item 117, said buprenorphine-containing matrix layer being coated at a dry weight ranging from about 5.5 mg/cm² to about 6.5 mg/cm².
   121. Transdermal therapeutic system in accordance with any one of items 1 to 120, said buprenorphine-containing matrix layer being coated at a dry weight of about 6 mg/cm², and wherein said buprenorphine is present in the form of buprenorphine base and the buprenorphine-containing self-adhesive layer structure contains about 7.5% buprenorphine base based on the dry weight of the initial composition of the buprenorphine-containing matrix layer, and wherein the carboxylic acid is levulinic acid the buprenorphine-containing self-adhesive layer structure contains about 9% levulinic acid based on the dry weight of the initial composition of the buprenorphine-containing matrix layer.
   122. Transdermal therapeutic system in accordance with any one of items 1 to 120, said buprenorphine-containing matrix layer being coated at a dry weight of about 6 mg/cm², and wherein said buprenorphine is present in the form of buprenorphine base and the buprenorphine-containing self-adhesive layer structure contains about 7.5% buprenorphine base based on the dry weight of the initial composition of the buprenorphine-containing matrix layer, and wherein the carboxylic acid is levulinic acid the buprenorphine-containing self-adhesive layer structure contains about 10% levulinic acid based on the dry weight of the initial composition of the buprenorphine-containing matrix layer.
   123. Transdermal therapeutic system in accordance with any one of items 1 to 122, wherein said polymer base is a polymer-based pressure-sensitive adhesive.
   124. Transdermal therapeutic system in accordance with any one of items 1 to 123, wherein said polymer base is a polymer-based pressure-sensitive adhesive comprising polysiloxane or polyisobutylene.
   125. Transdermal therapeutic system in accordance with any one of items 1 to 124, wherein said polymer base is a polymer-based pressure-sensitive adhesive comprising polysiloxane.
   126. Transdermal therapeutic system in accordance with any one of items 1 to 125, wherein said polymer base is a polymer-based pressure-sensitive adhesive comprising polysiloxane being amine-resistant.
   127. Transdermal therapeutic system in accordance with any one of items 1 to 126, wherein said polymer base is a polymer-based pressure-sensitive adhesive comprising polysiloxane and the polysiloxane is amine-resistant being a product of the condensation reaction of silanol endblocked polydimethylsiloxane with a silica resin and the residual silanol functionality being capped with trimethylsiloxy groups.
   128. Transdermal therapeutic system in accordance with any one of items 1 to 127, wherein said polymer base is a polymer-based pressure-sensitive adhesive comprising polysiloxane and wherein for the production of said buprenorphine-containing matrix layer an adhesive composition of the pressure-sensitive adhesive comprising polysiloxane in heptane is used.
   129. Transdermal therapeutic system in accordance with any one of items 1 to 128, wherein said buprenorphine is present in the form of buprenorphine base, said carboxylic acid is levulinic acid and said polymer base is a polymer-based pressure-sensitive adhesive comprising polysiloxane.
   130. Transdermal therapeutic system in accordance with any one of items 1 to 129, wherein said skin contact layer comprises a polymer-based pressure-sensitive adhesive comprising polyacrylate.
   131. Transdermal therapeutic system in accordance with any one of items 1 to 130, wherein said skin contact layer comprises a polymer-based pressure-sensitive adhesive comprising polyacrylate prepared from 2-ethylhexyl acrylate, vinylacetate and 2-hydroxyethyl acrylate.
   132. Transdermal therapeutic system in accordance with any one of items 1 to 131, wherein said skin contact layer comprises a polymer-based pressure-sensitive adhesive comprising polyacrylate and wherein for the production of the skin contact layer an adhesive composition of the pressure-sensitive adhesive comprising polyacrylate in ethyl acetate is used.
   133. Transdermal therapeutic system in accordance with any one of items 1 to 132, wherein said buprenorphine is present in the form of buprenorphine base, said carboxylic acid is levulinic acid, said polymer base is a polymer-based pressure-sensitive adhesive comprising polysiloxane and said skin contact layer comprises a polymer-based pressure-sensitive adhesive comprising polyacrylate.
   134. Transdermal therapeutic system in accordance with any one of items 1 to 133, wherein said skin contact layer comprises a polymer-based pressure-sensitive adhesive comprising polysiloxane or polyisobutylene.
   135. Transdermal therapeutic system in accordance with any one of items 1 to 134, wherein said skin contact layer comprises a polymer-based pressure-sensitive adhesive comprising polysiloxane.
   136. Transdermal therapeutic system in accordance with any one of items 1 to 135, wherein said skin contact layer comprises a polymer-based pressure-sensitive adhesive comprising polysiloxane being amine-resistant.
   137. Transdermal therapeutic system in accordance with any one of items 1 to 136, wherein said skin contact layer comprises a polymer-based pressure-sensitive adhesive comprising polysiloxane and the polysiloxane is amine-resistant being a product of the condensation reaction of silanol endblocked polydimethylsiloxane with a silica resin and the residual silanol functionality being capped with trimethylsiloxy groups.
   138. Transdermal therapeutic system in accordance with any one of items 1 to 137, wherein said skin contact layer comprises a polymer-based pressure-sensitive adhesive comprising polysiloxane and wherein for the production of the skin contact layer an adhesive composition of the pressure-sensitive adhesive comprising polysiloxane in heptane is used.
   139. Transdermal therapeutic system in accordance with any one of items 1 to 138, said skin contact layer being coated at a dry weight of less than 6 mg/cm².
   140. Transdermal therapeutic system in accordance with item 139, said skin contact layer being coated at a dry weight of less than 5 mg/cm².
   141. Transdermal therapeutic system in accordance with item 139, said skin contact layer being coated at a dry weight of less than 4 mg/cm².
   142. Transdermal therapeutic system in accordance with any one of items 1 to 138, said skin contact layer being coated at a dry weight from about 1 mg/cm² to less than 6 mg/cm².
   143. Transdermal therapeutic system in accordance with item 142, said skin contact layer being coated at a dry weight from about 1 mg/cm² to about 5 mg/cm².
   144. Transdermal therapeutic system in accordance with item 142, said skin contact layer being coated at a dry weight from about 1 mg/cm² to about 4 mg/cm².
   145. Transdermal therapeutic system in accordance with item 142, said skin contact layer being coated at a dry weight from about 1 mg/cm² to about 3 mg/cm².
   146. Transdermal therapeutic system in accordance with item 142, said skin contact layer being coated at a dry weight from about 1.5 mg/cm² to about 2.5 mg/cm².
   147. Transdermal therapeutic system in accordance with any one of items 1 to 146, said buprenorphine-containing self-adhesive layer structure being attached to a larger active agent-free self-adhesive layer structure for enhancing the adhesive properties of the overall transdermal therapeutic system.
   148. Transdermal therapeutic system in accordance with item 147, said active agent-free self-adhesive layer structure comprising a buprenorphine-impermeable backing layer and an active agent-free pressure-sensitive adhesive layer of pressure-sensitive adhesive comprising polyacrylate.
   149. Transdermal therapeutic system in accordance with item 148, wherein said pressure-sensitive adhesive comprises polyacrylate prepared from 2-ethylhexyl acrylate, vinylacetate and 2-hydroxyethyl acrylate.
   150. Transdermal therapeutic system in accordance with item 148, wherein said pressure-sensitive adhesive comprises polyacrylate and wherein for the production of the active agent-free self-adhesive layer an adhesive composition of the pressure-sensitive adhesive comprising polyacrylate in ethyl acetate is used.
   151. Transdermal therapeutic system in accordance with item 147, said active agent-free self-adhesive layer structure comprising a buprenorphine-impermeable backing layer and an active agent-free pressure-sensitive adhesive layer of pressure-sensitive adhesive comprising polysiloxane.
   152. Transdermal therapeutic system in accordance with item 151, wherein said pressure-sensitive adhesive comprises polysiloxane being amine-resistant.
   153. Transdermal therapeutic system in accordance with item 151, wherein said pressure-sensitive adhesive comprises polysiloxane and the polysiloxane is amine-resistant being a product of the condensation reaction of silanol endblocked polydimethylsiloxane with a silica resin and the residual silanol functionality being capped with trimethylsiloxy groups.
   154. Transdermal therapeutic system in accordance with item 151, wherein said pressure-sensitive adhesive comprises polysiloxane and wherein for the production of active agent-free self-adhesive layer an adhesive composition of the pressure-sensitive adhesive comprising polysiloxane in heptane is used.
   155. Transdermal therapeutic system in accordance with any one of items 1 to 154, wherein said polymer-based pressure-sensitive adhesive comprises polysiloxane and is characterized by a solution viscosity at 25° C. and 60% solids content in heptane of more than about 150 mPa s.
   156. Transdermal therapeutic system in accordance with item 155, wherein said polymer-based pressure-sensitive adhesive comprises polysiloxane and is characterized by a solution viscosity at 25° C. and 60% solids content in heptane of from about 200 mPa s to about 700 mPa s.
   157. Transdermal therapeutic system in accordance with item 155, wherein said polymer-based pressure-sensitive adhesive comprises polysiloxane and is characterized by a solution viscosity at 25° C. and 60% solids content in heptane of from about 350 mPa s to about 600 mPa s.
   158. Transdermal therapeutic system in accordance with item 155, wherein said polymer-based pressure-sensitive adhesive comprises polysiloxane and is characterized by a solution viscosity at 25° C. and 60% solids content in heptane of from 480 mPa s to about 550 mPa s or alternatively from about 400 to less than 480 mPa s.
   159. Transdermal therapeutic system in accordance with item 155, wherein said polymer-based pressure-sensitive adhesive comprises polysiloxane and is characterized by a solution viscosity at 25° C. and 60% solids content in heptane of about 500 mPa s or alternatively of about 450 mPa s.
   160. Transdermal therapeutic system in accordance with any one of items 1 to 150, wherein said polymer-based pressure-sensitive adhesive comprises polyacrylate and is characterized by providing a 180° Peel at 20 minutes of at least about 20 N/25 mm, at 24 minutes of at least about 25 N/25 cm, at one week of at least about 30 N/25 mm and a Loop tack of at least 15 N/25 mm², or of at least 20 N/25 mm², or of at least 22 N/25 mm².
   161. Transdermal therapeutic system in accordance with any one of items 1 to 160, wherein buprenorphine is present in the form of buprenorphine base and said transdermal therapeutic system provides a mean cumulative skin permeation rate measured in a Franz diffusion cell with dermatomed human skin of more than 1.1 μg/cm²-hr over a 168 hours test.
   162. Transdermal therapeutic system in accordance with item 161, said transdermal therapeutic system providing a mean cumulative skin permeation rate measured in a Franz diffusion cell with dermatomed human skin of more than 1.2 μg/cm²-hr over a 168 hours test.
   163. Transdermal therapeutic system in accordance with item 161, said transdermal therapeutic system providing a mean cumulative skin permeation rate measured in a Franz diffusion cell with dermatomed human skin of more than 1.3 μg/cm²-hr over a 168 hours test.
   164. Transdermal therapeutic system in accordance with item 161, said transdermal therapeutic system providing a mean cumulative skin permeation rate measured in a Franz diffusion cell with dermatomed human skin of more than 1.4 μg/cm²-hr over a 168 hours test.
   165. Transdermal therapeutic system in accordance with item 161, said transdermal therapeutic system providing a mean cumulative skin permeation rate measured in a Franz diffusion cell with dermatomed human skin of 1.5 μg/cm²-hr or more over a 168 hours test.
   166. Transdermal therapeutic system in accordance with any one of items 1 to 160, wherein buprenorphine is present in the form of buprenorphine base and said transdermal therapeutic system provides a mean cumulative skin permeation rate measured in a Franz diffusion cell with dermatomed human skin from about 1.2 μg/cm²-hr to about 4 μg/cm over a 168 hours test.
   167. Transdermal therapeutic system in accordance with item 166, said transdermal therapeutic system providing a mean cumulative skin permeation rate measured in a Franz diffusion cell with dermatomed human skin from about 1.3 μg/cm²-hr to about 4 μg/cm²-hr over a 168 hours test.
   168. Transdermal therapeutic system in accordance with item 166, said transdermal therapeutic system providing a mean cumulative skin permeation rate measured in a Franz diffusion cell with dermatomed human skin from about 1.4 μg/cm²-hr to about 4 μg/cm²-hr over a 168 hours test.
   169. Transdermal therapeutic system in accordance with item 166, providing a mean cumulative skin permeation rate measured in a Franz diffusion cell with dermatomed human skin from about 1.5 μg/cm²-hr to about 2 μg/cm²-hr over a 168 hours test.
   170. Transdermal therapeutic system in accordance with any one of items 1 to 169, wherein buprenorphine is present in the form of buprenorphine base and said transdermal therapeutic system provides a cumulative release of buprenorphine base as measured in a Franz diffusion cell with dermatomed human skin of more than 185 μg/cm² over a time period of 168 hours.
   171. Transdermal therapeutic system in accordance with item 170, said transdermal therapeutic system providing a cumulative release of buprenorphine base as measured in a Franz diffusion cell with dermatomed human skin of more than 200 μg/cm² over a time period of 168 hours.
   172. Transdermal therapeutic system in accordance with item 170, said transdermal therapeutic system providing a cumulative release of buprenorphine base as measured in a Franz diffusion cell with dermatomed human skin of more than 220 μg/cm² over a time period of 168 hours.
   173. Transdermal therapeutic system in accordance with item 170, said transdermal therapeutic system providing a cumulative release of buprenorphine base as measured in a Franz diffusion cell with dermatomed human skin of more than 235 μg/cm² over a time period of 168 hours.
   174. Transdermal therapeutic system in accordance with item 170, said transdermal therapeutic system providing a cumulative release of buprenorphine base as measured in a Franz diffusion cell with dermatomed human skin of more than 250 μg/cm² over a time period of 168 hours.
   175. Transdermal therapeutic system in accordance with any one of items 1 to 169, wherein buprenorphine is present in the form of buprenorphine base and said transdermal therapeutic system provides a cumulative release of buprenorphine base as measured in a Franz diffusion cell with dermatomed human skin from about 200 μg/cm² to about 400 μg/cm² and more over a time period of 168 hours.
   176. Transdermal therapeutic system in accordance with item 175, said transdermal therapeutic system providing a cumulative release of buprenorphine base as measured in a Franz diffusion cell with dermatomed human skin from about 220 μg/cm² to about 350 μg/cm² over a time period of 168 hours.
   177. Transdermal therapeutic system in accordance with item 175, said transdermal therapeutic system providing a cumulative release of buprenorphine base as measured in a Franz diffusion cell with dermatomed human skin from about 235 μg/cm² to about 300 μg/cm² over a time period of 168 hours.
   178. Transdermal therapeutic system in accordance with item 175, said transdermal therapeutic system providing a cumulative release of buprenorphine base as measured in a Franz diffusion cell with dermatomed human skin from about 250 μg/cm² to about 300 μg/cm² over a time period of 168 hours.
   179. Transdermal therapeutic system in accordance with any one of items 1 to 178, wherein buprenorphine is present in the form of buprenorphine base and said transdermal therapeutic system provides a non-cumulative release of buprenorphine base as measured in a Franz diffusion cell with dermatomed human skin of
   1 μg/cm² to 10 μg/cm² in the first 8 hours,
   10 μg/cm² to 60 μg/cm² from hour 8 to hour 24,
   10 μg/cm² to 60 μg/cm² from hour 24 to hour 32,
   30 μg/cm² to 100 μg/cm² from hour 32 to hour 48,
   40 μg/cm² to 120 μg/cm² from hour 48 to hour 72,
   50 μg/cm² to 150 μg/cm² from hour 72 to hour 144, and
   10 μg/cm² to 50 μg/cm² from hour 144 to hour 168.
   180. Transdermal therapeutic system in accordance with item 179, said transdermal therapeutic system providing a non-cumulative release of buprenorphine base as measured in a Franz diffusion cell with dermatomed human skin of
   1 μg/cm² to 6 μg/cm in the first 8 hours,
   15 μg/cm² to 50 μg/cm² from hour 8 to hour 24,
   15 μg/cm² to 50 μg/cm² from hour 24 to hour 32,
   40 μg/cm² to 80 μg/cm² from hour 32 to hour 48,
   50 μg/cm² to 100 μg/cm² from hour 48 to hour 72,
   60 μg/cm² to 120 μg/cm² from hour 72 to hour 144, and
   15 μg/cm² to 40 μg/cm² from hour 144 to hour 168.
   181. Transdermal therapeutic system in accordance with item 179, said transdermal therapeutic system providing a non-cumulative release of buprenorphine base as measured in a Franz diffusion cell with dermatomed human skin of
   1 μg/cm² to 4 μg/cm in the first 8 hours,
   20 μg/cm² to 40 μg/cm² from hour 8 to hour 24,
   20 μg/cm² to 40 μg/cm² from hour 24 to hour 32,
   40 μg/cm² to 60 μg/cm² from hour 32 to hour 48,
   50 μg/cm² to 80 μg/cm² from hour 48 to hour 72,
   60 μg/cm² to 100 μg/cm² from hour 72 to hour 144, and
   15 μg/cm² to 30 μg/cm² from hour 144 to hour 168.
   182. Transdermal therapeutic system, comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
   and optionally wherein the buprenorphine-containing self-adhesive layer structure contains said buprenorphine in an amount of less than 0.8 mg/cm² buprenorphine base or an equimolar amount of a pharmaceutically acceptable salt thereof,
   and wherein said buprenorphine-containing self-adhesive layer structure contains more than 9% levulinic acid based on the dry weight of the initial composition of the buprenorphine-containing matrix layer.
   183. Transdermal therapeutic system, comprising a buprenorphine base-containing self-adhesive layer structure comprising
A) a buprenorphine base-impermeable backing layer, and
B) a buprenorphine base-containing matrix layer on said buprenorphine base-impermeable backing layer, the matrix layer comprising
   a) a polymer-based pressure-sensitive adhesive comprising polysiloxane,
   b) buprenorphine base, and
   c) levulinic acid, in an amount sufficient so that said buprenorphine base is solubilized therein to form a mixture, and the levulinic acid buprenorphine base mixture forms dispersed deposits in the said pressure-sensitive adhesive, and
C) a skin contact layer on said buprenorphine base-containing matrix layer comprising a polymer-based pressure-sensitive adhesive comprising polyacrylate,
   wherein the buprenorphine base-containing self-adhesive layer structure contains said buprenorphine base in an amount of less than 0.8 mg/cm².
   184. Transdermal therapeutic system comprising buprenorphine for the transdermal administration of buprenorphine selected from:
   a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a mean AUCt of more than 7,000 pg·hr/ml over about 168 hours of administration after a single-dose administration to a subject population; and
   a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a mean AUCt of more than 14,000 pg·hr/ml over about 168 hours of administration after a single-dose administration to a subject population; and
   a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a mean AUCt of more than 28,000 pg·hr/ml over about 168 hours of administration after a single-dose administration to a subject population; and
   a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a mean AUCt of more than 42,000 pg·hr/ml over about 168 hours of administration after a single-dose administration to a subject population; and
   a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a mean AUCt of more than 62,000 pg·hr/ml over about 168 hours of administration after a single-dose administration to a subject population.
   185. Transdermal therapeutic system in accordance with item 184, wherein the first transdermal therapeutic system contains an amount of said buprenorphine ranging from about 1 mg to about 3.5 mg buprenorphine base or an equimolar amount of a pharmaceutically acceptable salt thereof and provides a size of the area of release ranging from about 5 cm² to about 7 cm²; and
   the second transdermal therapeutic system contains an amount of said buprenorphine ranging from about 3.5 mg to about 7 mg buprenorphine base or an equimolar amount of a pharmaceutically acceptable salt thereof and provides a size of the area of release ranging from about 10 cm² to about 13 cm²; and
   the third transdermal therapeutic system contains an amount of said buprenorphine ranging from about 6.5 mg to about 14 mg buprenorphine base or an equimolar amount of a pharmaceutically acceptable salt thereof and provides a size of the area of release ranging from about 20 cm² to about 26 cm²; and
   the fourth transdermal therapeutic system contains an amount of said buprenorphine ranging from about 11.5 mg to about 21 mg buprenorphine base or an equimolar amount of a pharmaceutically acceptable salt thereof and provides a size of the area of release ranging about 30 cm² to about 39 cm²; and
   the fifth transdermal therapeutic system contains an amount of said buprenorphine ranging from about 15 mg to about 28 mg buprenorphine base or an equimolar amount of a pharmaceutically acceptable salt thereof and provides a size of the area of release ranging from about 40 cm² to about 52 cm².
   186. Transdermal therapeutic system in accordance with item 184, wherein the first transdermal therapeutic system contains an amount of said buprenorphine ranging from about 1 mg to about 3 mg buprenorphine base or an equimolar amount of a pharmaceutically acceptable salt thereof and provides a size of the area of release ranging from about 5 cm² to about 6 cm²; and
   the second transdermal therapeutic system contains an amount of said buprenorphine ranging from about 3.5 mg to about 6 mg buprenorphine base or an equimolar amount of a pharmaceutically acceptable salt thereof and provides a size of the area of release ranging from about 10 cm² to about 12 cm²; and
   the third transdermal therapeutic system contains an amount of said buprenorphine ranging from about 6.5 mg to about 12 mg buprenorphine base or an equimolar amount of a pharmaceutically acceptable salt thereof and provides a size of the area of release ranging from about 20 cm² to about 24 cm²; and
   the fourth transdermal therapeutic system contains an amount of said buprenorphine ranging from about 12.5 mg to about 18 mg buprenorphine base or an equimolar amount of a pharmaceutically acceptable salt thereof and provides a size of the area of release ranging about 30 cm² to about 36 cm²; and
   the fifth transdermal therapeutic system contains an amount of said buprenorphine ranging from about 18.5 mg to about 24 mg buprenorphine base or an equimolar amount of a pharmaceutically acceptable salt thereof and provides a size of the area of release ranging from about 40 cm² to about 48 cm².
   187. Transdermal therapeutic system in accordance with any one of items 184 to 186, wherein the first transdermal therapeutic system provides a mean AUCt of more than 8,000 pg·hr/ml over about 168 hours of administration after a single-dose administration to a subject population; and
   the second transdermal therapeutic system provides a mean AUCt of more than 16,000 pg·hr/ml over about 168 hours of administration after a single-dose administration to a subject population; and
   the third transdermal therapeutic system provides a mean AUCt of more than 32,000 pg·hr/ml over about 168 hours of administration after a single-dose administration to a subject population; and
   the fourth transdermal therapeutic system provides a mean AUCt of more than 48,000 pg·hr/ml over about 168 hours of administration after a single-dose administration to a subject population; and
   the fifth transdermal therapeutic system provides a mean AUCt of more than 64,000 pg·hr/ml over about 168 hours of administration after a single-dose administration to a subject population.
   188. A set of transdermal therapeutic systems including at least two transdermal therapeutic systems selected from the first, second, third, fourth and fifth transdermal therapeutic system in accordance with any one of items 184 to 187.
   189. Transdermal therapeutic system in accordance with any one of items 1 to 188 for use in a method of treating pain in a patient by applying said transdermal therapeutic system for 7 days on the skin of a patient.
   190. Transdermal therapeutic system in accordance with item 189 by applying said transdermal therapeutic system for 168 hours on the skin of a patient.
   191. Method of treating pain in a patient by applying a transdermal therapeutic system in accordance with any one of items 1 to 188 for 7 days on the skin of a patient.
   192. Method of treating pain in a patient by applying a transdermal therapeutic system in accordance with any one of items 1 to 188 for 168 hours on the skin of a patient.
   193. Method of manufacture of a transdermal therapeutic system for the transdermal administration of buprenorphine in accordance with any one of items 1 to 190, comprising the steps of
1. providing a buprenorphine-containing composition comprising
   a) a polymer
   b) buprenorphine base or a pharmaceutically acceptable salt thereof
   c) a carboxylic acid, and
   d) solvent;
2. coating said buprenorphine-containing composition on a film in an amount to provide the desired coating dry weight,
3. drying said coated buprenorphine-containing composition to provide a buprenorphine-containing matrix layer with the desired coating dry weight,
4. laminating said buprenorphine-containing matrix layer to a backing layer,
5. providing an adhesive composition comprising a polymer-based pressure-sensitive adhesive,
6. coating said adhesive composition on a film in an amount to provide the desired coating dry weight,
7. drying said coated adhesive composition to provide a skin contact layer with the desired coating dry weight,
8. removing said film from the buprenorphine-containing matrix layer of step 4 and laminating said buprenorphine-containing matrix layer to said skin contact layer of step 7 to provide the buprenorphine-containing self-adhesive layer structure,
9. punching the individual systems from the buprenorphine-containing self-adhesive layer structure with the desired area of release, and
10. optionally adhering to the individual systems an active agent-free self-adhesive layer structure comprising also a backing layer and an active agent-free pressure-sensitive adhesive layer larger than the individual systems of the buprenorphine-containing self-adhesive layer structure.
   194. A set of two to five different transdermal therapeutic systems for the transdermal administration of buprenorphine selected from five different transdermal therapeutic systems, a first, a second, a third, a forth and a fifth transdermal therapeutic system, each of the five different transdermal therapeutic systems comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
   a) a polymer base,
   b) buprenorphine, and
   c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
   and optionally wherein the buprenorphine-containing self-adhesive layer structure contains said buprenorphine in an amount of less than 0.8 mg/cm² buprenorphine base or an equimolar amount of a pharmaceutically acceptable salt thereof, and wherein
   the first transdermal therapeutic system contains an amount of said buprenorphine ranging from about 1 mg to about 4 mg buprenorphine base or an equimolar amount of a pharmaceutically acceptable salt thereof and provides a size of said buprenorphine-containing matrix layer providing the area of release ranging from more than 4.8 cm² to about 8 cm²;
   the second transdermal therapeutic system contains an amount of said buprenorphine ranging from about 3.5 mg to about 8 mg buprenorphine base or an equimolar amount of a pharmaceutically acceptable salt thereof and provides a size of said buprenorphine-containing matrix layer providing the area of release ranging from more than 9.5 cm² to about 15 cm²; and
   the third transdermal therapeutic system contains an amount of said buprenorphine ranging from about 6.5 mg to about 16 mg buprenorphine base or an equimolar amount of a pharmaceutically acceptable salt thereof and provides a size of said buprenorphine-containing matrix layer providing the area of release ranging from more than 19 cm² to about 30 cm²; and
   the fourth transdermal therapeutic system contains an amount of said buprenorphine ranging from about 11.5 mg to about 24 mg buprenorphine base or an equimolar amount of a pharmaceutically acceptable salt thereof and provides a size of said buprenorphine-containing matrix layer providing the area of release ranging from more than 28.5 cm² to about 45 cm²; and
   the fifth transdermal therapeutic system contains an amount of said buprenorphine ranging from about 15 mg to about 32 mg buprenorphine base or an equimolar amount of a pharmaceutically acceptable salt thereof and provides a size of said buprenorphine-containing matrix layer providing the area of release ranging from more than 38 cm² to about 60 cm², wherein the five different transdermal therapeutic systems have increasing areas of release and amounts of buprenorphine from the first to the fifth transdermal therapeutic system.
   195. A set in accordance with item 194, wherein
   the first transdermal therapeutic system contains an amount of said buprenorphine ranging from about 1 mg to about 3.5 mg buprenorphine base or an equimolar amount of a pharmaceutically acceptable salt thereof and provides a size of said buprenorphine-containing matrix layer providing the area of release ranging from about 5 cm² to about 7 cm²;
   the second transdermal therapeutic system contains an amount of said buprenorphine ranging from about 3.5 mg to about 7 mg buprenorphine base or an equimolar amount of a pharmaceutically acceptable salt thereof and provides a size of said buprenorphine-containing matrix layer providing the area of release ranging from about 10 cm² to about 13 cm²; and
   the third transdermal therapeutic system contains an amount of said buprenorphine ranging from about 6.5 mg to about 14 mg buprenorphine base or an equimolar amount of a pharmaceutically acceptable salt thereof and provides a size of said buprenorphine-containing matrix layer providing the area of release ranging from about 20 cm² to about 26 cm²; and
   the fourth transdermal therapeutic system contains an amount of said buprenorphine ranging from about 11.5 mg to about 21 mg buprenorphine base or an equimolar amount of a pharmaceutically acceptable salt thereof and provides a size of said buprenorphine-containing matrix layer providing the area of release ranging from about 30 cm² to about 39 cm²; and
   the fifth transdermal therapeutic system contains an amount of said buprenorphine ranging from about 15 mg to about 28 mg buprenorphine base or an equimolar amount of a pharmaceutically acceptable salt thereof and provides a size of said buprenorphine-containing matrix layer providing the area of release ranging from about 40 cm² to about 52 cm².
   196. A set in accordance with item 194, wherein
   the first transdermal therapeutic system contains an amount of said buprenorphine ranging from about 1 mg to about 3 mg buprenorphine base or an equimolar amount of a pharmaceutically acceptable salt thereof and provides a size of said buprenorphine-containing matrix layer providing the area of release ranging from about 5 cm² to about 6 cm²;
   the second transdermal therapeutic system containing an amount of said buprenorphine ranging from about 3.5 mg to about 6 mg buprenorphine base or an equimolar amount of a pharmaceutically acceptable salt thereof and provides a size of said buprenorphine-containing matrix layer providing the area of release ranging from about 10 cm² to about 12 cm²; and
   the third transdermal therapeutic system contains an amount of said buprenorphine ranging from about 6.5 mg to about 12 mg buprenorphine base or an equimolar amount of a pharmaceutically acceptable salt thereof and provides a size of said buprenorphine-containing matrix layer providing the area of release ranging from about 20 cm² to about 24 cm²; and
   the fourth transdermal therapeutic system contains an amount of said buprenorphine ranging from about 12.5 mg to about 18 mg buprenorphine base or an equimolar amount of a pharmaceutically acceptable salt thereof and provides a size of said buprenorphine-containing matrix layer providing the area of release ranging from about 30 cm² to about 36 cm²; and
   the fifth transdermal therapeutic system contains an amount of said buprenorphine ranging from about 18.5 mg to about 24 mg buprenorphine base or an equimolar amount of a pharmaceutically acceptable salt thereof and provides a size of said buprenorphine-containing matrix layer providing the area of release ranging from about 40 cm² to about 48 cm².
   197. Transdermal therapeutic system selected from a set in accordance with any one of items 194 to 196 for use in a method of treating pain in a patient by applying said selected transdermal therapeutic system for 7 days on the skin of a patient.
   198. Method of treating pain in a patient by applying a transdermal therapeutic system selected from a set in accordance with any one of items 194 to 196 for 7 days on the skin of a patient.

1. A method of treating pain in a patient by applying a transdermal therapeutic system for the transdermal administration of buprenorphine for 7 days on the skin of a patient, said transdermal therapeutic system comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
a) a polymer base,
b) buprenorphine, and
c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
and optionally wherein the buprenorphine-containing self-adhesive layer structure contains said buprenorphine in an amount of less than 0.8 mg/cm² buprenorphine base or an equimolar amount of a pharmaceutically acceptable salt thereof.

2. The method of treating pain in accordance with claim 1, the amount of said buprenorphine contained in the transdermal therapeutic system ranging from about 1 mg to about 4 mg buprenorphine base or an equimolar amount of a pharmaceutically acceptable salt thereof, or
about 3.5 mg to about 8 mg buprenorphine base or an equimolar amount of a pharmaceutically acceptable salt thereof, or
about 6.5 mg to about 16 mg buprenorphine base or an equimolar amount of a pharmaceutically acceptable salt thereof, or
about 11.5 mg to about 24 mg buprenorphine base or an equimolar amount of a pharmaceutically acceptable salt thereof, or
about 15 mg to about 32 mg buprenorphine base or an equimolar amount of a pharmaceutically acceptable salt thereof.

3. The method of treating pain in accordance with claim 1 or 2, the size of said buprenorphine-containing matrix layer providing the area of release ranging from
more than 4.8 cm² to about 8 cm², or
more than 9.5 cm² to about 15 cm², or
more than 19 cm² to about 30 cm², or
more than 28.5 cm² to about 45 cm², or
more than 38 cm² to about 60 cm².

4. The method of treating pain in accordance with claim 1, the amount of said buprenorphine contained in the transdermal therapeutic system ranging from about 1 mg to about 4 mg buprenorphine base or an equimolar amount of a pharmaceutically acceptable salt thereof and the size of said buprenorphine-containing matrix layer providing the area of release ranging from more than 4.8 cm² to about 8 cm².

5. The method of treating pain in accordance with claim 4, said transdermal therapeutic system providing a mean AUCt of more than 7,000 pg·hr/ml over about 168 hours of administration after a single dose administration to a subject population.

6. The method of treating pain in accordance with claim 4 or 5, said transdermal therapeutic system providing a nominal mean release rate of about 5 μg/hr over about 168 hours of administration.

7. The method of treating pain in accordance with claim 1, the amount of said buprenorphine contained in the transdermal therapeutic system ranging from about 3.5 mg to about 8 mg buprenorphine base or an equimolar amount of a pharmaceutically acceptable salt thereof and the size of said buprenorphine-containing matrix layer providing the area of release ranging from more than 9.5 cm² to about 15 cm².

8. The method of treating pain in accordance with claim 7, said transdermal therapeutic system providing a mean AUCt of more than 14,000 pg·hr/ml over about 168 hours of administration after a single dose administration to a subject population.

9. The method of treating pain in accordance with claim 7 or 8, said transdermal therapeutic system providing a nominal mean release rate of about 10 μg/hr over about 168 hours of administration.

10. The method of treating pain in accordance with claim 1, the amount of said buprenorphine contained in the transdermal therapeutic system ranging from about 6.5 mg to about 16 mg buprenorphine base or an equimolar amount of a pharmaceutically acceptable salt thereof and the size of said buprenorphine-containing matrix layer providing the area of release ranging from more than 19 cm² to about 30 cm².

11. The method of treating pain in accordance with claim 10, said transdermal therapeutic system providing a mean AUCt of more than 28,000 pg·hr/ml over about 168 hours of administration after a single dose administration to a subject population.

12. The method of treating pain in accordance with claim 10 or 11, said transdermal therapeutic system providing a nominal mean release rate of about 20 μg/hr over about 168 hours of administration.

13. The method of treating pain in accordance with claim 1, the amount of said buprenorphine contained in the transdermal therapeutic system ranging from about 11.5 mg to about 24 mg buprenorphine base or an equimolar amount of a pharmaceutically acceptable salt thereof and the size of said buprenorphine-containing matrix layer providing the area of release ranging from more than 28.5 cm² to about 45 cm².

14. The method of treating pain in accordance with claim 13, said transdermal therapeutic system providing a mean AUCt of more than 42,000 pg·hr/ml over about 168 hours of administration after a single dose administration to a subject population.

15. The method of treating pain in accordance with claim 13 or 14, said transdermal therapeutic system providing a nominal mean release rate of about 30 μg/hr over about 168 hours of administration.

16. The method of treating pain in accordance with claim 1, the amount of said buprenorphine contained in the transdermal therapeutic system ranging from about 15 mg to about 32 mg buprenorphine base or an equimolar amount of a pharmaceutically acceptable salt thereof and the size of said buprenorphine-containing matrix layer providing the area of release ranging from more than 38 cm² to about 60 cm².

17. The method of treating pain in accordance with claim 16, said transdermal therapeutic system providing a mean AUCt of more than 62,000 pg·hr/ml over about 168 hours of administration after a single dose administration to a subject population.

18. The method of treating pain in accordance with claim 16 or 17, said transdermal therapeutic system providing a nominal mean release rate of about 40 μg/hr over about 168 hours of administration.

19. The method of treating pain in accordance with any one of claims 1 to 18, said transdermal therapeutic system providing an arithmetic mean tmax from about 72 hr to about 132 hr after a single dose administration to a subject population.

20. The method of treating pain in accordance with any one of claims 1 to 19, wherein said buprenorphine is present in the form of buprenorphine base.

21. The method of treating pain in accordance with any one of claims 1 to 20, wherein said carboxylic acid is levulinic acid.

22. The method of treating pain in accordance with any one of claims 1 to 21, wherein said buprenorphine is present in the form of buprenorphine base and said carboxylic acid is levulinic acid.

23. The method of treating pain in accordance with claim 22, said buprenorphine-containing self-adhesive layer structure containing the same % amounts of buprenorphine base and levulinic acid, based on the % amount of buprenorphine base.

24. The method of treating pain in accordance with claim 22, said buprenorphine-containing self-adhesive layer structure containing less % amounts of buprenorphine base than % amounts of levulinic acid, based on the % amount of buprenorphine base.

25. The method of treating pain in accordance with any one of claims 1 to 24, said buprenorphine-containing matrix layer being coated at a dry weight of less than 8 mg/cm².

26. The method of treating pain in accordance with any one of claims 1 to 25, wherein said polymer base is a polymer-based pressure-sensitive adhesive.

27. The method of treating pain in accordance with any one of claims 1 to 26, wherein said polymer base is a polymer-based pressure-sensitive adhesive comprising polysiloxane or polyisobutylene.

28. The method of treating pain in accordance with any one of claims 1 to 27, wherein said buprenorphine is present in the form of buprenorphine base, said carboxylic acid is levulinic acid and said polymer base is a polymer-based pressure-sensitive adhesive comprising polysiloxane.

29. The method of treating pain in accordance with any one of claims 1 to 28, wherein said skin contact layer comprises a polymer-based pressure-sensitive adhesive comprising polyacrylate.

30. The method of treating pain in accordance with any one of claims 1 to 29, wherein said buprenorphine is present in the form of buprenorphine base, said carboxylic acid is levulinic acid, said polymer base is a polymer-based pressure-sensitive adhesive comprising polysiloxane and said skin contact layer comprises a polymer-based pressure-sensitive adhesive comprising polyacrylate.

31. The method of treating pain in accordance with any one of claims 1 to 30, said buprenorphine-containing self-adhesive layer structure being attached to a larger active agent-free self-adhesive layer structure for enhancing the adhesive properties of the overall transdermal therapeutic system.

32. The method of treating pain in accordance with claim 31, said active agent-free self-adhesive layer structure comprising a buprenorphine-impermeable backing layer and an active agent-free pressure-sensitive adhesive layer of pressure-sensitive adhesive comprising polyacrylate.

33. The method of treating pain in accordance with any one of claims 1 to 32, wherein buprenorphine is present in the form of buprenorphine base and said transdermal therapeutic system provides a mean cumulative skin permeation rate measured in a Franz diffusion cell with dermatomed human skin of more than 1.1 μg/cm²-hr over a 168 hours test.

34. The method of treating pain in accordance with any one of claims 1 to 33, wherein buprenorphine is present in the form of buprenorphine base and said transdermal therapeutic system provides a cumulative release of buprenorphine base as measured in a Franz diffusion cell with dermatomed human skin of more than 185 μg/cm² over a time period of 168 hours.

35. The method of treating pain in accordance with any one of claims 1 to 34, wherein buprenorphine is present in the form of buprenorphine base and said transdermal therapeutic system provides a non-cumulative release of buprenorphine base as measured in a Franz diffusion cell with dermatomed human skin of
1 μg/cm² to 10 μg/cm² in the first 8 hours,
10 μg/cm² to 60 μg/cm² from hour 8 to hour 24,
10 μg/cm² to 60 μg/cm² from hour 24 to hour 32,
30 μg/cm² to 100 μg/cm² from hour 32 to hour 48,
40 μg/cm² to 120 μg/cm² from hour 48 to hour 72,
50 μg/cm² to 150 μg/cm² from hour 72 to hour 144, and
10 μg/cm² to 50 μg/cm² from hour 144 to hour 168.

36. A method of treating pain in a patient by applying a transdermal therapeutic system for the transdermal administration of buprenorphine base for 7 days on the skin of a patient, said transdermal therapeutic system comprising a buprenorphine base-containing self-adhesive layer structure comprising
A) a buprenorphine base-impermeable backing layer, and
B) a buprenorphine base-containing matrix layer on said buprenorphine base-impermeable backing layer, the matrix layer comprising
a) a polymer-based pressure-sensitive adhesive comprising polysiloxane,
b) buprenorphine base, and
c) levulinic acid, in an amount sufficient so that said buprenorphine base is solubilized therein to form a mixture, and the levulinic acid buprenorphine base mixture forms dispersed deposits in the said pressure-sensitive adhesive, and
C) a skin contact layer on said buprenorphine base-containing matrix layer comprising a polymer-based pressure-sensitive adhesive comprising polyacrylate,
and optionally wherein the buprenorphine base-containing self-adhesive layer structure contains said buprenorphine base in an amount of less than 0.8 mg/cm².

37. Transdermal therapeutic system comprising buprenorphine for the transdermal administration of buprenorphine selected from:
a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a mean AUCt of more than 7,000 pg·hr/ml over about 168 hours of administration after a single-dose administration to a subject population; and
a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a mean AUCt of more than 14,000 pg·hr/ml over about 168 hours of administration after a single-dose administration to a subject population; and
a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a mean AUCt of more than 28,000 pg·hr/ml over about 168 hours of administration after a single-dose administration to a subject population; and
a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a mean AUCt of more than 42,000 pg·hr/ml over about 168 hours of administration after a single-dose administration to a subject population; and
a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a mean AUCt of more than 62,000 pg·hr/ml over about 168 hours of administration after a single-dose administration to a subject population.

38. Transdermal therapeutic system comprising buprenorphine for the transdermal administration of buprenorphine selected from:
a first transdermal therapeutic system containing an amount of said buprenorphine ranging from about 1 mg to about 4 mg buprenorphine base or an equimolar amount of a pharmaceutically acceptable salt thereof and providing a size of the area of release ranging from more than 4.8 cm² to about 8 cm² and providing a nominal mean release rate of about 5 μg/hr over about 168 hours of administration; and
a second transdermal therapeutic system containing an amount of said buprenorphine ranging from about 3.5 mg to about 8 mg buprenorphine base or an equimolar amount of a pharmaceutically acceptable salt thereof and providing a size of the area of release ranging from more than 9.5 cm² to about 15 cm² and providing a nominal mean release rate of about 10 μg/hr over about 168 hours of administration; and
a third transdermal therapeutic system containing an amount of said buprenorphine ranging from about 6.5 mg to about 16 mg buprenorphine base or an equimolar amount of a pharmaceutically acceptable salt thereof and providing a size of the area of release ranging from more than 19 cm² to about 30 cm² and providing a nominal mean release rate of about 20 μg/hr over about 168 hours of administration; and
a fourth transdermal therapeutic system containing an amount of said buprenorphine ranging from about 11.5 mg to about 24 mg buprenorphine base or an equimolar amount of a pharmaceutically acceptable salt thereof and providing a size of the area of release ranging from more than 28.5 cm² to about 45 cm² and providing a nominal mean release rate of about 30 μg/hr over about 168 hours of administration; and
a fifth transdermal therapeutic system containing an amount of said buprenorphine ranging from about 15 mg to about 32 mg buprenorphine base or an equimolar amount of a pharmaceutically acceptable salt thereof and providing a size of the area of release ranging from more than 38 cm² to about 60 cm² and providing a nominal mean release rate of about 40 μg/hr over about 168 hours of administration.

39. Transdermal therapeutic system in accordance with claim 37 or 38, containing buprenorphine in the area of release in an amount of less than 0.8 mg/cm² buprenorphine base or an equimolar amount of a pharmaceutically acceptable salt thereof.

40. A set of transdermal therapeutic systems including at least two transdermal therapeutic systems selected from the first, second, third, fourth and fifth transdermal therapeutic system in accordance with any one of claims 37 to 39.

41. A method of treating pain in a patient by
1. selecting for said patient the appropriate transdermal therapeutic system from the first, second, third, fourth and fifth transdermal therapeutic system in accordance with any one of claims 37 to 39; and
2. applying said selected transdermal therapeutic system on the skin of said patient for 7 days.

42. Transdermal therapeutic system for the transdermal administration of buprenorphine comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
a) a polymer base,
b) buprenorphine, and
c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
and optionally wherein the buprenorphine-containing self-adhesive layer structure contains said buprenorphine in an amount of less than 0.8 mg/cm² buprenorphine base or an equimolar amount of a pharmaceutically acceptable salt thereof,
for use in a method of treating pain in a patient by applying the transdermal therapeutic system for 7 days on the skin of a patient.

43. A method of manufacture of a transdermal therapeutic system for the transdermal administration of buprenorphine in accordance with claim 42, comprising the steps of
1. providing a buprenorphine-containing composition comprising
a) a polymer
b) buprenorphine base or a pharmaceutically acceptable salt thereof
c) a carboxylic acid, and
d) solvent;
2. coating said buprenorphine-containing composition on a film in an amount to provide the desired coating dry weight,
3. drying said coated buprenorphine-containing composition to provide a buprenorphine-containing matrix layer with the desired coating dry weight,
4. laminating said buprenorphine-containing matrix layer to a backing layer,
5. providing an adhesive composition comprising a polymer-based pressure-sensitive adhesive,
6. coating said adhesive composition on a film in an amount to provide the desired coating dry weight,
7. drying said coated adhesive composition to provide a skin contact layer with the desired coating dry weight,
8. removing said film from the buprenorphine-containing matrix layer of step 4 and laminating said buprenorphine-containing matrix layer to said skin contact layer of step 7 to provide the buprenorphine-containing self-adhesive layer structure,
9. punching the individual systems from the buprenorphine-containing self-adhesive layer structure with the desired area of release, and
10. optionally adhering to the individual systems an active agent-free self-adhesive layer structure comprising also a backing layer and an active agent-free pressure-sensitive adhesive layer larger than the individual systems of the buprenorphine-containing self-adhesive layer structure.

44. A set of two to five different transdermal therapeutic systems for the transdermal administration of buprenorphine selected from five different transdermal therapeutic systems, a first, a second, a third, a forth and a fifth transdermal therapeutic system, each of the five different transdermal therapeutic systems comprising a buprenorphine-containing self-adhesive layer structure comprising
A) a buprenorphine-impermeable backing layer, and
B) a buprenorphine-containing matrix layer on said buprenorphine-impermeable backing layer, the matrix layer comprising
a) a polymer base,
b) buprenorphine, and
c) a carboxylic acid selected from the group consisting of oleic acid, linoleic acid, linolenic acid, levulinic acid and mixtures thereof, in an amount sufficient so that said buprenorphine is solubilized therein to form a mixture, and the carboxylic acid buprenorphine mixture forms dispersed deposits in the polymer base, and
C) a skin contact layer on said buprenorphine-containing matrix layer comprising a polymer-based pressure-sensitive adhesive,
and optionally wherein the buprenorphine-containing self-adhesive layer structure contains said buprenorphine in an amount of less than 0.8 mg/cm² buprenorphine base or an equimolar amount of a pharmaceutically acceptable salt thereof,
and wherein
the first transdermal therapeutic system provides a size of said buprenorphine-containing matrix layer providing the area of release ranging from more than 4.8 cm² to about 8 cm²; and
the second transdermal therapeutic system provides a size of said buprenorphine-containing matrix layer providing the area of release ranging from more than 9.5 cm² to about 15 cm²; and
the third transdermal therapeutic system provides a size of said buprenorphine-containing matrix layer providing the area of release ranging from more than 19 cm² to about 30 cm²; and
the fourth transdermal therapeutic system provides a size of said buprenorphine-containing matrix layer providing the area of release ranging from more than 28.5 cm² to about 45 cm²; and
the fifth transdermal therapeutic system provides a size of said buprenorphine-containing matrix layer providing the area of release ranging from more than 38 cm² to about 60 cm²,
wherein the five different transdermal therapeutic systems have increasing areas of release from the first to the fifth transdermal therapeutic system.

45. A set in accordance with claim 44, wherein
the first transdermal therapeutic system contains an amount of said buprenorphine ranging from about 1 mg to about 4 mg buprenorphine base or an equimolar amount of a pharmaceutically acceptable salt thereof; and
the second transdermal therapeutic system contains an amount of said buprenorphine ranging from about 3.5 mg to about 8 mg buprenorphine base or an equimolar amount of a pharmaceutically acceptable salt thereof; and
the third transdermal therapeutic system contains an amount of said buprenorphine ranging from about 6.5 mg to about 16 mg buprenorphine base or an equimolar amount of a pharmaceutically acceptable salt thereof; and
the fourth transdermal therapeutic system contains an amount of said buprenorphine ranging from about 11.5 mg to about 24 mg buprenorphine base or an equimolar amount of a pharmaceutically acceptable salt thereof; and
the fifth transdermal therapeutic system contains an amount of said buprenorphine ranging from about 15 mg to about 32 mg buprenorphine base or an equimolar amount of a pharmaceutically acceptable salt thereof,
wherein the five different transdermal therapeutic systems have increasing amounts of buprenorphine from the first to the fifth transdermal therapeutic system.

46. A method of treating pain in a patient by
1. selecting for said patient the appropriate transdermal therapeutic system from the set in accordance with claim 44 or 45; and
2. applying said selected transdermal therapeutic system on the skin of said patient for 7 days.

47. Transdermal therapeutic system selected from a set in accordance with any one of claims 40, 44 and 45 for use in a method of treating pain in a patient by applying said selected transdermal therapeutic system for 7 days on the skin of the patient.
